SEQUENCE LISTING (121 sequences)

SEQ ID NO: 1-16 - skipped (16 entries)

SEQ ID NO: 17 - DNA, 23 bp, synthetic construct
  source 1..23: mol_type = other DNA
cctacactcgacatcggtagcta

SEQ ID NO: 18 - DNA, 23 bp, synthetic construct
  source 1..23: mol_type = other DNA
ccctggcggaacgttcgagtaat

SEQ ID NO: 19 - DNA, 23 bp, synthetic construct
  source 1..23: mol_type = other DNA
cctacacgatagtggtcgtgcgc

SEQ ID NO: 20 - DNA, 23 bp, synthetic construct
  source 1..23: mol_type = other DNA
cccgaactctctatcgaacgagt

SEQ ID NO: 21 - DNA, 23 bp, synthetic construct
  source 1..23: mol_type = other DNA
ccatggcacgatcgacacggagg

SEQ ID NO: 22 - DNA, 23 bp, synthetic construct
  source 1..23: mol_type = other DNA
ccaaagctcattcgcgactgagc

SEQ ID NO: 23 - DNA, 23 bp, synthetic construct
  source 1..23: mol_type = other DNA
ccgacgtctacggacgggagcta

SEQ ID NO: 24 - DNA, 23 bp, synthetic construct
  source 1..23: mol_type = other DNA
cccgagaggtagtagacgacgca

SEQ ID NO: 25 - skipped

SEQ ID NO: 26 - DNA, 23 bp, synthetic construct
  source 1..23: mol_type = other DNA
cccgtcgtcacgtatgcgtagca

SEQ ID NO: 27 - DNA, 23 bp, synthetic construct
  source 1..23: mol_type = other DNA
cccgtcgtcacgtatgcgtagca

SEQ ID NO: 28 - DNA, 23 bp, synthetic construct
  source 1..23: mol_type = other DNA
cccgagaggtagtagacgacgca

SEQ ID NO: 29 - DNA, 23 bp, synthetic construct
  source 1..23: mol_type = other DNA
ccgacgtctacggacgggagcta

SEQ ID NO: 30 - DNA, 23 bp, synthetic construct
  source 1..23: mol_type = other DNA
ccaaagctcattcgcgactgagc

SEQ ID NO: 31 - DNA, 23 bp, synthetic construct
  source 1..23: mol_type = other DNA
ccatggcacgatcgacacggagg

SEQ ID NO: 32 - DNA, 23 bp, synthetic construct
  source 1..23: mol_type = other DNA
cccgaactctctatcgaacgagt

SEQ ID NO: 33 - DNA, 23 bp, synthetic construct
  source 1..23: mol_type = other DNA
cctacacgatagtggtcgtgcgc

SEQ ID NO: 34 - DNA, 23 bp, synthetic construct
  source 1..23: mol_type = other DNA
ccctggcggaacgttcgagtaat

SEQ ID NO: 35 - DNA, 23 bp, synthetic construct
  source 1..23: mol_type = other DNA
cctacactcgacatcggtagcta

SEQ ID NO: 36 - DNA, 29 bp, synthetic construct
  source 1..29: mol_type = other DNA
ttttttttcagccaactccaatttttttt

SEQ ID NO: 37-44 - skipped (8 entries)

SEQ ID NO: 45 - DNA, 29 bp, synthetic construct
  source 1..29: mol_type = other DNA
ttttttttcagccaactccaatttttttt

SEQ ID NO: 46-53 - skipped (8 entries)

SEQ ID NO: 54 - DNA, 23 bp, synthetic construct
  source 1..23: mol_type = other DNA
tagctaccgatgtcgagtgtagg

SEQ ID NO: 55 - DNA, 23 bp, synthetic construct
  source 1..23: mol_type = other DNA
attactcgaacgttccgccaggg

SEQ ID NO: 56 - DNA, 23 bp, synthetic construct
  source 1..23: mol_type = other DNA
gcgcacgaccactatcgtgtagg

SEQ ID NO: 57 - DNA, 23 bp, synthetic construct
  source 1..23: mol_type = other DNA
actcgttcgatagagagttcggg

SEQ ID NO: 58 - DNA, 23 bp, synthetic construct
  source 1..23: mol_type = other DNA
cctccgtgtcgatcgtgccatgg

SEQ ID NO: 59 - DNA, 23 bp, synthetic construct
  source 1..23: mol_type = other DNA
gctcagtcgcgaatgagctttgg

SEQ ID NO: 60 - DNA, 23 bp, synthetic construct
  source 1..23: mol_type = other DNA
tagctcccgtccgtagacgtcgg

SEQ ID NO: 61 - DNA, 23 bp, synthetic construct
  source 1..23: mol_type = other DNA
tgcgtcgtctactacctctcggg

SEQ ID NO: 62 - DNA, 23 bp, synthetic construct
  source 1..23: mol_type = other DNA
tgctacgcatacgtgacgacggg

SEQ ID NO: 63 - DNA, 23 bp, synthetic construct
  source 1..23: mol_type = other DNA
tgctacgcatacgtgacgacggg

SEQ ID NO: 64 - DNA, 23 bp, synthetic construct
  source 1..23: mol_type = other DNA
tgcgtcgtctactacctctcggg

SEQ ID NO: 65 - DNA, 23 bp, synthetic construct
  source 1..23: mol_type = other DNA
tagctcccgtccgtagacgtcgg

SEQ ID NO: 66 - DNA, 23 bp, synthetic construct
  source 1..23: mol_type = other DNA
gctcagtcgcgaatgagctttgg

SEQ ID NO: 67 - DNA, 23 bp, synthetic construct
  source 1..23: mol_type = other DNA
cctccgtgtcgatcgtgccatgg

SEQ ID NO: 68 - DNA, 23 bp, synthetic construct
  source 1..23: mol_type = other DNA
actcgttcgatagagagttcggg

SEQ ID NO: 69 - DNA, 23 bp, synthetic construct
  source 1..23: mol_type = other DNA
gcgcacgaccactatcgtgtagg

SEQ ID NO: 70 - DNA, 23 bp, synthetic construct
  source 1..23: mol_type = other DNA
attactcgaacgttccgccaggg

SEQ ID NO: 71 - DNA, 23 bp, synthetic construct
  source 1..23: mol_type = other DNA
tagctaccgatgtcgagtgtagg

SEQ ID NO: 72 - DNA, 20 bp, synthetic construct
  source 1..20: mol_type = other DNA
tagctaccgatgtcgagtgt

SEQ ID NO: 73 - DNA, 20 bp, synthetic construct
  source 1..20: mol_type = other DNA
attactcgaacgttccgcca

SEQ ID NO: 74 - DNA, 20 bp, synthetic construct
  source 1..20: mol_type = other DNA
gcgcacgaccactatcgtgt

SEQ ID NO: 75 - DNA, 20 bp, synthetic construct
  source 1..20: mol_type = other DNA
actcgttcgatagagagttc

SEQ ID NO: 76 - DNA, 20 bp, synthetic construct
  source 1..20: mol_type = other DNA
cctccgtgtcgatcgtgcca

SEQ ID NO: 77 - DNA, 20 bp, synthetic construct
  source 1..20: mol_type = other DNA
gctcagtcgcgaatgagctt

SEQ ID NO: 78 - DNA, 20 bp, synthetic construct
  source 1..20: mol_type = other DNA
tagctcccgtccgtagacgt

SEQ ID NO: 79 - DNA, 20 bp, synthetic construct
  source 1..20: mol_type = other DNA
tgcgtcgtctactacctctc

SEQ ID NO: 80 - DNA, 20 bp, synthetic construct
  source 1..20: mol_type = other DNA
tgctacgcatacgtgacgac

SEQ ID NO: 81 - DNA, 20 bp, synthetic construct
  source 1..20: mol_type = other DNA
tgctacgcatacgtgacgac

SEQ ID NO: 82 - DNA, 20 bp, synthetic construct
  source 1..20: mol_type = other DNA
tgcgtcgtctactacctctc

SEQ ID NO: 83 - DNA, 20 bp, synthetic construct
  source 1..20: mol_type = other DNA
tagctcccgtccgtagacgt

SEQ ID NO: 84 - DNA, 20 bp, synthetic construct
  source 1..20: mol_type = other DNA
gctcagtcgcgaatgagctt

SEQ ID NO: 85 - DNA, 20 bp, synthetic construct
  source 1..20: mol_type = other DNA
cctccgtgtcgatcgtgcca

SEQ ID NO: 86 - DNA, 20 bp, synthetic construct
  source 1..20: mol_type = other DNA
actcgttcgatagagagttc

SEQ ID NO: 87 - DNA, 20 bp, synthetic construct
  source 1..20: mol_type = other DNA
gcgcacgaccactatcgtgt

SEQ ID NO: 88 - DNA, 20 bp, synthetic construct
  source 1..20: mol_type = other DNA
attactcgaacgttccgcca

SEQ ID NO: 89 - DNA, 20 bp, synthetic construct
  source 1..20: mol_type = other DNA
tagctaccgatgtcgagtgt

SEQ ID NO: 90 - DNA, 10 bp, synthetic construct
  source 1..10: mol_type = other DNA
tttttttttt

SEQ ID NO: 91 - DNA, 11 bp, synthetic construct
  source 1..11: mol_type = other DNA
ttttttttttt

SEQ ID NO: 92 - DNA, 12 bp, synthetic construct
  source 1..12: mol_type = other DNA
tttttttttttt

SEQ ID NO: 93 - DNA, 13 bp, synthetic construct
  source 1..13: mol_type = other DNA
ttttttttttttt

SEQ ID NO: 94 - DNA, 14 bp, synthetic construct
  source 1..14: mol_type = other DNA
tttttttttttttt

SEQ ID NO: 95 - DNA, 15 bp, synthetic construct
  source 1..15: mol_type = other DNA
ttttttttttttttt

SEQ ID NO: 96 - DNA, 20 bp, synthetic construct
  source 1..20: mol_type = other DNA
tttttttttttttttttttt

SEQ ID NO: 97 - DNA, 30 bp, synthetic construct
  source 1..30: mol_type = other DNA
tttttttttttttttttttttttttttttt

SEQ ID NO: 98 - DNA, 40 bp, synthetic construct
  source 1..40: mol_type = other DNA
tttttttttttttttttttttttttttttttttttttttt

SEQ ID NO: 99 - DNA, 50 bp, synthetic construct
  source 1..50: mol_type = other DNA
tttttttttttttttttttttttttttttttttttttttttttttttttt

SEQ ID NO: 100 - DNA, 60 bp, synthetic construct
  source 1..60: mol_type = other DNA
tttttttttttttttttttttttttttttttttttttttttttttttttttttttttttt

SEQ ID NO: 101 - DNA, 70 bp, synthetic construct
  source 1..70: mol_type = other DNA
tttttttttttttttttttttttttttttttttttttttttttttttttttttttttttttttttttttt

SEQ ID NO: 102 - DNA, 80 bp, synthetic construct
  source 1..80: mol_type = other DNA
tttttttttttttttttttttttttttttttttttttttttttttttttttttttttttttttttttttttttttttttt

SEQ ID NO: 103 - DNA, 90 bp, synthetic construct
  source 1..90: mol_type = other DNA
tttttttttttttttttttttttttttttttttttttttttttttttttttttttttttttttttttttttttttttttttttttttttt

SEQ ID NO: 104 - DNA, 100 bp, synthetic construct
  source 1..100: mol_type = other DNA
tttttttttttttttttttttttttttttttttttttttttttttttttttttttttttttttttttttttttttttttttttttttttttttttttttt

SEQ ID NO: 105 - DNA, 16 bp, synthetic construct
  source 1..16: mol_type = other DNA
taccgtcgcgacggta

SEQ ID NO: 106 - RNA, 263 nt, synthetic construct
  source 1..263: mol_type = other RNA
gggtcttcgagaagacctgttttagagctagaaatagcaagttaaaataaggctagtccgttatcaacttacgggcggtcggcctggcgtgaattctaccgtcgttgtaccgtctacttgactcgcctgtcaccggatgtgctttccggtctgatgagtccgtgaggacgaaacaggcgattttttccgtcgttgtaccgtctacttgactagggatccacgccaggccgaccgcccgtaagtggcaccgagtcggtgctttt

SEQ ID NO: 107 - RNA, 52 nt, synthetic construct
  source 1..52: mol_type = other RNA
cctgtcaccggatgtgctttccggtctgatgagtccgtgaggacgaaacagg

SEQ ID NO: 108 - RNA, 67 nt, synthetic construct
  source 1..67: mol_type = other RNA
  modified_base 6: mod_base = OTHER; note = Nucleotide with phosphorothioate linkage
ggcctcctggtatccaatccgtgaggatgtactaccagctgatgagtcccaaataggacgaaaggcc

SEQ ID NO: 109 - RNA, 69 nt, synthetic construct
  source 1..69: mol_type = other RNA
  modified_base 6: mod_base = OTHER; note = Nucleotide with phosphorothioate linkage
ggcctcctggtatccaatccgtgaggatgtactaccagctgatgagtgcccaaataggcacgaaaggcc

SEQ ID NO: 110 - RNA, 71 nt, synthetic construct
  source 1..71: mol_type = other RNA
  modified_base 6: mod_base = OTHER; note = Nucleotide with phosphorothioate linkage
ggcctcctggtatccaatccgtgaggatgtactaccagctgatgagtggcccaaataggccacgaaaggcc

SEQ ID NO: 111 - DNA, 119 bp, synthetic construct
  source 1..119: mol_type = other DNA
ggcgacgtaaagttttagagctaccgtcgttgtaccgtctacttgacttttttttcagccaactccaattttttttgtcaagtagacggtacaacgacggtagcaagttaaaataaggc

SEQ ID NO: 112 - DNA, 54 bp, synthetic construct
  source 1..54: mol_type = other DNA
ggcgacgtaaagttttagagctaccgtcgcgacggtagcaagttaaaataaggc

SEQ ID NO: 113 - DNA, 54 bp, synthetic construct
  source 1..54: mol_type = other DNA
ggcgacgtaaagttttagagctaccgtcgcgacggtagcaagtttaaataaggc

SEQ ID NO: 114 - DNA, 55 bp, synthetic construct
  source 1..55: mol_type = other DNA
ggcgacgtaaagttttagagctaccgtcgtcgacggtagcaagttaaaataaggc

SEQ ID NO: 115 - DNA, 55 bp, synthetic construct
  source 1..55: mol_type = other DNA
ggcgacgtaaagttttagagctaccgtcgacgacggtagcaagttaaaataaggc

SEQ ID NO: 116 - DNA, 54 bp, synthetic construct
  source 1..54: mol_type = other DNA
ggcgacgtaaagttttagagctaccgtcgcgacggtagctagttaaaataaggc

SEQ ID NO: 117 - DNA, 54 bp, synthetic construct
  source 1..54: mol_type = other DNA
ggcgacgtaaagttttagagctaccgtcgcgacggtagcaagataaaataaggc

SEQ ID NO: 118 - DNA, 83 bp, synthetic construct
  source 1..83: mol_type = other DNA
cgaggtcacggcgtgttttagagctacctgaaggtaaagatcgggtcctctttttttttgaggacccgatctttaccttcagg

SEQ ID NO: 119 - DNA, 82 bp, synthetic construct
  source 1..82: mol_type = other DNA
cctgaaggtaaagatcgggtcctcaaaaaaaagaggacccgatctttaccttcaggtagctctaaaacacgccgtgacctcg

SEQ ID NO: 120 - DNA, 102 bp, synthetic construct
  source 1..102: mol_type = other DNA
cgaggtcacggcgttgttttagagctacctacactcgacatcggtagctattttttttcagccaactccaatttttttttagctaccgatgtcgagtgtagg

SEQ ID NO: 121 - DNA, 102 bp, synthetic construct
  source 1..102: mol_type = other DNA
cctacactcgacatcggtagctaaaaaaaaattggagttggctgaaaaaaaatagctaccgatgtcgagtgtaggtagctctaaaacaacgccgtgacctcg

(19) United States
(12) Patent Application Publication
(10) Pub. No.: US 2025/0354153 A1
(43) Pub. Date: Nov. 20, 2025

(54) SYSTEMS FOR CELL PROGRAMMING AND METHODS THEREOF

(71) Applicant: Syntax Bio, Inc., Chicago, IL (US)

(72) Inventors: Ryan CLARKE, Chicago, IL (US); Bradley J. MERRILL, Oak Park, IL (US); Anupama PUPPALA, Chicago, IL (US); Andrew NIELSEN, Chicago, IL (US); Nikolas George Koutis BALANIS, Chicago, IL (US); Andrew P. MAY, San Francisco, CA (US)

(21) Appl. No.: 19/024,131

(22) Filed: Jan. 16, 2025

Related U.S. Application Data
(63) Continuation of application No. PCT/US23/28169, filed on Jul. 19, 2023.
(60) Provisional application No. 63/390,731, filed on Jul. 20, 2022.

(51) Int. Cl. C12N 15/113

(52) U.S. Cl. CPC C12N 15/1137; C12N 2310/531

(57) ABSTRACT

Provided herein are systems of regulating expression of a cargo (e.g., a guide nucleic acid) from a polynucleotide sequence (e.g., a vector).

### CROSS REFERENCE

This application is a continuation of International Application No. PCT/US23/28169, filed Jul. 19, 2023, which claims the benefit of U.S. Provisional Patent Application No. 63/390,731, filed on Jul. 20, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 16, 2025, is named 61684-707-301_SL.xml and is 88,137 bytes in size.

### BACKGROUND

Heterologous proteins and/or nucleic acid molecules can be utilized to elicit a desired response in a cell. The heterologous proteins and/or nucleic acid molecules can regulate genes of interest (e.g., transgenes and/or endogenous genes) to program (e.g., differentiate, de-differentiate) a cell. In some cases, endonuclease-based technologies (e.g., clustered regularly interspaced short palindromic repeats (CRISPR)-associated protein or “CRISPR/Cas”) have been adopted for manipulation of polynucleotide sequences, epigenetic modification thereof, and/or expression level thereof. For example, the CRISPR/Cas technology can be characterized by its versatility and facile programmability and can be used to promote genome editing across different species.

### SUMMARY

The present disclosure provides methods and systems for regulating expression or activity of target genes. Some aspects of the present disclosure provide methods and systems for utilizing transcription termination sequences (e.g. a polyX sequence) to control sgRNA-mediated genetic circuits which regulate the expression or activity of target genes.

In an aspect, the present disclosure provides a system for regulating expression or activity of a target gene, the system comprising: a polynucleotide sequence encoding a guide nucleic acid molecule, wherein the guide nucleic acid molecule exhibits specific affinity to the target gene, to regulate the expression or the activity of the target gene, wherein the polynucleotide sequence comprises a domain that (i) corresponds to a tetraloop region of the guide nucleic acid molecule, and (ii) comprises a polyT sequence, wherein the polyT sequence is sufficient to reduce expression of the guide nucleic acid molecule, thereby regulating expression or activity of the target gene.

In another aspect, the present disclosure provides a system for regulating expression or activity of a target gene, the system comprising: a polynucleotide sequence encoding a guide nucleic acid molecule, wherein the guide nucleic acid molecule is characterized by (i) exhibiting specific affinity to the target gene, to regulate the expression or activity of the target gene, and (ii) has a size of at least about 12 nucleotides, wherein the polynucleotide sequence comprises a polyX sequence having a threshold length that is greater than or equal to five, such that the poly X sequence is sufficient to reduce expression of the guide nucleic acid molecule from the polynucleotide sequence, wherein the polyX sequence does not correspond to a terminal domain of the guide nucleic acid molecule.

In another aspect, the present disclosure provides a method for regulating expression or activity of a target gene in a cell, the system comprising: contacting the cell with a polynucleotide sequence encoding a guide nucleic acid molecule, wherein the guide nucleic acid molecule exhibits specific affinity to the target gene, to regulate the expression or the activity of the target gene, wherein the polynucleotide sequence comprises a domain that (i) corresponds to a tetraloop region of the guide nucleic acid molecule, and (ii) comprises a polyT sequence, wherein the polyT sequence is sufficient to reduce expression of the guide nucleic acid molecule, thereby regulating expression or activity of the target gene.

In another aspect, the present disclosure provides a method for regulating expression or activity of a target gene in a cell, the method comprising: providing a polynucleotide sequence encoding a guide nucleic acid molecule to the cell, wherein the guide nucleic acid molecule is characterized by (i) exhibiting specific affinity to the target gene, to regulate the expression or activity of the target gene, and (ii) has a size of at least about 12 nucleotides, wherein the polynucleotide sequence comprises a polyX sequence having a threshold length that is greater than or equal to five, such that the polyX sequence is sufficient to reduce expression of the guide nucleic acid molecule from the polynucleotide sequence, wherein the polyX sequence does not correspond to a terminal domain of the guide nucleic acid molecule.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows an example of a sgRNA with a ribozyme. FIG. 1B shows another example of a sgRNA with a ribozyme. Figure discloses SEQ ID NO: 106.

FIGS. 2A-2D show elongation modifications of ribozymal structures of sgRNA. FIG. 2A shows a minimal hammerhead ribozyme. Figure discloses SEQ ID NO: 107. FIG. 2B shows a 4-bp long stem II. Figure discloses SEQ ID NO: 108. FIG. 2C shows a 5-bp long stem II. Figure discloses SEQ ID NO: 109. FIG. 2D shows a 6-bp long stem II. Figure discloses SEQ ID NO: 110.

FIG. 2E shows how elongation of the stem II loop on a ribozymes hinders ribozyme activity.

FIG. 3 depicts the results of testing various sgRNA modifications for the ability to deactivate the guide nucleic acid.

FIG. 4A-4B illustrate how longer polyT sequences are correlated with increased termination efficiency. FIG. 4A shows different hairpin polyT sequence variants. FIG. 4B shows different tetraloop polyT sequence variants. FIG. 4C shows termination efficiency as compared to the length of the polyT sequence.

FIG. 5A shows different insulator variants able to be used with sgRNAs. FIGS. 5B-5C shows that various polyU guide RNAs with variant insulators approach sgRNA-level activity using tetraloop PolyU guides (FIG. 5B) and hairpin PolyU guides (FIG. 5C). FIG. 5D demonstrates the stabilization of different guide RNAs and how they compare to unmodified sgRNA. In FIG. 5D, Panel A, the insulator region prior to the polyU region in the unmodified guide allows for the mature, modified guide to resemble the sgRNA, stabilizing the mature guide. In FIG. 5D, Panel B, the lack of an insulator region causes the mature, modified guide to be less similar to the sgRNA, destabilizing the mature guide.

FIGS. 6A-6B show that gRNAs developed with the misfolding module as the inactivating element when using tetraloop ribozymes (FIG. 6A) and tetraloop PolyU sequences (FIG. 6B).

FIG. 7 depicts the structure of a readthrough proGuide transcript (e.g, wherein the polyT fails to terminate RNA PolIII transcription) for a proGuide with an Insulator (I) structure.

FIG. 8 depicts the structure of a readthrough proGuide transcript (e.g, wherein the polyT fails to terminate RNA PolIII transcription) for a proGuide with an Insulator-Stem (IS) structure.

FIG. 9 shows dCas9 GFP disruption across variant sgRNA modifications.

FIGS. 10A-10B show that gRNA efficiency reaches a maximum cap threshold both when looking at variant sgRNA modifications (FIG. 10A) and when looking at the percent of gRNA (denoted as PG) (FIG. 10B).

FIG. 11 shows that there is minimal effect of insulator sequences on sgRNA activity.

FIG. 12 shows an example of a non-canonical terminator sequence in the non-disrupted state (Panel A) and the disrupted state (Panel B).

FIG. 13 is a schematic of the heterologous genetic circuit. An activating moiety initiates the circuit and can activate a gate unit. A gate unit can be comprised of a gate moiety and/or a gene regulating moiety.

FIG. 14 shows that the sgRNA, not the ribozyme, acts as the regulatory unit on the tetraloop.

FIGS. 15A-15E depict a 10-Step Forward Cascade at 12 hours (FIG. 15A), 24 hours (FIG. 15B), 36 hours (FIG. 15C), 48 hours (FIG. 15D), 72 hours (FIG. 15E).

FIGS. 16A-16E depict a 10-Step Reverse Cascade at 12 hours (FIG. 16A), 24 hours (FIG. 16B), 36 hours (FIG. 16C), 48 hours (FIG. 16D), 72 hours (FIG. 16E).

FIG. 17A depicts a 10-Step Forward Cascade from 0 to 48 hours.

FIG. 17B depicts a 10-Step Forward Cascade from 0 to 72 hours.

FIG. 17C depicts a 10-Step Reverse Cascade from 0 to 48 hours.

FIG. 17D depicts a 10-Step Reverse Cascade from 0 to 72 hours.

FIG. 18 shows the 10-Step Reverse Cascade (at Step 9) and the old stem cascade (at Step 4) compared to endogenous.

FIG. 19 shows a comparison of single polyT, linear multipoly T, 5S RNA multipolyT against untransfected and sgRNA controls on the performance of transcriptional termination in proGuides.

FIG. 20A shows a frequency of RNA corresponding to a perfect NHEJ repair outcome for a Type 3 proGuide.

FIG. 20B shows the DNA sequences observed from the experiment for the Type 3 proGuide in FIG. 20A. Figure discloses SEQ ID NOS 111-112, 112, 112, 112-113, 112, 114, 112, 112, 115-117, 114, 117, and 112, respectively, in order of appearance.

FIG. 21A shows the size distribution of mapped sequencing reads for Type 1 proGuide. Perfect NHEJ repair outcome is denoted by an arrow (e.g. 166 nt length of matureGuide RNA) and the triangle denotes the length of the proGuide RNA (e.g. 254 nt).

FIG. 21B shows the size distribution of mapped sequencing reads for Type 2 proGuide. Perfect NHEJ repair outcome is denoted by an arrow (e.g. 97 nt length of matureGuide RNA) and the triangle denotes the length of the proGuide RNA (e.g. 162 nt).

FIG. 21C shows the size distribution of mapped sequencing reads for Type 3 proGuide. Perfect NHEJ repair outcome is denoted by an arrow (e.g. 97 nt length of matureGuide RNA) and the triangle denotes the length of the proGuide RNA (e.g. 162 nt).

FIG. 21D shows the size distribution of mapped sequencing reads for Type 3 proGuide with a less than optimal cut site (e.g. APC) compared to FIG. 21C (e.g. Axin1). Perfect NHEJ repair outcome is denoted by an arrow (e.g. 97 nt length of matureGuide RNA) and the triangle denotes the length of the proGuide RNA (e.g. 162 nt).

FIG. 22A depicts an example architecture of a Gen2 proGuide Unit including a single polyT (e.g. 9 nt) sequence. Figure discloses SEQ ID NOS 118-119, respectively, in order of appearance.

FIG. 22B depicts an example architecture of a Gen3 proGuide Unit including multiple (e.g.) polyT sequences separated by a linear sequence. Figure discloses SEQ ID NOS 120-121, respectively, in order of appearance.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise. For example, the term “a gate unit” includes a plurality of gate units.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “guide nucleic acid,” “guide nucleic acid molecule,” and “gNA” as used interchangeably herein, generally refer to 1) a guide sequence that can hybridize to a target sequence or 2) a scaffold sequence that can interact with or complex with a nucleic acid guide nuclease. A guide nucleic acid can be a single-guide nucleic acid (e.g., sgRNA) or a double-guide nucleic acid (e.g., dgRNA). sgRNA can be a single RNA molecule that contains both a scaffold tracrRNA and a crRNA which can be complementary to the target sequence. Alternatively, dgRNA can be a single RNA molecule that contains a crRNA annealed to a tracrRNA through a direct repeat sequence.

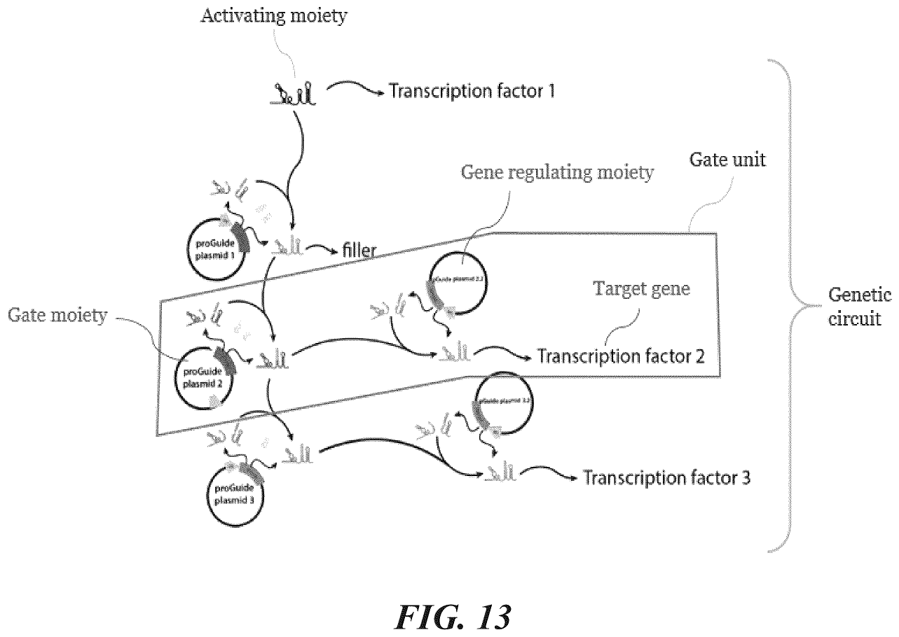

The term “genetic circuit,” “biological circuit,” or “circuit,” as used interchangeably herein, generally refers to a collection of molecular components (e.g., biological materials, such as polypeptides and/or polynucleotides, non-biological materials, etc.) operatively coupled (e.g., operating simultaneously, sequentially, etc.) accordingly to a circuit design. The collection of the molecular components can be capable of providing one or more specific outputs in a cell (e.g., regulation of one or more genes) in response to one or more inputs (e.g., a single input or a plurality of inputs). Such one or more inputs can be sufficient to trigger the molecular components of the genetic circuit to provide the one or more specific outputs. For example, the genetic circuit can comprise one or more molecular switches that are activatable by one or more inputs (FIG. 13).

A genetic circuit can be a controllable gene expression system comprising an assembly of biological parts that work together (e.g., simultaneously, sequentially, etc.) as a logical function. A genetic circuit can comprise a plurality of gate units, wherein at least one gate unit of the plurality of gate units can be activatable by an activating moiety (e.g., a heterologous input to the cell) to activate other gate units of the plurality of gate units (e.g., simultaneously at once, sequentially in a cascading manner, etc.) (FIG. 13). For example, at least one gate unit of the plurality of gate units can be activatable (e.g., directly or indirectly) by another gate unit of the plurality of gate units, to (i) regulate expression or activity level of one or more target genes, (ii) activate at least one another gate unit of the plurality of gate units, and/or (ii) deactivate at least one another gate unit of the plurality of gate units, thereby collectively regulating expression and/or activity level of one or more target genes in a desired manner, as predetermined by the design of the genetic circuit (FIG. 13). The terms “heterologous genetic circuit,” “HGC,” “cellular algorithm,” or “cellgorithm” as used herein may be used interchangeably.

The term “gate unit,” as referred to herein, generally refers to a portion of the genetic circuit that can control gene regulation by functioning similarly to a logic gate wherein it can control the flow of information and allow the circuit to multiplex decision making at different points. More specifically, the term refers to a nucleic acid encoding a genetic switch and a transcription and/or translation regulatory region, or series of regions, which the genetic switch acts on. The input for a gate unit can be an activating moiety and/or another gate unit. The output for a gate unit can be used to activate another gate unit, to de-activate another gate unit, to affect a target gene, and/or a combination of any of the above. For example, a gate unit can be comprised of a plurality of gate moieties and/or a plurality of gene regulating moieties (FIG. 13).

The term “activating moiety,” as referred to herein, generally refers to a moiety that can activate plurality of genetic circuits and/or a plurality of gate units. An activating moiety can be a heterologous input to a cell. In some cases, activating moieties can include, but are not limited to, a guide nucleic acid molecule (e.g., a gRNA) or other nucleic acid, polypeptides, polynucleotides, small molecules, light, or a combination thereof. For example, an activating moiety can be a guide nucleic acid molecule that forms a complex with an endonuclease (e.g., a Cas protein) to bind to a polynucleotide sequence of a gate moiety (e.g., a plasmid encoding another guide nucleic acid molecule) that is inactivated, to activate such gate moiety (e.g., induce expression of a functional form of the additional guide nucleic acid molecule) that can target one or more gene regulating moieties.

The term “gate moiety,” as referred to herein, generally refers to a moiety that can affect the function of a gene regulating moiety within a gate unit. A gate moiety can activate and/or deactivate a gene regulating moiety. For example, a gate moiety can regulate expression of a gene regulation moiety by editing a nucleic acid sequence and thereby activating or deactivating the gene regulating moiety. For example, a gate moiety can be a guide nucleic acid molecule that forms a complex with an endonuclease (e.g., a Cas protein) to bind to a polynucleotide sequence of a gene regulating moiety (e.g., a plasmid encoding another guide nucleic acid molecule) to activate the gene regulating moiety (e.g., induce expression of a functional form of the another guide nucleic acid molecule) that can target one or more endogenous genes of a cell. Alternatively or in addition to, a gate moiety can activate and/or deactivate another gate unit of the genetic circuit (FIG. 13). For example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is inactivated, to activate the another gate moiety (e.g., induce expression of a functional form of the another guide nucleic acid molecule). In another example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is activated, to inactivate the another gate moiety (e.g., reduce expression of a functional form of the another guide nucleic acid molecule).

The term “gene regulating moiety” or “gene editing moiety” as used interchangeably herein, generally refers to a moiety which can regulate the expression and or activity profile of a nucleic acid sequence or protein, whether exogenous or endogenous to a cell (FIG. 13). For example, a gene editing moiety can regulate expression of a gene by editing a nucleic acid sequence (e.g. CRISPR-Cas, Zinc-finger nucleases, TALENs, or siRNA). In some cases, a gene editing moiety can regulate expression of a gene by editing a genomic DNA sequence. In some cases, a gene editing moiety can regulate expression of a gene by editing an mRNA template. Editing a nucleic acid sequence can, in some cases, alter the underlying template for gene expression (e.g. CRISPR-Cas-inspired RNA targeting systems). Alternatively, a gene editing moiety can repress translation of a gene (e.g. Cas13).

Alternatively or in addition to, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. For example, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript. In some cases, a gene editing moiety can regulate a gene through epigenetic editing (e.g. Cas12).

In some cases, a plasmid can encode a non-functional form of a gene editing moiety. The plasmid can be activated (e.g., genetically modified) to express a functional form of the gene editing moiety, e.g., via activation of a functional gate moiety. For example, the plasmid can encode a non-functional form of a guide nucleic acid molecule that would otherwise be able to bind to a target gene of a cell. Upon binding of a functional gate moiety (e.g., another guide nucleic acid molecule complexed with a Cas protein) to the plasmid, the plasmid can be edited (e.g., cleaved at one or more sites, then repaired via endogenous mechanisms (e.g., homologous recombination, nonhomologous end joining) to allow expression of a functional form of the gene editing moiety (e.g., a functional form of the guide nucleic acid molecule with specific binding to the target gene of the cell), to permit modulation of the target gene in the cell.

In some cases, a gene regulating moiety can comprise a nucleic acid molecule (e.g., a guide nucleic acid molecule that forms a complex with an endonuclease, such as a Cas protein). Alternatively or in addition to, a gene regulating moiety can comprise or be operatively coupled to an endonuclease. An endonuclease can be an enzyme that cleaves a phosphodiester bond within a polynucleotide chain. An endonuclease can comprise restriction endonucleases that cleave DNA at specific sites without damaging bases. Restriction endonucleases can include Type I, Type II, Type III, and Type IV endonucleases, which can further include subtypes. In some cases, an endonuclease can be Cas1, Cas2, Cas 3, Cas4, Cas5, Cas6, Cas7, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas 13 (C2c2), Cas13b, Cas13c, Cas13d, Cas13x.1, Cse1, Cse2, Csy1, Csy2, Csy3, Csm2, Cmr5, Csx10, Csx11, Csf1, Csn2. An endonuclease can be a dead endonuclease which exhibits reduced cleavage activity. For example, an endonuclease can be a nuclease inactivated Cas such as a dCas (e.g., dCas9).

The abovementioned Cas proteins can form a complex with a guide nucleic acid (gNA (e.g., a guide RNA (gRNA)) and utilize the gNA to specifically bind to a target polynucleotide sequence (e.g., a target DNA sequence, a target RNA sequence). Accordingly, in some cases, such Cas proteins may be referred to as a “NA-guided nuclease” (e.g., RNA-guided nuclease). As used herein, the term “guide nucleic acid” (gNA) can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence”. A nucleic acid-targeting segment may comprise a sub-segment that may be referred to as a “protein binding segment” or “protein binding sequence” or “Cas protein binding segment” or “scaffold sequence.”

A gene regulating moiety can be a transcriptional modulator system (e.g., a gene repressor complex or a gene activator complex). For example, a gene regulating moiety can be a gene repressor complex comprising a dCas protein operatively coupled to (e.g., coupled to or fused with) a transcriptional repressor. Non-limiting examples of transcriptional repressors can include KRAB, SID, MBD2, MBD3, DNMT1, DNMT2A, DNMT3A, DNMT3B, DNMT3L, Mecp2, FOG1, ROM2, LSD1, ERD, SRDX repression domain, Pr-SET7/8, SUV4-20H1, RIZ1, JMJD2A, JHDM3A, JMJD2B, JMJD2C, GASC1, JMJD2D, JARID1A, RBP2, JARID1B/PLU-1, JARIDIC/SMCX, JARIDID/SMCY, HDAC1, HDAC2, HDAC3, HDAC8, HDAC4, HDAC5, HDAC7, HDAC9, SIRT1, SIRT2, HDAC11, M.Hhal, METI, DRM3, ZMET2, CMT1, CMT2, Lamin A, and Lamin B. Alternatively, a gene regulating moiety can be a gene activator complex comprising a dCas protein operatively coupled to (e.g., fused to) a transcriptional activator. Non-limiting examples of transcriptional activators can include VP16, VP64, VP48, VP160, p65 subdomain, SETIA, SET1B, MLL1, MLL2, MLL3, MLL4, MLL5, ASH1, SYMD2, NSD1, JHDM2a, JHDM2b, UTX, JMJD3, GCN5, PCAF, CBP, p300, TAF1, TIP60/PLIP, MOZ/MYST3, MORF/MYST4, SRCl, ACTR, P160, CLOCK, TET1CD, TET1, DME, DML1, DML2, and ROS1.

In some cases, the gene regulating moiety has enzymatic activity that modifies the target gene without cleaving the target gene. Modification of the target gene can cause, for example, epigenetic modifications that can modify gene expression and/or activity level. Examples of enzymatic activity that can be provided by a gene regulating moiety can include but are not limited to: nuclease activity such as that provided by a restriction enzyme (e.g., Fokl nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3, ZMET2, CMT1, CMT2; demethylase activity such as that provided by a demethylase (e.g., Ten-Eleven Translocation (TET) dioxygenase 1 (TET1CD), TET1, DME, DML1, DML2, ROS 1), DNA repair activity, DNA damage activity, deamination activity such as that provided by a deaminase (e.g., a cytosine deaminase enzyme such as APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity.

Unless specifically stated or obvious from context, the term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleotide). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. During transient expression, episomal DNA can be transferred to daughter cells, but since episomal DNA is not replicated, it is not permanently heritable and will dilute out over time. In contrast, stable expression of a transfected gene can occur when the gene is co-transfected with another gene that confers a selection advantage to the transfected cell. During stable expression, plasmids can have a DNA replication element that allows them to be inherited or integrated into the genome. Such a selection advantage may be a resistance towards a certain toxin that is presented to the cell.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used interchangeably herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

Unless specifically stated or obvious from context, the term “nucleotide” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [aS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G] dUTP, [TAMRA] dUTP, [R110] dCTP, [R6G] dCTP, [TAMRA] dCTP, [JOE] ddATP, [R6G] ddATP, [FAM] ddCTP, [R110] ddCTP, [TAMRA] ddGTP, [ROX] ddTTP, [dR6G] ddATP, [dR110] ddCTP, [dTAMRA] ddGTP, and [dROX] ddTTP available from Perkin Elmer, Foster City, Calif. FluoroLink Deoxy Nucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR 770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

### OVERVIEW

Biological programming, such as cellular programming, allows for the engineering of a cell to generate a desired outcome. Outcomes of cellular programming can include inducing or prevent a wide array of common and/or new cellular functions; outcomes can also include enhancing or repressing an already-occurring cellular function. Cellular programming can be accomplished through the use of a genetic circuit. Cellular programming can be accomplished through the manipulation of biomolecules (e.g., DNA). For example, CRISPR or CRISPR/Cas systems have been adopted for genome editing across many species due to its versatility and facile programmability. Cellular programming can affect endogenous or exogenous genes. Cellular programming can be implemented to function in a time-dependent manner or a time-independent manner.

Genetic circuits used in cellular programming can be used to control a cascade of a plurality of desired expression and/or activity profiles of a plurality of genes in the cell. To allow for better control of specific cellular outcomes, genetic circuits can be multiplexed to create positive feedback and/or negative feedback systems.

Although CRISPR/Cas systems are widely used for gene editing, Cas can be a single-turnover nuclease as it remains bound to the double-strand break it generates, and many regions of the genome are refractory to genome editing. Increased understanding of CRISPR/Cas-based genome editing has encouraged the development of cascading regulatory systems to further harness this technology for use in engineered cellular development. By implementing a series of activatable gRNA, genome editing can be regulated from target site to target site in more of a temporal manner, sequential genome edits can be executed to function like a domino effect, and cells can be barcoded. However, this barcoding doesn't enable epigenetic gene regulations that can be employed for cellular differentiations.

Thus, there remains an unmet need for an activatable, multiplexed CRISPR/Cas system and use of the same to edit a target polynucleotide (e.g., a genome of a cell, in particular a eukaryotic cell), using cascades of gRNAs to form genetic circuits which include feedback loops in order to single-handedly affect gene regulation and, in turn, cell-fate determination. Given its improved multiplexing capabilities through the use of internal positive and/or negative feedback loops, the preprogrammed, activatable, and self-regulating gRNA cascade CRISPR/Cas system finds use, e.g., in gene therapy, genetic circuitry, and/or complex cell-fate determination and/or control.

Thus, the present disclosure provides systems, compositions, and methods thereof for controlling a gene regulating moiety (e.g., a guide nucleic acid molecule of a CRISPR/Cas system), such that the activity of the gene regulating moiety to effect regulation of one or more target genes (e.g., in a cell) can be controlled. In some embodiments, controlling of the gene regulating moiety can comprise controlling expression or activity level of the gene regulating moiety. In some embodiments, the present disclosure provides systems, compositions, and methods for controlling activity of a CRISPR/Cas system (e.g., a CRISPR/Cas9 system), comprising a Cas endonuclease and one or an array of cognate single guide RNAs (sgRNA or gRNA) that (i) harbor inactivation sequences in a non-essential region and (ii) are activatable, to allow for modulation and modification of that system.

### Systems and Method for Activating and Deactivating Guide Nucleic Acids

Various aspects of the present disclosure provides systems and methods for controlling expression of a molecule of interest (e.g., a polynucleotide molecule) from a polynucleotide sequence encoding the molecule of interest. In some embodiments, the polynucleotide sequence can be a vector or an expression cassette encoding the polynucleotide sequence that encodes the molecule of interest. For example, the polynucleotide sequence can be a DNA sequence, and the expression can be transcription of at least a portion of the DNA sequence to a RNA sequence. As provided herein, the molecule of interest, once expressed, can be utilized as a therapeutic molecule. In some cases, the expressed variant of the molecule of interest can exhibit specific binding to a target gene for regulation (or modulation) of expression or epigenetic profile of the target gene. For example, the molecule of interest can be at least a portion of (e.g., partial or full) shRNA or a guide nucleic acid molecule to form a complex with an endonuclease (e.g., Cas protein).

A domain of the polynucleotide sequence that encodes (or corresponds to) the molecule of interest can comprise a polyX sequence. The polyX sequence can be sufficient to reduce expression of the molecule of interest (e.g., the guide nucleic acid molecule) from the polynucleotide sequence. For example, the polyX sequence can be disposed within the domain encoding the molecule of interest (e.g., not at either the 5′ end or the 3′ end of such domain), such that expression of the molecule of interest (e.g., transcription of an RNA molecule of interest) would be disrupted (e.g., terminated) in the middle of the expression.

Accordingly, the polyX sequence (e.g., in the polynucleotide sequence encoding the molecule of interest) may be referred to as a termination sequence (e.g., a non-canonical termination sequence for its sequence and/or its position), as a disruption sequence (e.g., for disruption of full expression of the molecule of interest), as an inactivation sequence (e.g., for inactivating function of the polynucleotide sequence or the molecule of interest).

As provided herein, the molecule of interest can be a guide nucleic acid molecule that, when expressed in an active or functional state, comprises a spacer region (e.g., for binding a target gene) and a scaffold region (e.g., for complexing with a Cas protein). In the domain of the polynucleotide sequence that encodes the guide nucleic acid molecule of interest, the polyX can be disposed within the spacer region-encoding sequence, disposed between the spacer region-encoding sequence and the scaffold-encoding sequence, and/or disposed within the scaffold encoding sequence. In some cases, the scaffold region can comprise one or more loops (e.g., formed by two polynucleotide segments that are partially or entirely complementary to one another)), such as, for example, a tetraloop and one or more stem loops. In some cases, the polyX can be disposed at, adjacent to, or within a portion of the polynucleotide sequence that encodes the one or more loops.

In some cases, the polynucleotide sequence can be described for having the poly X sequence.

In some cases, the molecule of interest that is encoded by the polynucleotide sequence can be described for having the polyX sequence. In some examples, description of the molecule of interest (e.g., a guide nucleic acid molecule) having the polyX sequence may be referring to the expressed (e.g., transcribed) form of the molecule of interest. Alternatively or in addition to, description of the molecule of interest having the polyX sequence may be referring to the polynucleotide sequence that encodes such molecule of interest.

Accordingly, additional aspects of the present disclosure provides systems and methods for modifying (e.g., via mutation, via partial or complete removal, etc.) such polyX sequence within the polynucleotide sequence, thereby activating the polynucleotide sequence (e.g., to express a the molecule of interest in an active/functional state) or activating the molecule of interest (e.g., to be expressed in such active/functional state).

In some cases, the tetraloop domain can be a polyX sequence. A polyX sequence can be a polyA sequence, a polyG sequence, a polyC sequence, a polyT sequence, or a polyU sequence. In some cases, the polyX sequence can be a polyT sequence. A polyX sequence can cause premature termination. In some cases, a polyT sequence can cause premature termination. In eukaryotic cells, RNA polymerase III (Pol III) is a protein that can transcribe DNA to synthesize small noncoding ribosomal nucleic acids. Termination of Pol III-controlled transcription can occur at stretches of polyT sequences at the end of a gene.

In some cases, the polyX sequence can be located within (e.g., not at a terminal end) a polynucleotide sequence, such as a DNA sequence or an RNA sequence. In some cases, the poly X sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 3′ end of the polynucleotide sequence. In some cases, the poly X sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 5′ end of the polynucleotide sequence. In some cases, the polyX sequence can be located at a terminal end of a nucleic acid sequence.

In some cases, the polyT or polyU sequence can be located within (e.g., not at a terminal end) a polynucleotide sequence, such as a DNA sequence or an RNA sequence. In some cases, the polyT or polyU sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 3′ end of the polynucleotide sequence. In some cases, the polyT or polyU sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 5′ end of the polynucleotide sequence. In some cases, the polyT or polyU sequence can be located at a terminal end of a nucleic acid sequence. In some cases, an RNA which comprises a polyU sequence can also be represented by a DNA which comprises a polyT sequence.

A poly X sequence (e.g., a polyT sequence or a polyU sequence) can comprise at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, at least about 50 X, at least about 60, at least about 70, at least about 80, at least about 90, or at least about 100 bases. A polyX sequence can comprise at most about 100, at most about 90, at most about 80, at most about 70, at most about 60, at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, or at most about 2 X bases. A polyX sequence can be represented by a complementary poly X sequence in a corresponding complementary DNA strand (e.g., a polyT, as disclosed herein as a DNA sequence, can also be referred to as polyA in the complementary DNA strand). The polyX sequence as disclosed can comprise a plurality of X bases. The plurality of X bases can be disclosed sequentially adjacent to one another (e.g., TT, TTT, TTTT, TTTTT, etc.). Alternatively or in addition to, the plurality of X bases can be separated by one or more additional nucleotides that are not X. The one or more additional nucleotides can comprise a single type of nucleotide or different types of nucleotides.

In some cases, a polyX sequence (e.g., a polyT sequence) can comprise a consecutive sequence of identical X nucleobases (e.g., identical T nucleobases). Such consecutive sequence can comprise at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, at least or up to about 30, at least or up to about 35, at least or up to about 40, at least or up to about 45, or at least or up to about 50 identical X nucleobases (e.g., such consecutive number of T bases, such consecutive number of U bases, etc.).

In some cases, the one or more additional nucleotides that are not X can be flanked by by (or disposed between) (i) one or more 5′ X bases and (ii) one or more 3′ X bases. In some cases, the region flanked by the 5′ X bases and the 3′ X bases can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, or at least about 50 bases in length. In some cases, the region flanked by the 5′ X bases and the 3′ X bases can be at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 bases in length. For example, see the structure (I) as discussed below.

In some cases, one or more X sequences can flank either the 5′ and/or the 3′ end of the one or more additional nucleotides that are not X. In some cases, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, or at least about 50 X sequences can be 5′ of the one or more additional nucleotides that are not X. In some cases, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, or at least about 50 X sequences can be 3′ of the one or more additional nucleotides that are not X. In some cases, at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 X sequences can be 5′ of the one or more additional nucleotides that are not X. In some cases, at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 X sequences can be 3′ of the one or more additional nucleotides that are not X.

In some cases, there can be a number of non-X additional nucleotides greater than the number of X nucleotides (e.g., within the tetraloop domain comprising the poly X sequence). For example, there can be a number of non-U additional nucleotides greater than the number of U nucleotides within the tetraloop domain of an RNA comprising a polyU sequence. In some cases, there can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, or at least about 50 more non-X additional nucleotides than there are X nucleotides. In some cases, there can be an equal number of non-X additional nucleotides as there are X nucleotides. In some cases, there can be a number of non-X additional nucleotides less than the number of X nucleotides. In some cases, there can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, or at least about 50 fewer non-X additional nucleotides as there are X nucleotides.

A polyX sequence can be at least 2, at least 3, at least 4, at least 5, at least 6, at least 7, at least 8, at least 9, at least 10, at least 11, at least 12, at least 13, at least 14, at least 15, at least 20, at least 30, at least 40, at least 50 X, at least 60, at least 70, at least 80, at least 90, or at least 100 X bases in length. A polyX sequence can be at most about 100, at most about 90, at most about 80, at most about 70, at most about 60, at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, or at most about 2 X bases in length. A polyX sequence can be represented by a corresponding polyX sequence in a corresponding RNA. For example, a polyT sequence can be represented by a corresponding polyU sequence in a corresponding RNA. A polyX sequence can be between about 4 and 8, between about 4 and 10, between about 5 and 7, between about 5 and 8, between about 5 and 10, between about 5 and 15, between about 6 and 8, between about 6 and 10, between about 6 and 15, or between about 7 and 15 T bases in length.

A polyT sequence can be at least 2, at least 3, at least 4, at least 5, at least 6, at least 7, at least 8, at least 9, at least 10 (SEQ ID NO: 90), at least 11 (SEQ ID NO: 91), at least 12 (SEQ ID NO: 92), at least 13 (SEQ ID NO: 93), at least 14 (SEQ ID NO: 94), at least 15 (SEQ ID NO: 95), at least 20 (SEQ ID NO: 96), at least 30 (SEQ ID NO: 97), at least 40 (SEQ ID NO: 98), at least 50 X (SEQ ID NO: 99), at least 60 (SEQ ID NO: 100), at least 70 (SEQ ID NO: 101), at least 80 (SEQ ID NO: 102), at least 90 (SEQ ID NO: 103), or at least 100 (SEQ ID NO: 104) T bases in length. A polyT sequence can be at most about 100, at most about 90, at most about 80, at most about 70, at most about 60, at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, or at most about 2 T bases in length. A polyT sequence can be represented by a polyU sequence in a corresponding RNA. A polyT sequence can be between about 4 and 8, between about 4 and 10, between about 5 and 7, between about 5 and 8, between about 5 and 10, between about 5 and 15, between about 6 and 8, between about 6 and 10, between about 6 and 15, or between about 7 and 15 T bases in length.

In some cases, a threshold length of a polyX sequence can be necessary to effect premature termination. A threshold length of a polyX sequence can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 nucleotides in length. In some cases, a polyX sequence can be sufficient to reduce expression of a gNA molecule when compared to a control which does not have a polyX sequence. In some cases, a polyX sequence can be sufficient to reduce expression of a gNA molecule when compared to a control which has a polyX sequence which has a length shorter than that of the threshold poly X sequence.

In some cases, a threshold length of a polyT sequence can be necessary to effect premature termination. A threshold length of a polyT sequence can be at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 T. In some cases, a polyT sequence can be sufficient to reduce expression of a gNA molecule when compared to a control which does not have a polyT sequence. In some cases, a polyT sequence can be sufficient to reduce expression of a gNA molecule when compared to a control which has a polyT sequence which has a length shorter than that of the threshold polyT sequence.

As provided herein, the polyX sequence can be utilized to control activation/deactivation of a guide nucleic acid molecule. Accordingly, various aspects of the present disclosure provide systems for efficient deactivation and/or activation of guide nucleic acids (e.g., sgRNA) to allow for control over an engineered CRISPR/Cas system designed to regulation the expression or activity of a target gene. Various aspects of the present disclosure provide methods for efficient deactivation and/or activation of guide nucleic acids (e.g., sgRNA) to allow for control over an engineered CRISPR/Cas system designed to regulate the expression or activity of a target gene.

In an aspect, the present disclosure provides for a system that induces a desired expression and/or activity profile of a target gene in a cell. The system can comprise a heterologous genetic circuit comprising a plurality of gate units. The plurality of gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more gate unit(s).d The plurality of gate units can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). The plurality of gate units can be different (e.g., comprising different polynucleotide sequences).

A heterologous genetic circuit as disclosed herein can operate with a plurality of gate units in series (e.g., the plurality of gate units are connected sequentially in an end-to-end manner forming a single path), in parallel (e.g., the plurality of gate units are connected across one another, forming, for example, two or more parallel paths), or a combination thereof. In some embodiments, the plurality of gate units in series can operate in a forward cascade. In some embodiments, the forward manner can follow a numerically increasing step order (e.g. step 1 to step 2 to step 3 to step 4 to step 5, etc). In some embodiments, the plurality of gate units in series can operate in a reverse cascade. In some embodiments, the reverse cascade can follow a numerically decreasing step order (e.g. step 10 to step 9 to step 8 to step 7 to step 6, etc). In some embodiments, the plurality of gate units in series can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). In some embodiments, the plurality of gate units in series can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). A plurality of gate units as disclosed herein can operate (e.g., as predetermined by the design of the heterologous genetic circuit) in concert to induce an outcome in a cell. The outcome in the cell can comprise cell function (e.g., movement, reproduction; response to external stimuli, nutritional output, excretion, respiration, growth) and/or cell state (e.g., cell fate, differentiation, quiescence, programmed cell death). Such outcomes can be ascertained in vitro, ex vivo, and/or in vivo. For example, an outcome as disclosed herein can be ascertained in vitro by (i) measuring expression level of a gene of interest by polymerase chain reaction (PCR) or Western blotting, (ii) staining via small molecules or antibodies, (iii) cell sorting based on cell size, morphology and/or surface protein expression, (iv) using assays (e.g. cell proliferation assays, metabolic activity assays, cell killing assays) to measure phenotypic differentiation and cellular function, (v) microscopy, and/or (iv) screening for molecular and/or genetic differences using e.g., metabolomics, genomics, proteomics, lipidomics, epigenomics, and/or transcriptomics.

The heterologous genetic circuit can comprise a plurality of gate units that are sequentially activated, e.g., activated in series one after another. The plurality of gate units can comprise a functional gate unit that is preconfigured such that it is activated to regulate (e.g., directly regulate) expression and/or epigenetic profile of a target gene (e.g., an endogenous targe gene). The plurality of gate units can further comprise one or more additional gate units that are preconfigured (i) to be activated prior to the functional gate unit and (ii) to effect a subsequent activation of the functional gate unit. In some cases, the one or more additional gate units can be preconfigured to be activated to regulate one or more additional target genes. Alternatively, the one or more additional gate units may not be preconfigured to regulate any target gene (e.g., any endogenous target gene) when activated. Such one or more additional gate units may instead serve to delay (e.g., in terms of time) activation of the functional gate unit during operation of the heterologous genetic circuit, thereby delaying the expression and/or epigenetic profile of the target gene of the functional gate unit, and thus the one or more additional gate units may be referred to as “blank” gate unit(s). The heterologous genetic circuit can comprise at least or up to about 1 blank gate unit, at least or up to about 2 blank gate units, at least or up to about 3 blank gate units, at least or up to about 4 blank gate units, at least or up to about 5 blank gate units, at least or up to about 6 blank gate units, at least or up to about 7 blank gate units, at least or up to about 8 blank gate units, at least or up to about 9 blank gate units, at least or up to about 10 blank gate units, at least or up to about 11 blank gate units, at least or up to about 12 blank gate units, at least or up to about 13 blank gate units, at least or up to about 14 blank gate units, at least or up to about 15 blank gate units, at least or up to about 16 blank gate units, at least or up to about 27 blank gate units, at least or up to about 18 blank gate units, at least or up to about 19 blank gate units, at least or up to about 20 blank gate units, at least or up to about 25 blank gate units, at least or up to about 30 blank gate units, at least or up to about 35 blank gate units, at least or up to about 40 blank gate units, at least or up to about 45 blank gate units, at least or up to about 50 blank gate units.

In some cases, use of the one or more blank gate units can delay activation of the functional gate unit (e.g., as ascertained by measurement of expression/epigenetic profile of the target gene, or as ascertained by measurement of expression of a functional variant or transcribed product of the functional gate unit) by at least or up to about 1 minute, at least or up to about 5 minutes, at least or up to about 10 minutes, at least or up to about 30 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 13 hours, at least or up to about 14 hours, at least or up to about 15 hours, at least or up to about 16 hours, at least or up to about 17 hours, at least or up to about 18 hours, at least or up to about 19 hours, at least or up to about 20 hours, at least or up to about 21 hours, at least or up to about 22 hours, at least or up to about 23 hours, at least or up to about 24 hours, at least or up to about 2 days, at least or up to about 3 days at least or up to about 4 days at least or up to about 5 days at least or up to about 6 days, or at least or up to about 7 days.

The outcome in the cell can comprise regulation of a target gene. The regulation of the target gene can comprise a plurality of distinct modulations of the target gene. The plurality of gate units can each induce one of the plurality of distinct modulations of the target gene, such that a collection of the distinct modulation in concert yields a final expression and/or activity profile of the target gene. At least two distinct modulations of the plurality of distinct modulations can both increase an expression and/or activity level of the target gene. At least two distinct modulations of the plurality of distinct modulations can both decrease an expression and/or activity level of the target gene. Alternatively, a first distinct modulation of the plurality of distinct modulations can increase an expression and/or activity level of the target gene, while a second distinct modulation of the plurality of distinct modulations can decrease the expression and/or activity level of the target gene. In such case, the first distinct modulation can occur prior to the second distinct modulation, or vice versa. Alternatively, a distinct modulation (e.g., a first and/or second modulation) of the plurality of distinct modulations can maintain an expression and/or activity level of the target gene at the level of expression and/or activity level prior to the modulation.

In some cases, each distinct modulation of the plurality of distinct modulations of the target gene, as disclosed herein, can be necessary but individually insufficient to effect the desired expression and/or activity profile of the target gene. Thus, the outcome in the cell (e.g., enhanced cell function, induced cell state, etc.) induced by the plurality of distinct modulations of the target gene may not be possible in absence of any one of the plurality of distinct modulations of the target gene. Alternatively, a degree or measure of the outcome in the cell induced by the plurality of distinct modulations of the target gene can be greater than a degree or measure of the outcome in a control cell that is induced by none, one or more, but not all of the plurality of distinct modulations of the target gene, and/or by all of the plurality of distinct modulation of the target genes occurring through a different sequential order of events.

A second gate unit can be activated by a first gate unit (e.g. directly or indirectly). For example, the second gate unit can be directly activated by the first gate unit. Alternatively, the second gate unit can be activated by one or more additional gate units that are activated by the first gate unit (e.g., directly or indirectly). The one or more additional gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). The one or more additional gate units at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). Yet in another alternative, the second gate unit can be activated via another moiety responsible for activating the first gate unit (e.g., an activating moiety, a different gate unit, etc.).

The second gate unit can be activatable to induce inactivation of the first gate unit that has been activated. The terms “inactivation” or “disruption” may be used interchangeably herein. Inactivation and as disclosed herein can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

Inactivation by a gate moiety and/or a gene regulating moiety of the first gate unit as disclosed herein can be achieved through a endonuclease-based system (e.g., a CRISPR/Cas system). Alternatively or in addition to, inactivation can be achieved through the use of a transcriptional modulator system (e.g. a transcriptional repressor). An endonuclease-transcriptional modulator system (e.g., a Cas-repressor) can be used to achieve polynucleotide cleavage (e.g. for inactivating the gate moiety and/or the gene regulating moiety). Polynucleotide cleavage can create a nucleic acid modification such as a single-strand break, a double-strand break, an insertion, a deletion, or an insertion-deletion (indel). Alternatively or in addition to, the endonuclease-transcriptional modulator system (e.g., a Cas-repressor) can be used to modulate target gene expression.

Alternatively, the second gate unit can be activatable to amplify or enhance activation of the first gate unit that has been activated. Amplification or enhancement of the first gate unit can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

In some cases, a first gate unit modulates a first target gene. Alternatively, or in addition to, a first gate unit can also modulate a second gate unit. The modulation of the second gate unit can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or after the modulation of the first gate unit, as ascertained by rt-qPCR, Western blotting, or other methods.

In some cases, the second gate unit can modulate a second target gene. The modulation of the second target gene can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or more after the modulation of the first target gene, as ascertained by rt-qPCR, Western blotting, or other methods.

In some cases, modification of a target gene by a gate unit can inactivate a gene. For example, modification of a gene can stop expression and/or activity level of a target gene. Alternatively, modification of a gene can decrease the expression and/or activity level of a target gene. In some cases, modification of a gene can increase the expression and/or activity level of a target gene. Alternatively, modification of a gene can maintain the expression and/or activity level of a target gene.

An expression and/or activity profile of a gene of interest (e.g. a differentiation marker) can be compared to a control gene (e.g., a house keeping gene such as GAPDH), relative expression levels of two or more genes of interest (e.g., a ratio of expression or activity level between a stem cell marker and a differentiation marker), relative average expression levels of a gene of interest compared to average expression levels of that same gene of interest in a cell type of interest, etc.

In some cases, activation of the plurality of gate units may be a result of a single activation (e.g., by a single activating moiety at a single time point) of the heterologous genetic circuit. The plurality of gate units can comprise one of the first gate unit and the second gate that are preconfigured to be activated sequentially upon activation of the heterologous genetic circuit by the single activation. In some cases, one of the first and second gate unit can be activated by the single activating moiety (e.g., a guide nucleic acid), while the other of the first and second gate unit can be activated by an additional activating moiety (e.g., a different guide nucleic acid) that is different from the activating moiety of the heterologous genetic circuit. The additional activating moiety can be a part of the heterologous genetic circuit that is generated (e.g., expressed) only upon activation of the heterologous genetic circuit. Alternatively or in addition to, the first and second gate unit can each be activated by different activating moieties that are not the same as the activating moiety of the heterologous genetic circuit. Such different activating moieties can be parts of the heterologous genetic circuit that are generated (e.g., expressed) only upon activation of the heterologous genetic circuit.

In some embodiments of any one of the systems disclosed herein, a gate unit can comprise a gate moiety (e.g., at least or up to about 1 gate moiety, at least or up to about 2 gate moieties, at least or up to about 3 gate moieties, at least or up to about 4 gate moieties, at least or up to about 5 gate moieties, etc.) and/or a gene regulating moiety (e.g., at least or up to about 1 gene regulating moiety, at least or up to about 2 gene regulating moieties, at least or up to about 3 gene regulating moieties, at least or up to about 4 gene regulating moieties, at least or up to about 5 gene regulating moieties, at least or up to about 6 gene regulating moieties, at least or up to about 7 gene regulating moieties, at least or up to about 8 gene regulating moieties, at least or up to about 9 gene regulating moieties, at least or up to about 10 gene regulating moieties, etc.). A gate moiety as disclosed herein can comprise a guide nucleic acid molecule (gNA) (e.g., at least or up to about 1 gNA molecule, at least or up to about 2 gNA molecules, at least or up to about 3 gNA molecules, at least or up to about 4 gNA molecules, at least or up to about 5 gNA molecules, etc.). A gene regulating moiety as disclosed herein can comprise a gNA (e.g., at least or up to about 1 gNA molecule, at least or up to about 2 gNA molecules, at least or up to about 3 gNA molecules, at least or up to about 4 gNA molecules, at least or up to about 5 gNA molecules, etc.). The guide nucleic acid molecule as disclosed herein can comprise, but is not limited to, DNA, RNA, any analog of such, or any combination thereof. In some embodiments of any one of the systems disclosed herein, the gate moiety and/or the gene regulating moiety can be activatable to form a complex with an enzyme (e.g., an endonuclease and/or an exonuclease), and the complex can be configured to or capable of binding a target polynucleotide, e.g., to regulate expression and/or activity level of the target polynucleotide or another polynucleotide sequence operatively coupled to the target polynucleotide. For example, the complex can regulate expression and/or activity level of a gene comprising the target polynucleotide.

In some embodiments of any one of the systems disclosed herein, an initial (or the first) gate unit of the heterologous genetic circuit as disclosed herein may be activated (e.g., directly activated) by an activating moiety. The activating moiety can directly bind at least the portion of the initial gate unit to activate the initial gate unit, e.g., thereby to sequentially activate the heterologous genetic circuit. Alternatively, the activating moiety (e.g., electromagnetic energy) may activate the initial gate unit without directly binding the at least the portion of the initial gate unit. In some cases, the initial gate unit can comprise at least one gate moiety and at least one gene regulating moiety. In some cases, the initial gate unit can comprise at least one gate moiety but may not and need not comprise a gene regulating moiety. In some cases, the initial gate unit can comprise at least one gene regulating moiety but may not and need not comprise a gate moiety (e.g., the activating moiety may be configured to activate the initiate gate unit and at least one additional gate unit).

In some embodiments of any one of the systems disclosed herein, the gNA of the gate moiety and/or the gene regulating moiety (e.g., a gNA encoded by the gate moiety and/or the gene regulating moiety) can be an activatable gNA. The activatable gNA can be one of, but not limited to, any of the following: ribonucleotides (e.g., gRNA), deoxyribonucleotides, any analog of such, or any combination thereof. In some embodiments, a vector (or expression cassette) encoding the activatable gNA can comprise an inactivation polynucleotide sequence to render the gNA inactive until activated (e.g., until the inactivation polynucleotide sequence is modified or removed from the vector. For example, the inactivation polynucleotide sequence can encode a self-cleaving polynucleotide molecule (e.g., a ribozyme). Alternatively or in addition to, the inactivation polynucleotide sequence can encode non-canonical transcription termination sequence, as described below. The inactivation polynucleotide sequence can be a part of or adjacent to a region of the vector that encodes (i) a spacer sequence of the gNA, (ii) a scaffold sequence of the gNA, and/or (ii) any linker sequence between the spacer sequence and the scaffold sequence. The vector can comprise at least or up to about 1 inactivation polynucleotide sequence, at least or up to about 2 inactivation polynucleotide sequences, at least or up to about 3 inactivation polynucleotide sequences, at least or up to about 4 inactivation polynucleotide sequences, at least or up to about 5 inactivation polynucleotide sequences, at least or up to about 6 inactivation polynucleotide sequences, at least or up to about 7 inactivation polynucleotide sequences, at least or up to about 8 inactivation polynucleotide sequences, at least or up to about 9 inactivation polynucleotide sequences, or at least or up to about 10 inactivation polynucleotide sequences.

In some embodiments, the activatable gNA molecule can be a self-cleaving gNA (e.g., the gRNA contains a cis ribozyme). For example, when the activatable gNA is expressed in a cell, the activatable gNA may be self-cleavable to become non-functional (e.g., not configured to bind a target gene), unless a gene encoding the activatable gNA is modified prior to the expression of the activatable gNA. In some embodiments, the gNA can be synthetic. In some embodiments, the gNA can have a fluorescent label attached.

In some embodiments, the guide nucleic acid molecule encoded by the polynucleotide sequence as disclosed herein may comprise an enzymatic polynucleotide domain (e.g., a ribozyme). Alternatively, the guide nucleic acid molecule encoded by the polynucleotide sequence as disclosed herein may be capable of exhibiting an enzymatic activity by itself.

In some embodiments, the guide nucleic acid molecule encoded by the polynucleotide sequence as disclosed herein may not comprise an enzymatic polynucleotide domain (e.g., a ribozyme). Alternatively, the guide nucleic acid molecule encoded by the polynucleotide sequence as disclosed herein may not be capable of exhibiting an enzymatic activity by itself.

In some cases, the term “proGuide” as used herein may generally refer to such polynucleotide sequence (e.g., a vector, an expression cassette, a plasmid, etc.) that encodes the activatable gNA. The proGuide can be an example of a gate moiety. The proGuide can be an example of a gene regulating moiety. In some cases, the term “matureGuide” as used herein may generally refer to a functional form of the gNA that is expressed (e.g., transcribed) from the proGuide once the inactivation polynucleotide sequence (e.g., comprising a polyT sequence) is modified is removed from the proGuide.

In some cases, the heterologous genetic circuit can be activated by a guide nucleic acid molecule (gNA) (e.g., a functional gNA). Alternatively or in addition to, a gNA may be used to exhibit specific affinity to a target gene, to regulate the expression or the activity of the target gene. In some cases a gNA can be at least about 10, at least about 12, at least about 14, at least about 15, at least about 20, at least about 25, at least about 30, at least about 35, at least about 40, at least about 45, at least about 50, at least about 55, at least about 60, at least about 70, at least about 80, at least about 90, at least about 100, at least about 200, at least about 300, at least about 400, or at least about 500 bases in length. In some cases, a gNA can be at most about 500, at most about 400, at most about 300, at most about 200, at most about 150, at most about 100, at most about 90, at most about 80, at most about 70, at most about 60, at most about 55, at most about 50, at most about 45, at most about 40, at most about 35, at most about 30, at most about 25, at most about 20, at most about 15, at most about 14, at most about 12, or at most about 10 bases in length. In some cases, a gNA can be at least about 14 nucleotides in length. In some cases, a gNA can be at most about 300 nucleotides in length. In some cases, a gNA can be introduced to the system exogenously. Alternatively, a gNA can be produced endogenously by the system (e.g., be expressed by a gate unit).

A gNA can be activatable. A gNA can comprise a domain that corresponds to a tetraloop region of the guide nucleic acid molecule. A tetraloop can comprise four-base hairpin loop motif in RNA secondary structure that can cap a double-stranded section of nucleic acids. Tetraloops can play an important role in the structural stability and biological function of RNA. A tetraloop can also comprise the first hairpin in a gRNA.

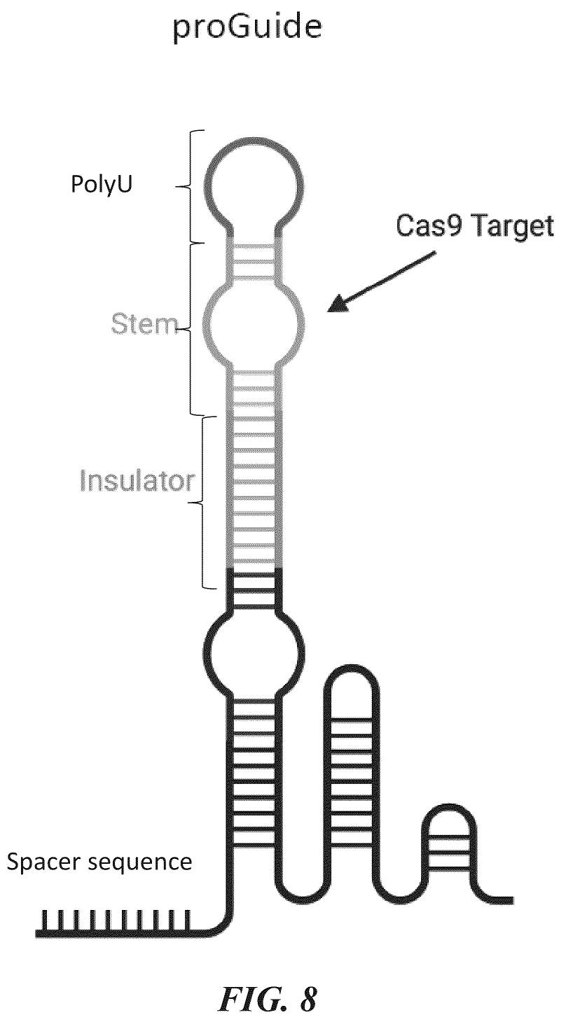

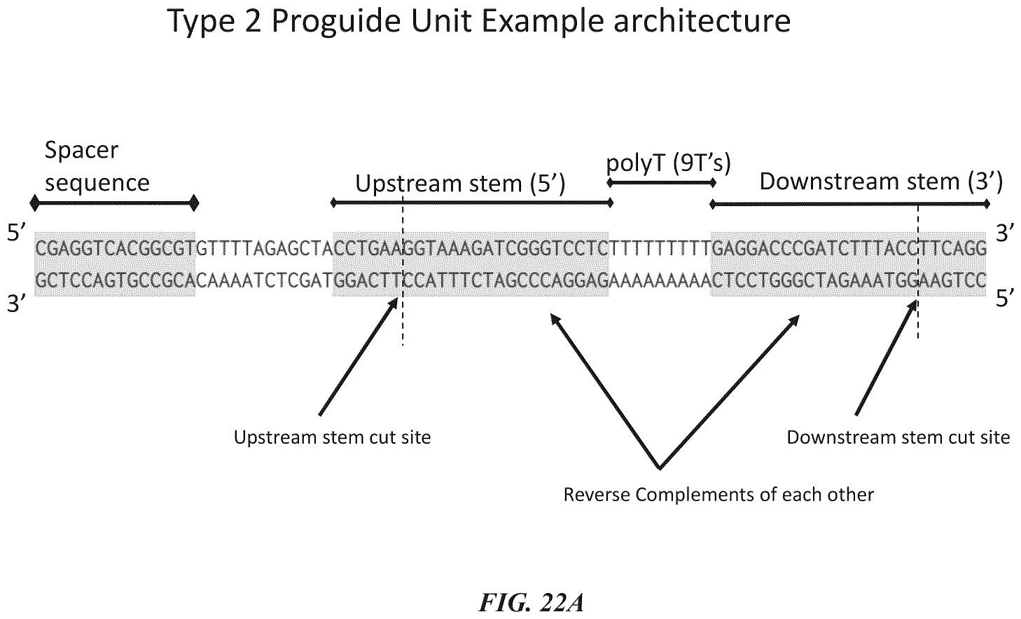

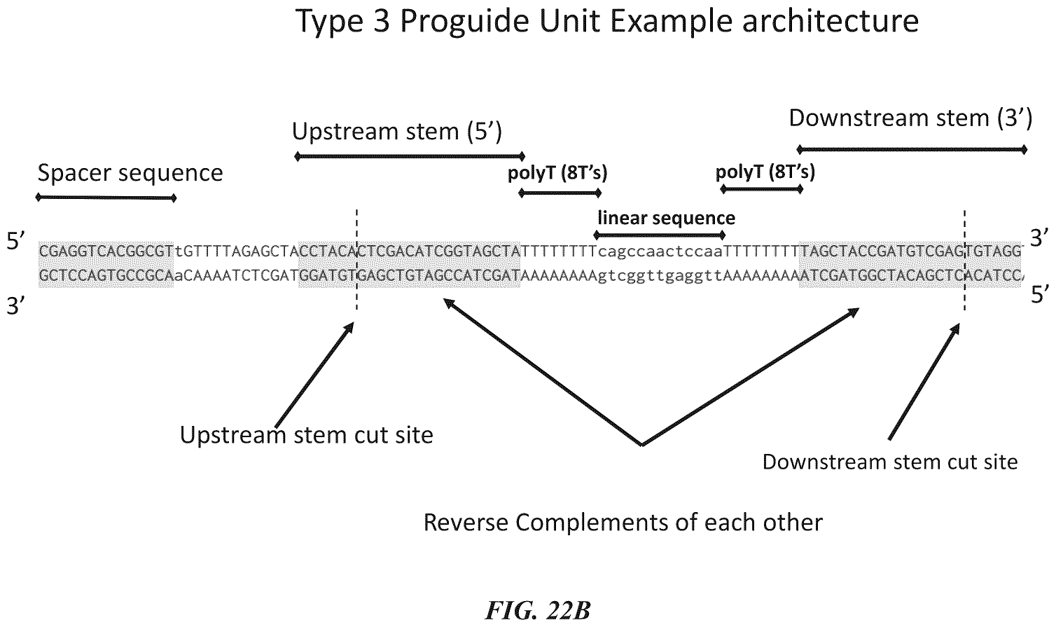

In some embodiments, a proGuide as provided herein can encode an activatable guide nucleic acid molecule, e.g., having the inactivation polynucleotide sequence (e.g., one or more poly X sequences, such as one or more polyT sequences). In some cases, a portion of the proGuide encoding the activatable guide nucleic acid molecule can comprise various regions that are sequentially linked (e.g., from 5′ to 3′), comprising upstream stem (e.g., an upstream cut site), a poly T unit (or “proUnit” as used interchangeably herein), and a downstream stem (e.g., a downstream cut site), as shown in TABLE 1 and TABLE 2. The upstream stem and the downstream stem may correspond to the “stem region” polynucleotide sequences that are at least partially complementary to each other, as schematically illustrated in the shape of the encoded guide nucleic acid molecule structure in FIG. 8. In some cases, the portion of the proGuide encoding the activatable guide nucleic acid molecule can comprise various regions that are sequentially linked (e.g., from 5′ to 3′), comprising the spacer sequence, an extra sequence (e.g., a linker sequence, an insulator sequence, or a sequence corresponding to a different portion of the scaffold sequence of the guide nucleic acid molecule), an upstream stem, a poly T unit, and a downstream stem. These various regions can be sequentially linked, e.g., from 5′ to 3′, in the order as illustrated in FIGS. 22A and 22B.

In some cases, the upstream and/or the downstream region may be or may comprise endonuclease recognition site as provided herein (e.g., that is targetable by Cas/guide nucleic acid complex), to modify or remove the polyT unit.

In some cases, upon modification or removal of the polyT unit, the guide nucleic acid molecule can be expressed, and at least a portion of the upstream stem and at least a portion of the downstream stem can form a part of a scaffold sequence of a functional guide nucleic acid molecule. Alternatively or in addition to, the at least the portion of the upstream stem and the at least the portion of the downstream stem may be coupled to the scaffold sequence of the functional guide nucleic acid molecule that does not hinder activity of the scaffold sequence to form a complex with a corresponding endonuclease (e.g., Cas protein, dCas protein, etc.), but may not be an actual or active part of the scaffold sequence). Thus, the upstream stem and/or the downstream stem can be characterized by (1) having sufficient length to be specifically targetable by a targeting moiety (e.g., a CRISPR/Cas/gRNA complex) for cleavage of the adjacent polyT sequence, (2) exhibiting minimal or substantially no sequence identity to any other polynucleotide sequence of a comparable length in the genome of the cell, to minimize or reduce off-target modification (e.g., cleavage) or endogenous genes, and/or (3) not having a secondary structure that can hinder the scaffold sequence's ability to form a complex with the corresponding endonuclease. Based at least on (2), the term “poly X”, “polyT”, “polyU”, “polyT unit”, “inactivation polynucleotide sequence,” “non-canonical sequence”, “non-canonical termination sequence” and “non-canonical disruption sequence” may be used interchangeably throughout the present disclosure.

A set of proGuides in a common heterologous genetic circuit can have identical (or substantially the same) or different extra sequences disposed between the spacer sequence and the upstream stem.

In some cases, in the proGuide, the distance between (i) the end (e.g., 3′ end) of a region that encodes or corresponds to the spacer sequence of a guide nucleic acid molecule and (ii) the end (e.g., 5′ end) of an additional region that corresponds to the inactivation polynucleotide sequence (e.g., polyT sequence) can be at least or up to about 5 nucleobases, at least or up to about 10 nucleobases, at least or up to about 11 nucleobases, at least or up to about 12 nucleobases, at least or up to about 13 nucleobases, at least or up to about 14 nucleobases, at least or up to about 15 nucleobases, at least or up to about 16 nucleobases, at least or up to about 17 nucleobases, at least or up to about 18 nucleobases, at least or up to about 19 nucleobases, at least or up to about 20 nucleobases, at least or up to about 21 nucleobases, at least or up to about 22 nucleobases, at least or up to about 23 nucleobases, at least or up to about 24 nucleobases, at least or up to about 25 nucleobases, at least or up to about 26 nucleobases, at least or up to about 27 nucleobases, at least or up to about 28 nucleobases, at least or up to about 29 nucleobases, at least or up to about 30 nucleobases, at least or up to about 31 nucleobases, at least or up to about 32 nucleobases, at least or up to about 33 nucleobases, at least or up to about 34 nucleobases, at least or up to about 35 nucleobases, at least or up to about 36 nucleobases, at least or up to about 37 nucleobases, at least or up to about 38 nucleobases, at least or up to about 39 nucleobases, at least or up to about 40 nucleobases, at least or up to about 41 nucleobases, at least or up to about 42 nucleobases, at least or up to about 43 nucleobases, at least or up to about 44 nucleobases, at least or up to about 45 nucleobases, at least or up to about 46 nucleobases, at least or up to about 47 nucleobases, at least or up to about 48 nucleobases, at least or up to about 49 nucleobases, at least or up to about 50 nucleobases, at least or up to about 51 nucleobases, at least or up to about 52 nucleobases, at least or up to about 53 nucleobases, at least or up to about 54 nucleobases, at least or up to about 55 nucleobases, at least or up to about 56 nucleobases, at least or up to about 57 nucleobases, at least or up to about 58 nucleobases, at least or up to about 59 nucleobases, at least or up to about 60 nucleobases, at least or up to about 65 nucleobases, at least or up to about 70 nucleobases, 75 nucleobases, at least or up to about 80 nucleobases, at least or up to about 85 nucleobases, at least or up to about 90 nucleobases, at least or up to about 95 nucleobases, or at least or up to about 100 nucleobases.

In some cases, at least one edit can be made to the polyX sequence. There can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15 or more edits made to a polyX sequence. There can be at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 edits made to a polyX sequence. An edit to a polyX sequence can be an insertion. Alternatively or in addition to, an edit to a poly X sequence can be a deletion. Alternatively, or in addition to, an edit to a polyX sequence can be an excision of the polyX sequence. Excision of the poly X sequence can be accomplished using two cut sites which flank the polyX sequence. An edit to a polyX sequence can utilize various forms of nucleic acid repair mechanisms such as, but not limited to, homology directed repair (HDR), non-homologous end joining (NHEJ) repair, and microhomology-mediated end joining (MMEJ) repair.

In some cases, at least one edit can be made to the polyT sequence. There can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15 or more edits made to a polyT sequence. There can be at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 edits made to a polyT sequence. An edit to a polyT sequence can be an insertion. Alternatively or in addition to, an edit to a polyT sequence can be a deletion. Alternatively, or in addition to, an edit to a polyT sequence can be an excision of the polyT sequence. Excision of the polyT sequence can be accomplished using two cut sites which flank the polyT sequence. An edit to a polyT sequence can utilize various forms of nucleic acid repair mechanisms such as, but not limited to, homology directed repair (HDR), non-homologous end joining (NHEJ) repair, and microhomology-mediated end joining (MMEJ) repair.

An edit to a polyX sequence in a gNA (e.g., a sgRNA) can affect expression of the guide nucleic acid molecule from the polynucleotide sequence. An edit to a polyX sequence can enhance expression, reduce expression, or silence expression of the gNA molecule from the polynucleotide sequence.

In some cases, modification of a polyX sequence can decrease the expression and/or activity level of the guide nucleic acid molecule by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a polyX sequence can decrease in the expression and/or activity level of the guide nucleic acid molecule by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyX sequence can increase the expression and/or activity level of the guide nucleic acid molecule by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a polyX sequence can increase in the expression and/or activity level of the guide nucleic acid molecule by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyX sequence can decrease the expression and/or activity level of the guide nucleic acid molecule by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable guide nucleic acid. Modification of a polyX sequence can decrease the expression and/or activity level of the guide nucleic acid molecule by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable guide nucleic acid.

In some cases, modification of a polyX sequence can increase the expression and/or activity level of the guide nucleic acid molecule by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable guide nucleic acid. Modification of a polyX sequence can increase the expression and/or activity level of the guide nucleic acid molecule by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable guide nucleic acid.

An edit to a polyT sequence in a gNA can affect expression of the guide nucleic acid molecule from the polynucleotide sequence. An edit to a polyT sequence can enhance expression, reduce expression, or silence expression of the gNA molecule from the polynucleotide sequence.

In some cases, modification of a polyT sequence can decrease the expression and/or activity level of the guide nucleic acid molecule by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a polyT sequence can decrease in the expression and/or activity level of the guide nucleic acid molecule by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyT sequence can increase the expression and/or activity level of the guide nucleic acid molecule by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a polyT sequence can increase in the expression and/or activity level of the guide nucleic acid molecule by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyT sequence can decrease the expression and/or activity level of the guide nucleic acid molecule by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable guide nucleic acid. Modification of a polyT sequence can decrease the expression and/or activity level of the guide nucleic acid molecule by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable guide nucleic acid.

In some cases, modification of a polyT sequence can increase the expression and/or activity level of the guide nucleic acid molecule by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable guide nucleic acid. Modification of a polyT sequence can increase the expression and/or activity level of the guide nucleic acid molecule by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable guide nucleic acid.

An edit to a polyX sequence in a gNA (e.g., a sgRNA) can affect expression of the guide nucleic acid molecule from the polynucleotide sequence, thereby regulating expression or activity of the target gene. An edit to a polyX sequence can enhance expression, reduce expression, or silence expression of the target gene.

In some cases, modification of a polyX sequence can decrease the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a polyX sequence can decrease in the expression and/or activity level of the target gene by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyX sequence can increase the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a polyX sequence can increase in the expression and/or activity level of the target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyX sequence can decrease the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable gene. Modification of a polyX sequence can decrease the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable gene.

In some cases, modification of a polyX sequence can increase the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable gene. Modification of a polyX sequence can increase the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable gene.

An edit to a polyT sequence in a gNA (e.g., a sgRNA) can affect expression of the guide nucleic acid molecule from the polynucleotide sequence, thereby regulating expression or activity of the target gene. An edit to a polyT sequence can enhance expression, reduce expression, or silence expression of the target gene.

In some cases, modification of a polyT sequence can decrease the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a polyT sequence can decrease in the expression and/or activity level of the target gene by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyT sequence can increase the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a polyT sequence can increase in the expression and/or activity level of the target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polyT sequence can decrease the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable gene. Modification of a polyT sequence can decrease the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable gene.

In some cases, modification of a polyT sequence can increase the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable gene. Modification of a polyT sequence can increase the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable gene.

In some cases, the termination of Pol-III controlled transcription can occur at non-canonical sequences. A non-canonical sequence can be in the form UUAUUU (which can also be written as its DNA complement, e.g., TTATTT or T₂AT₃). A non-canonical sequence can be T₃AT₂, T₃CT₂, T₂CT₃, T₃GT₂, T₂GT₃, T₃AT, TAT₃, T₃CT, TCT₃, T₃GT, TGT₃, T₂AT₂, T₂CT₂, or T₂GT₂. In some cases, a disrupted non-canonical termination sequence can be in the form UUAAUUU.

In some cases, the non-canonical termination sequence can comprise or consist substantially of a polynucleotide sequence exhibiting at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to the polynucleotide sequence of one or more members selected from the group consisting of SEQ ID NOs: 1-16, 36, and 45, or a complementary sequence thereof.

In some cases, the polynucleotide sequence comprising the non-canonical termination sequence (or a complementary sequence thereof) can have the following structure (I):

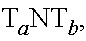

wherein: (i) “T” is a thymine nucleobase; (ii) “a” is an integer greater than or equal to 2; (iii) “b” is an integer greater than or equal to 2; and (iv) “N” is one or more nucleobases comprising at least one nucleobase is/are not T. The structure (I) as provided may be a consecutive sequence. The structure (I) may be a DNA sequence provided from 5′ to 3′.

In the structure (I), “a” and “b” may be the same number. Alternatively, “a” and “b” may not be the same number. For example, “a” may be greater than “b” by at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10. In another example, “b” may be greater than “a” by at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10.

In the structure (I), both of “a” and “b” can be at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 20.

In the structure (I), when Nis 1 or 2, N may not comprise (or may consist of) A, G, and/or C.

In the structure (I), when N is greater than or equal to 3, (i) the 5′ terminal nucleobase (e.g., that is directly adjacent to Tₐ) and the 3′ terminal nucleobase (e.g., that is directly adjacent to T_{b}) of N may not be T and (ii) one or more nucleobases disposed between the 5′ terminal nucleobase and the 3′ terminal nucleobase of N (e.g., “core region of N”) may be any nucleobase of the following: A, C, G, and/or T. In some cases, the core region of N may not comprise a consecutive polyT sequence (e.g., TT, TTT, TTTT, TTTTT, etc.). The core region of N may have a length of at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 30, at least or up to about 40, at least or up to about 50 nucleobases.

In some cases, the polynucleotide sequence comprising the non-canonical termination sequence (or a complementary sequence thereof) can have the following structure (II):

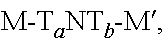

wherein: (i) TₐNT_{b} is as described above for the structure (I); (ii) M and M′ are polynucleotide sequences that are at least partially complementary to one another; and (iii) “-” is a polynucleotide linker or absent. In some cases, M and M′ can be targeted by the same gene editing moiety (e.g., Cas protein complexed with a guide RNA). For example, the structure (II) can be part of a double stranded vector, guide RNAs comprising the same spacer sequence can (1) generate a cut within M and generate an additional cut within the opposite/complementary strand of M′ or (2) generate a cut within the opposite/complementary strand of M and generate an additional cut at M′, thereby removing at least the 3′ portion of M (e.g., closer to Tₐ), substantially all of TₐNT_{b}, and at least the 5′ portion of M′ (e.g., closer to T_{b}), e.g., via one or more endogenous polynucleotide repair mechanisms such as MMEJ. In some cases, the number of removed nucleobases of M and the number of removed nucleobases of M′ can be the same or different. In some cases, the number of removed nucleobases of M and/or M′ can each be at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, or at least or up to about 30. As provided herein, the remaining (e.g., non-removed) portion of M and M′ can form a part of a scaffold sequence of a functional guide nucleic acid.

In some cases, the polynucleotide sequence comprising the non-canonical termination sequence (or a complementary sequence thereof) can have the following structure (II):

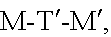

wherein: (i) T′ is the non-canonical termination sequence (e.g., polyT) as provided herein; and (ii) M and M′ are as described above for the structure (II).

In some cases, in the pair comprising M and M′ as shown in the structure (II) and/or the structure (III), the pair may form an insulator sequence, as provided herein. Alternatively, the pair may for a stem sequence, as provided herein.

In some cases, in the pair comprising M and M′ as shown in the structure (II) and/or the structure (III), a polynucleotide sequence of M and an additional polynucleotide sequence of M′ can, respectively, exhibit at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to the respective pair selected from the group consisting of (1) SEQ ID NO: 17 and SEQ ID NO: 54; (2) SEQ ID NO: 18 and SEQ ID NO: 55; (3) SEQ ID NO: 19 and SEQ ID NO: 56; (4) SEQ ID NO: 20 and SEQ ID NO: 57; (5) SEQ ID NO: 21 and SEQ ID NO: 58; (6) SEQ ID NO: 22 and SEQ ID NO: 59; (7) SEQ ID NO: 23 and SEQ ID NO: 60; (8) SEQ ID NO: 24 and SEQ ID NO: 61; (9) SEQ ID NO: 26 and SEQ ID NO: 62; (10) SEQ ID NO: 27 and SEQ ID NO: 63; (11) SEQ ID NO: 28 and SEQ ID NO: 64; (12) SEQ ID NO: 29 and SEQ ID NO: 65; (13) SEQ ID NO: 30 and SEQ ID NO: 66; (14) SEQ ID NO: 31 and SEQ ID NO: 67; (15) SEQ ID NO: 32 and SEQ ID NO: 68; (16) SEQ ID NO: 33 and SEQ ID NO: 69; (17) SEQ ID NO: 34 and SEQ ID NO: 70; and (18) SEQ ID NO: 35 and SEQ ID NO: 71, or complementary sequence pair thereof.

A non-canonical disruption sequence, also known as a non-canonical sequence or a non-canonical termination sequence, can cause premature termination. A non-canonical termination sequence can be modified by an endonuclease (e.g., a Cas9 endonuclease) to insert at least one nucleotide and thereby disrupt the non-canonical termination sequence. A non-canonical termination sequence can be altered by inserting at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10 nucleotides. Alternatively or in addition to, a non-canonical termination sequence can be modified by an endonuclease (e.g., a Cas9 endonuclease) to delete at least one nucleotide and thereby disrupt the non-canonical termination sequence. A non-canonical termination sequence can be altered by deleting at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 12, at least or up to about 13, at least or up to about 14, at least or up to about 15, at least or up to about 16, at least or up to about 17, at least or up to about 18, at least or up to about 19, at least or up to about 20, at least or up to about 25, at least or up to about 20, at least or up to about 25, at least or up to about 30, at least or up to about 35, at least or up to about 40, at least or up to about 45, at least or up to about 50, at least or up to about 55, at least or up to about 60, at least or up to about 65, at least or up to about 70, at least or up to about 75, at least or up to about 80, at least or up to about 90, or at least or up to about 100 nucleotides.

In some cases, a non-canonical termination sequence can be altered, thereby allowing expression of a functional variant of a guide nucleic acid molecule, by deleting at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% of the non-canonical termination sequence. For example, two ends of a desired portion of the non-canonical termination sequence (e.g., 5′ upstream stem and 3′ downstream stem that are disposed adjacent to the 5′ and 3′ ends of the polyT non-canonical termination sequence, as shown in FIGS. 22A and 22B, can be specifically targeted (e.g., via Cas/guide nucleic acid complex) to cut at or adjacent to the 5′ and 3′ ends of the polyT non-canonical termination sequence, to remove at least some or all of the polyT non-canonical termination sequence.

In some cases, the non-canonical termination sequence can be located within an RNA (e.g., not at a terminal end). In some cases, the non-canonical termination sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 3′ end of the polynucleotide sequence. In some cases, the non-canonical termination sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 5′ end of the polynucleotide sequence. In some cases, the non-canonical termination sequence can be located at a terminal end of a nucleic acid sequence.

In some cases, at least one edit can be made to the non-canonical termination sequence. There can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15 or more edits made to a polyX sequence. There can be at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 edits made to a non-canonical termination sequence. An edit to a non-canonical termination sequence can be an insertion. Alternatively or in addition to, an edit to a non-canonical termination sequence can be a deletion. Alternatively, or in addition to, an edit to a non-canonical termination sequence can be an excision of the non-canonical termination sequence. Excision of the non-canonical termination sequence can be accomplished using two cut sites which flank the non-canonical termination sequence. An edit to a non-canonical termination sequence can utilize various forms of nucleic acid repair mechanisms such as, but not limited to, homology directed repair (HDR), non-homologous end joining (NHEJ) repair, and microhomology-mediated end joining (MMEJ) repair.

In some cases, at least one edit can be made to the non-canonical termination sequence. There can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15 or more edits made to a non-canonical termination sequence. There can be at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 edits made to a non-canonical termination sequence. An edit to a non-canonical termination sequence can be an insertion. Alternatively or in addition to, an edit to a non-canonical termination sequence can be a deletion. An edit to a non-canonical termination sequence can utilize various forms of nucleic acid repair mechanisms such as, but not limited to, homology directed repair (HDR), non-homologous end joining (NHEJ) repair, and microhomology-mediated end joining (MMEJ) repair.

In some cases, modification of a non-canonical termination sequence can decrease the expression and/or activity level of the guide nucleic acid molecule by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a non-canonical termination sequence can decrease in the expression and/or activity level of the guide nucleic acid molecule by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a non-canonical termination sequence can increase the expression and/or activity level of the guide nucleic acid molecule by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a non-canonical termination sequence can increase in the expression and/or activity level of the guide nucleic acid molecule by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a non-canonical termination sequence can decrease the expression and/or activity level of the guide nucleic acid molecule by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable guide nucleic acid. Modification of a polyX sequence can decrease the expression and/or activity level of the guide nucleic acid molecule by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable guide nucleic acid.

In some cases, modification of a non-canonical termination sequence can increase the expression and/or activity level of the guide nucleic acid molecule by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a comparable guide nucleic acid. Modification of a non-canonical termination sequence can increase the expression and/or activity level of the guide nucleic acid molecule by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a comparable guide nucleic acid.

In some cases, an sgRNA comprises an additional termination sequence. An sgRNA can comprise at least about 1, at least about 2, at least about 3, at least about 4, at least about 5, or at least about 6 termination sequences.

In some cases, an sgRNA comprises a first termination sequence and a second termination sequence. In some cases the first termination sequence is a polyX sequence, and the second termination sequence is a polyX sequence. In some cases the first termination sequence is a polyX sequence, and the second termination sequence is a polyT sequence. In some cases the first termination sequence is a polyX sequence, and the second termination sequence is a non-canonical termination sequence. In some cases the first termination sequence is a polyT sequence, and the second termination sequence is a polyX sequence. In some cases the first termination sequence is a polyT sequence, and the second termination sequence is a polyT sequence. In some cases the first termination sequence is a polyT sequence, and the second termination sequence is a non-canonical termination sequence. In some cases the first termination sequence is a non-canonical termination sequence, and the second termination sequence is a polyX sequence. In some cases the first termination sequence is a non-canonical termination sequence, and the second termination sequence is a polyT sequence. In some cases the first termination sequence is a non-canonical termination sequence, and the second termination sequence is a non-canonical termination sequence.

In some cases, two termination sequences are adjacent to one another. Alternatively, or in addition to, two termination sequences can be separated by at least about 1, at least about 2, at least about 3, at least about 4, at least about 5, at least about 6, at least about 7, at least about 8, at least about, at least about 10, at least about 11, at least about 12, at least about 13, at least about 14, at least about 15, at least about 16, at least about 17, at least about 18, at least about 19, at least about 20, at least about 30, at least about 40, or at least about 50 nucleotides.

In some cases, an sgRNA comprises a first polyX sequence (e.g., a polyT sequence) and a second polyX sequence (e.g., a polyT sequence). In some cases the first poly X sequence and the second polyX sequence are the same. Alternatively, in some cases, the first poly X sequence and the second polyX sequence are different. In some cases a nucleobase length of the first poly X sequence and a nucleobase length the second polyX sequence are the same. Alternatively, in some cases, the nucleobase length of the first polyX sequence and the nucleobase length of the second poly X sequence are different. In some cases, the first polyX sequence and the second poly X sequence are separated by a non-polyX sequence (or non-termination sequence). In some cases the non-polyX sequence which is flanked by (e.g., disposed between) the first and second poly X sequences is at least about 1, at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 bases in length. In some cases, the non-polyX sequence which is flanked by (e.g., disposed between) the first and second polyX sequences is at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 bases in length.

In some cases, an sgRNA comprises a first polyT sequence and a second polyT sequence. In some cases the first polyT sequence and the second polyT sequence are the same. Alternatively, in some cases, the first polyT sequence and the second polyT sequence are different. In some cases, the first polyT sequence and the second polyT sequence are separated by a non-polyT sequence. In some cases the non-polyT sequence which is flanked by the polyT sequences is at least about 1, at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 bases in length. In some cases, the non-polyT sequence which is flanked by the polyT sequences is at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 bases in length.

In some cases, an sgRNA comprises a first non-canonical termination sequence and a second non-canonical termination sequence. In some cases the first non-canonical termination sequence and the second non-canonical termination sequence are the same. Alternatively, in some cases, the first non-canonical termination sequence and the second non-canonical termination sequence are different. In some cases, the first non-canonical termination sequence and the second non-canonical termination sequence are separated by a sequence that is not a non-canonical termination sequence (e.g., non-polyX sequence, such as non-polyT sequence). In some cases the sequence that is not a non-canonical termination sequence and which is flanked by the non-canonical termination sequences can be at least about 1, at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 bases in length. In some cases, the sequence that is not a non-canonical termination sequence and which is flanked by the non-canonical termination sequences is at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or at most about 1 bases in length.

When a guide nucleic acid molecule such as a guide RNA (or sgRNA) is described to comprise an element (e.g., one or more termination sequences, one or more polyX sequences, etc.), the description may refer to an expressed (e.g., transcribed) form of the guide nucleic acid molecule, or alternatively, may refer to a polynucleotide sequence that encodes such guide nucleic acid molecule, such as a vector or a plasmid. In some cases, when describing a polynucleotide sequence that encodes an activatable guide nucleic acid molecule (e.g., comprising polyT), such activatable guide nucleic acid molecule may be referred to as “guide nucleic acid molecule” or “guide RNA.”

In some cases, the polynucleotide sequence that encodes the guide nucleic acid molecule can comprise a domain comprising the polyT, which domain is disposed between two cut sites (e.g., upstream stem and downstream stem sites as provided herein) to permit removal of such domain for activation of the guide nucleic acid molecule. The domain can be a consecutive polynucleotide sequence. The domain can comprise the polyT sequence and a non-polyT sequence. The domain can have a length of at least or up to about 6 nucleobases, at least or up to about 8 nucleobases, at least or up to about 10 nucleobases, at least or up to about 12 nucleobases, at least or up to about 15 nucleobases, at least or up to about 20 nucleobases, at least or up to about 25 nucleobases, at least or up to about 30 nucleobases, at least or up to about 35 nucleobases, at least or up to about 40 nucleobases, at least or up to about 45 nucleobases, at least or up to about 50 nucleobases, at least or up to about 55 nucleobases, at least or up to about 60 nucleobases, at least or up to about 65 nucleobases, at least or up to about 70 nucleobases, at least or up to about 75 nucleobases, at least or up to about 80 nucleobases, at least or up to about 85 nucleobases, at least or up to about 90 nucleobases, at least or up to about 95, or at least or up to about 100 nucleobases. A proportion of the polyT sequence within the domain can be at least or up to about 510%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%. A proportion of the non-polyT sequence within the domain can be at least or up to about 510%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%.

In some cases, the polynucleotide sequence further comprises a region encoding an endonuclease recognition site. The endonuclease recognition site can be located adjacent to the region encoding the gNA molecule. The endonuclease recognition site can be located 5′ of the region encoding the gNA molecule. The endonuclease recognition site can be located 3′ of the region encoding the gNA molecule.

In some cases, the polynucleotide sequence can comprise a filler sequence that is adjacent to the region encoding the gNA molecule. In some cases, the polynucleotide sequence can comprise a filler sequence that is 5′ of the region encoding the gNA molecule. In some cases, the polynucleotide sequence can comprise a filler sequence that is 3′ of the region encoding the gNA molecule. In some cases, the polynucleotide sequence can comprise a region encoding a gNA molecule that is flanked by filler sequences. A filler sequence can be at least about 10, at least about 15, at least about 20, at least about 30, at least about 40, at least about 50, at least about 60, at least about 70, at least about 80, at least about 90, at least about 100, or more bases in length. A filler sequence can be at most about 100, at most about 90, at most about 80, at most about 70, at most about 60, at most about 50, at most about 40, at most about 30, at most about 20, at most about 15, at most about 10 or fewer bases in length.

In some cases, the polynucleotide sequence further comprises an insulator region. An insulator region can be an additional sequence which provides stability to a gNA molecule. The insulator region can be a sequence which comprises a sequence that is targetable by a gene editing moiety. For example, the insulator region can comprise a PAM sequence that is targetable by a Cas endonuclease.

The insulator region can comprise one PAM sequence. Alternatively, the insulator region can comprise more than one PAM sequence. An insulator region can have at least 1, at least 2, at least 3, at least 4, at least 5, at least 6, at least 7, at least 8, at least 9, or at least 10 PAM regions. An insulator region can have at most 10, at most 9, at most 8, at most 7, at most 6, at most 5, at most 4, at most 3, at most 2, or at most 1 PAM regions. An insulator region can have PAM sequences which face the same direction (e.g., PAM sequences that are in the 5′ to 3′ direction). Alternatively, an insulator region can have PAM sequence which face opposite directions (e.g., PAM sequences that are in both the 5′ to 3′ direction and the 3′ to 5′ direction).

The insulator region can be located between the transcriptional terminator region and the hairpin region of the gNA. The insulator region can be adjacent to the transcriptional terminator region (e.g., the polyU region). Alternatively, the insulator region can be non-adjacent to the transcriptional terminator region. The insulator region can be downstream of the transcriptional terminator region (e.g., the polyU region). The insulator region can be immediately downstream of the transcriptional terminator region (e.g., the polyU region). Alternatively, the insulator region can be upstream of the transcriptional terminator region (e.g., the polyU region). The insulator region can be immediately upstream of the transcriptional terminator region (e.g., the polyU region).

In some cases, the insulator region does not comprise a polyX region (e.g., a polyU region). Alternatively, the insulator region can comprise a polyX region. In some cases, the insulator region sequence is precisely defined. Alternatively, in some cases, the insulator region sequence is agnostic.

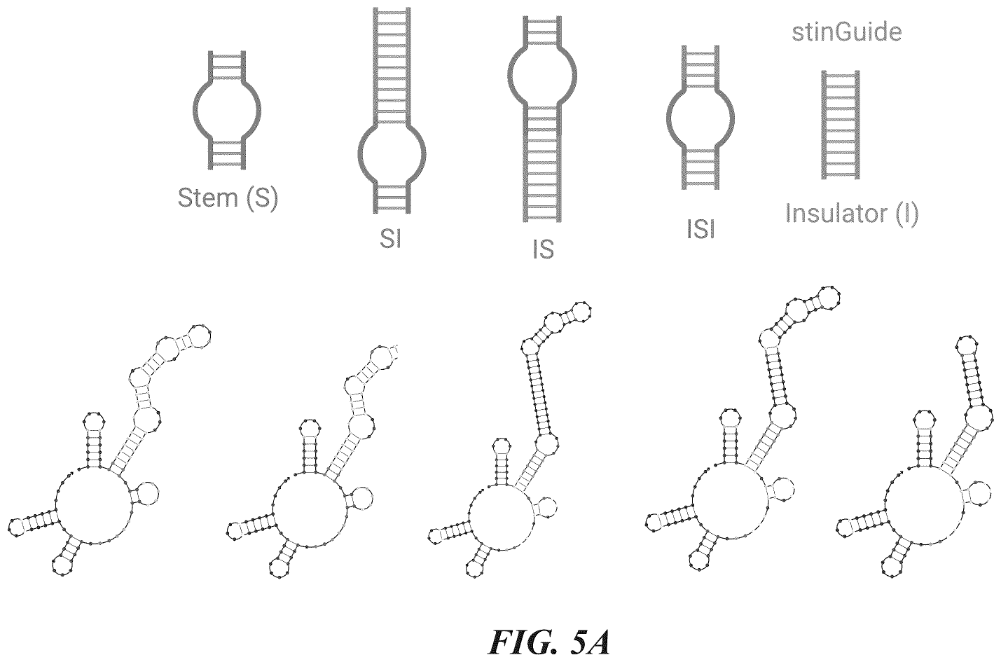

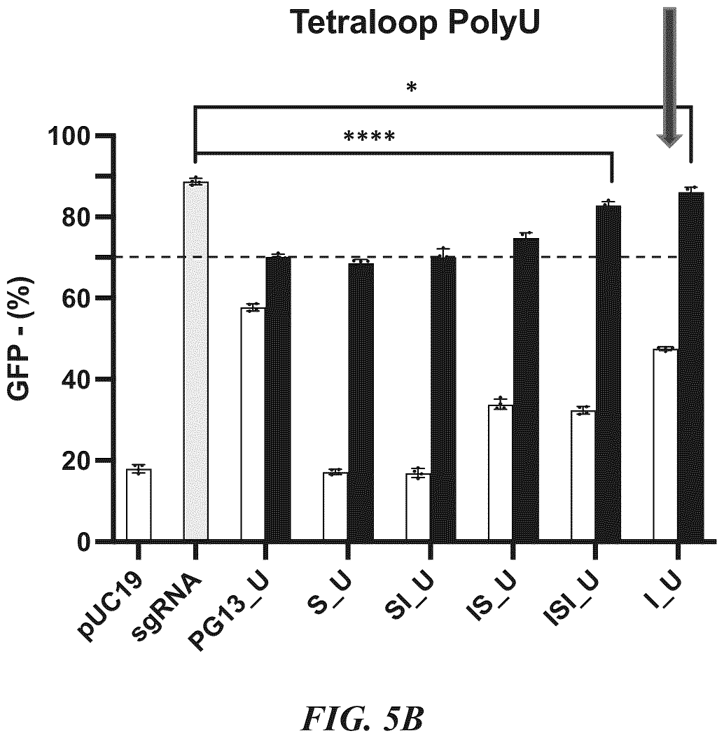

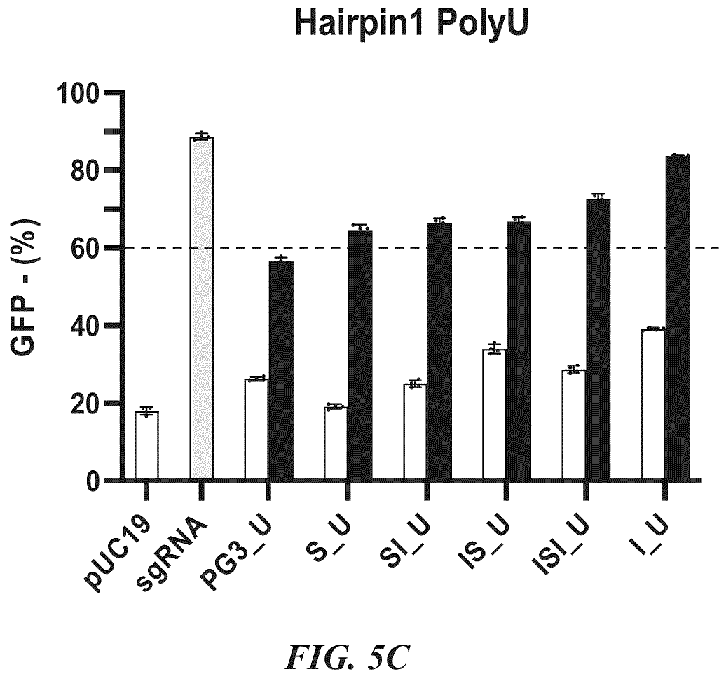

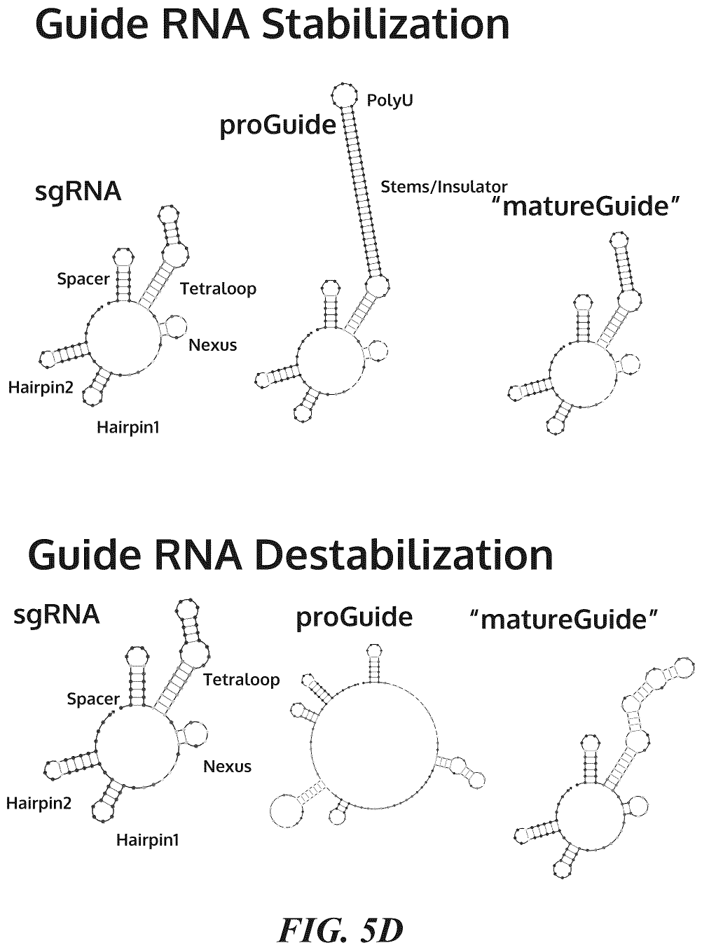

As seen in FIG. 5A, the insulator region can comprise a sequence that is fully complementary (I). Alternatively, or in addition to, the insulator region can comprise a sequence that comprises a stem(S), also described as a non-complementary bubble region. In some cases, the insulator region can comprise a sequence that comprises a non-complementary stem followed by a complementary region (SI). In some cases, the insulator region can comprise a sequence that comprises a complementary region followed by a non-complementary stem (IS). In some cases, the insulator region can comprise a sequence that comprises a non-complementary stem flanked by complementary regions (ISI).

In some cases, an insulator region can have multiple non-complementary stem regions. An insulator region can have at least 1, at least 2, at least 3, at least 4, at least 5, at least 6, at least 7, at least 8, at least 9, or at least 10 non-complementary stems. An insulator region can have at most 10, at most 9, at most 8, at most 7, at most 6, at most 5, at most 4, at most 3, at most 2, or at most 1 stems.

The additional sequence of the insulator region can be at least about 10, at least about 12, at least about 14, at least about 15, at least about 20, at least about 20, at least about 40, at least about 50, at least about 60, at least about 70, at least about 80, at least about 90, at least about 100, at least about 150, or at least about 200 nucleotides in length. The additional sequence of the insulator region can be at most about 200, at most about 150, at most about 100, at most about 90, at most about 80, at most about 70, at most about 60, at most about 50, at most about 40, at most about 30, at most about 20, or at most about 10 nucleotides in length.

In some cases, the addition of an insulator region can result in a gNA which has increased stability following modification by a gene editing moiety as compared to a gNA which lacked an insulator region. In some cases, the addition of a fully complementary insulator region can result in a in a gNA which has increased stability following modification by a gene editing moiety as compared to a gNA which comprises a stem region. Alternatively, the addition of one or more stem regions can result in a gNA which has increased stability following modification by a gene editing moiety as compared to a gNA which comprises a fully complementary insulator region.

In some cases, the addition of an insulator region can result in a gNA which has decreased stability following modification by a gene editing moiety as compared to a gNA which lacked an insulator region. In some cases, the addition of a fully complementary insulator region can result in a in a gNA which has decreased stability following modification by a gene editing moiety as compared to a gNA which comprises a stem region. Alternatively, the addition of one or more stem regions can result in a gNA which has decreased stability following modification by a gene editing moiety as compared to a gNA which comprises a fully complementary insulator region.

In some cases, the system of the present disclosure can further comprise an endonuclease capable of forming a complex with the gNA molecule. In some cases, the gNA-endonuclease complex can affect regulation of the expression or the activity of a target gene. An endonuclease can be a Type I endonuclease, a Type II endonuclease, or a Type III endonuclease. An endonuclease can be a Cas endonuclease (e.g., Cas9, Cas 10, Cas12, Cas13, Cas14, dCas).

In some cases, a guide nucleic acid molecules (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some cases, modification of a polynucleotide sequence (e.g., as a component of a gate unit, such as a gate moiety) or a target gene can be caused by a single-stranded break wherein there is a discontinuity in one nucleotide strand. Inactivation of a polynucleotide sequence or a target gene can be caused by at least about 1, at least about 2, at least about 3, at least about 4, at least about 5, at least about 6, at least about 7, at least about 8, at least about 9, at least about 10, or more single-stranded breaks. In some cases, inactivation of a gene can be caused by at most about 10, at most about 9, at most about 8, at most about 7, at most about 6, at most about 5, at most about 4, at most about 3, at most about 2, or at most about 1 single-stranded breaks.

In some cases, a gNA can have a size (e.g., including both spacer sequence and scaffold sequence) of at least or up to about 60 nucleotides, at least or up to about 70 nucleotides, at least or up to about 80 nucleotides, at least or up to about 85 nucleotides, at least or up to about 90 nucleotides, at least or up to about 95 nucleotides, at least or up to about 100 nucleotides, at least or up to about 105 nucleotides, at least or up to about 110 nucleotides, at least or up to about 120 nucleotides, at least or up to about 130 nucleotides, at least or up to about 140 nucleotides, at least or up to about 150 nucleotides, or at least or up to about 200 nucleotides.

In some cases, a scaffold sequence of a gNA can have a size of at least or up to about 30 nucleotides, at least or up to about 35 nucleotides, at least or up to about 40 nucleotides, at least or up to about 45 nucleotides, at least or up to about 50 nucleotides, at least or up to about 55 nucleotides, at least or up to about 60 nucleotides, at least or up to about 65 nucleotides, at least or up to about 70 nucleotides, at least or up to about 75 nucleotides, at least or up to about 80 nucleotides, at least or up to about 85 nucleotides, at least or up to about 90 nucleotides, at least or up to about 95 nucleotides, at least or up to about 100 nucleotides, at least or up to about 100 nucleotides, at least or up to about 120 nucleotides, at least or up to about 130 nucleotides, at least or up to about 140 nucleotides, or at least or up to about 150 nucleotides.

In some cases, a spacer sequence of a gNA can have a size of at least or up to about 10 nucleotides, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, or at least or up to about 30 nucleotides.

In some cases, the systems and methods of the present disclosure can utilize a single endonuclease system (e.g., a Cas-repressor) to achieve both (i) polynucleotide cleavage (e.g. for activating/inactivating the gate moiety and/or the gene regulating moiety) and (ii) modulation of target gene expression. When using a single endonuclease-transcriptional modulator system, unique guide nucleic acid molecules (gNAs) of differing spacer sequence lengths can be used to determine whether the single endonuclease-transcriptional modulator system may (i) hybridize to the polynucleotide sequence to induce Cas-mediated nuclease activity of the polynucleotide sequence, or (ii) can hybridize to a target gene (e.g., genomic DNA) to modulate expression and/or activity level of the target gene via action of the transcriptional activator without mediating Cas nuclease activity, as desired by the individual heterologous genetic circuit. For example, use of gNAs of differing spacer sequence lengths that bind to different targets can allow for a second gate unit as provided herein to induce inactivation of a first gate unit that has been activated and/or induce a distinct modulation of a second target gene.

As abovementioned, the length the spacer sequence of the gNA can affect the ability of the gNA to mediate Cas nuclease activity. In some cases, gNAs with spacer sequences of differing lengths can be used in the same heterologous genetic circuit to affect different types of cleavage, activation, inactivation, and/or modulation of one or more target nucleic acids. In some cases, a gNA spacer sequence that is shorter than a threshold length (e.g., about 16 nucleotides) can preclude nuclease activity of a Cas-transcriptional modulator, while still mediating DNA binding for transcriptional modulation of a target gene. In some cases, a gNA spacer sequence that is shorter than at least about 25 nucleotides, at least about 20 nucleotides, at least about 19 nucleotides, at least about 18 nucleotides, at least about 17 nucleotides, at least about 16 nucleotides, at least about 15 nucleotides, at least about 15 nucleotides, at least about 14 nucleotides, at least about 13 nucleotides, at least about 12 nucleotides, at least about 11 nucleotides, or at least about 10 nucleotides can preclude nuclease activity of a Cas protein while still mediating DNA binding.

For example, a gNA comprising a 20-nucleotide spacer sequence (e.g., a gNA encoded by a gate moiety for targeting a gene regulating moiety plasmid) can be sufficient to facilitate nuclease activity of an endonuclease (e.g. a Cas or a Cas-transcriptional modulator fusion protein) at a target polynucleotide sequence. Alternatively or in addition to, a gNA comprising a 14-nucleotide spacer sequence (e.g., a gNA encoded by a gene regulating moiety) can hybridize to DNA but may not be long enough to mediate nuclease activity—it can only facilitate endonuclease binding to the cognate DNA sequence. Accordingly, the shorter gNA can selectively allow for transcriptional modulation of a target gene though the use of a endonuclease-transcriptional modulator system (e.g. a Cas-activator system, a Cas-repressor system), without cleavage of the target gene.

In some cases, modification of a polynucleotide sequence (e.g., as a component of a gate unit, such as a gate moiety) or a target gene can be caused by a double-stranded break wherein there is a discontinuity in both nucleotide strands. In some cases, a number of such double-stranded break (e.g., necessary for such modification) can be at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10. In some cases, modification of a polynucleotide sequence (e.g., as a component of a gate unit, such as a gate moiety) or a target gene can be caused by an indel, also known as an insertion-deletion mutation. An indel mutation can comprise a frameshift or non-frameshift mutation. An indel mutation can comprise a point mutation, also called a base substitution, wherein only one base or base pair is modified. An indel mutation can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or more bases or base pairs in length. An indel mutation can comprise at most about 2000, at most about 1000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 bases or base pairs in length.

In some cases, modification of a polynucleotide sequence (e.g., as a component of a gate unit, such as a gate moiety) or a target gene can be achieved without cleavage of the polynucleotide sequence or the target gene. For example, a gene regulating moiety (e.g., a nucleic acid molecule and/or an endonuclease, such as a complex comprising a CRISPR/Cas protein and a guide nucleic acid molecule) can specifically bind to the polynucleotide sequence or the target gene, such that expression and/or activity of the polynucleotide sequence or the target gene is modified. The gene regulating moiety can comprise a transcriptional repressor or a transcriptional activator, as provided herein. Alternatively or in addition not, the gene regulating moiety can induce epigenetic modification (or epigenome modification) as provided herein.

In some cases, the modification of the polynucleotide sequence or the target gene, as provided herein, can inactivate the polynucleotide sequence or the target gene. For example, modification of the polynucleotide sequence or the target gene can repress or reduce expression and/or activity level of the polynucleotide sequence or the target gene. In some cases, the modification of the polynucleotide sequence or the target gene, as provided herein, can activate the polynucleotide sequence or the target gene. For example, modification of the polynucleotide sequence or the target gene can increase expression and/or activity level of the polynucleotide sequence or the target gene.

In some cases, the modification of the polynucleotide sequence or the target gene, as provided herein, can comprise decreasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 95%, at least or up to about 99%, or about 100% (e.g., as compared to a control that, for example, lacks the modification).

In some cases, the modification of the polynucleotide sequence or the target gene, as provided herein, can comprise decreasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, or at least or up to about 100-fold (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 150%, at least or up to about 200%, at least or up to about 300%, at least or up to about 400%, or at least or up to about 500% (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, at least or up to about 100-fold, at least or up to about 200-fold, at least or up to about 300-fold, at least or up to about 400-fold, at least or up to about 500-fold, or at least or up to about 1,000-fold (e.g., as compared to a control that, for example, lacks the modification).

In some cases, the control expression and/or activity level of the comparable guide nucleic acid, as disclosed herein, can refer to expression and/or activity level of the guide nucleic acid molecule from the same polynucleotide sequence, but without the modification of the polyX sequence, such as the polyT sequence within the polynucleotide sequence. In some cases, the control expression and/or activity level of the comparable guide nucleic acid, as disclosed herein, can refer to expression and/or activity level of a comparable guide nucleic acid molecule from a control polynucleotide sequence that encodes the comparable guide nucleic acid molecule, wherein a domain of the control polynucleotide sequence that corresponds to a tetraloop region of the comparable guide nucleic acid molecule does not comprise a polyX sequence (e.g., polyT sequence) as provided herein.

As provided herein, when the heterologous genetic circuit is activated to induce a plurality of distinct modulations of a target gene, as provided herein, the plurality of distinct modulations of the target gene can be different (e.g., different degrees of change in the expression and/or activity level of the target gene. For example, a first modulation exerted by a first gene unit and second modulation exerted by a second gate unit can be different by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500%. The first modulation and the second modulation can be different by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. Alternatively or in addition to, the distinct modulation of the target gene can be substantially the same (e.g., the same).

The plurality of distinct modulations can be individually sufficient to induce the desired change in expression and/or activity level of the target gene. Alternatively, the distinct modulations can be individually insufficient to induce the desired change in expression and/or activity level of the target gene.

One or more target genes as disclosed herein can comprise one or more endogenous genes (e.g., genomic DNA, mRNA, mitochondrial DNA, etc.), exogenous genes, transgenes, or a combination thereof.

One or more target genes as disclosed herein can comprise a cell differentiation regulatory factor, a molecular function regulatory factor, a binding factor, a fusogenic factor, a protein folding chaperone, a protein tag, a RNA folding chaperone, a cell signaling factor, an immune response factor, a sensory receptor, a cell structural factor, a protein binding factor, a cargo receptor, a catalytic factor, or a small molecule sensor.

In some cases, a target gene may be subjected to at least two distinct modulations comprising a first modulation and a second modulation. Timing of the first modulation and the second modulation can be controlled (e.g., as predetermined by the design of the heterologous genetic circuit). For example, the onset of the second modulation (e.g., by at least a portion of the second gate unit, such as the second gene regulation moiety) can occur subsequent to the onset of the first modulation (e.g., by at least a portion of the first gate unit, such as the first gene regulating moiety) by at least about 1 second, at least about 2 seconds, at least about 3 seconds, at least about 4 seconds, at least about 5 seconds, at least about 6 seconds, at least about 7 seconds, at least about 8 seconds, at least about 9 seconds, at least about 10 seconds, at least about 20 seconds, at least about 30 seconds, at least about 40 seconds, at least about 50 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 20 hours, at least about 1 day, at least about 2 days, at least about 3 days, at least about 4 days, at least about 5 days, at least about 6 days, at least about 7 days, at least about 8 days, at least about 9 days, or at least about 10 days. The onset of the second modulation (e.g., by at least a portion of the second gate unit, such as the second gene regulation moiety) can occur subsequent to the onset of the first modulation (e.g., by at least a portion of the first gate unit, such as the first gene regulation moiety) by at most about 10 days, at most about 9 days, at most about 8 days, at most about 7 days, at most about 6 days, at most about 5 days, at most about 4 days, at most about 3 days, at most about 2 days, at most about 1 day, at most about 20 hours, at most about 10 hours, at most about 9 hours, at most about 8 hours, at most about 7 hours, at most about 6 hours, at most about 5 hours, at most about 4 hours, at most about 3 hours, at most about 2 hours, at most about 1 hours, at most about 50 minutes, at most about 40 minutes, at most about 30 minutes, at most about 20 minutes, at most about 10 minutes, at most about 9 minutes, at most about 8 minutes, at most about 7 minutes, at most about 6 minutes, at most about 5 minutes, at most about 4 minutes, at most about 3 minutes, at most about 2 minutes, at most about 1 minutes, at most about 50 seconds, at most about 40 seconds, at most about 30 seconds, at most about 20 seconds, at most about 10 seconds, at most about 9 seconds, at most about 8 seconds, at most about 7 seconds, at most about 6 seconds, at most about 5 seconds, at most about 4 seconds, at most about 3 seconds, at most about 2 seconds, or at most about 1 second.

In some cases, a number of gate units that need to be activated (e.g., sequentially activated) between the activation of the first modulation by the first gate unit and the later activation of the second modulation by the second gate unit can at least in part determine (e.g., substantially determine) the timing between the first modulation and the second modulation. Upon activation of the first modulation of the target gene by the first gate unit, at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more additional gate units may need to be activated (e.g., sequentially activated) to activate the second gate unit for inducing the second modulation. Upon activation of the first modulation of the target gene by the first gate unit, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 additional gate units may need to be activated (e.g., sequentially activated) to activate the second gate unit for inducing the second modulation.

The outcome of a cell can comprise the regulation of a plurality of target genes. For example, the outcome can comprise the regulation of at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more target genes. The outcome can comprise the regulation of at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 target gene(s). Each gene that is disclosed herein can be subjected to at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more modulations. Each gene that is disclosed herein can be subjected to at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 modulation(s). One or more modulations of a target gene (e.g., an endogenous gene), as induced by the heterologous genetic circuit of the present disclosure, may be an artificial modulation (or a heterologous modulation) that may otherwise not occur in the cell in absence of (i) the heterologous genetic circuit and/or (ii) the activating moiety of the heterologous genetic circuit.

The plurality of gate units can operate sequentially (e.g., each of the plurality of gate units is activated in a sequential manner). For example, a gate unit of the plurality to be activated to activate a subsequent gate unit of the plurality. Sequential operation of the gate units can be linear. Alternatively, sequential operation of the gate units can route back on one another as inputs to form a loop. For example, a plurality of the gate units can induce a feedback loop such as a positive feedback loop or a negative feedback loop.

In some embodiments of any one of the systems disclosed herein, the first gate unit can comprise a first gene regulating moiety that can be activatable to exhibit specific binding to the target gene to induce a first distinct modulation. Alternatively or in addition to, the first gate unit can comprise a first gene regulating moiety that can be activatable to exhibit non-specific binding to the target gene to induce the first distinct modulation.

The first distinct modulation can induce a change (e.g., increase or decrease) in the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more, as compared to a control expression and/or activity level of a gene that is not targeted by the first distinct modulation. The first distinct modulation can induce a change (e.g., increase or decrease in the expression and/or activity level of the target gene by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less, as compared to a control expression and/or activity level of a gene that is not targeted by the first distinct modulation.

The first distinct modulation as disclosed herein (e.g., induced by the first gate unit) can induce a change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to as compared to a control expression and/or activity level of a gene that is not targeted by the first distinct modulation. The first distinct modulation can induce a change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a gene that is not targeted by the first distinct modulation.

In some cases, the control expression and/or activity level of the gene that is not targeted by the first distinct modulation, as disclosed herein, can refer to expression and/or activity level of a housekeeping gene (e.g., a constitutive gene that controls basal cellular function). In some cases the control expression and/or activity level of the gene that is not targeted by the first distinct modulation, as disclosed herein, can refer to expression and/or activity level of a gene that is controlled by a second distinct modulation. In some cases, the control expression and/or activity level of the gene that is not targeted by the first distinct modulation, as disclosed herein, can refer to expression and/or activity level of a gene that is controlled by a second genetic circuit. In some cases, the control expression and/or activity level of the gene that is not targeted by the first distinct modulation, as disclosed herein, can refer to expression and/or activity level of a gene that acts in the same metabolic pathway as the target gene. Alternatively, the control expression and/or activity level of the gene that is not targeted by the first distinct modulation, as disclosed herein, can refer to expression and/or activity level of a gene that does not act in the same metabolic pathway as the target gene.

Subsequently, a second distinct modulation as disclosed herein (e.g., induced by the second gate unit) can induce an additional change (e.g., increase, decrease, or selective attenuation) in the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%, as compared to a control expression and/or activity level of a gene that is not targeted by the second distinct modulation. The second distinct modulation can induce an additional change (e.g., increase or decrease) in the expression and/or activity level of the target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%, as compared to a control expression and/or activity level of a gene that is not targeted by the second distinct modulation.

The additional change via the second distinct modulation can induce an additional change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a gene that is not targeted by the second distinct modulation. The second distinct modulation can induce an additional change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a gene that is not targeted by the second distinct modulation.

The additional change via the second distinct modulation can occur when the expression and/or activity level of the target gene reaches a target level via action of the first distinct modulation, e.g., by design of the heterologous genetic circuit.

The additional change via the second distinct modulation can occur when the expression and/or activity level of the target gene is changed (e.g., increased or decreased) via action of the first distinct modulation by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level of a gene that is not targeted by the second distinct modulation. The additional change via the second distinct modulation can occur when the expression and/or activity level of the target gene is changed (e.g., increased or decreased) via action of the first distinct modulation by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level of a gene that is not targeted by the second distinct modulation.

Alternatively, or in addition to, a second distinct modulation as disclosed herein (e.g., induced by the second gate unit) can induce a change (e.g., increase or decrease) in the expression and/or activity level of an additional target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%, as compared to a control expression and/or activity level of a gene that is not targeted by the second distinct modulation. The second distinct modulation can induce a change (e.g., increase or decrease) in the expression and/or activity level of the additional target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%, as compared to a control expression and/or activity level of a gene that is not targeted by the second distinct modulation.

In some cases, the control expression and/or activity level of the gene that is not targeted by the second distinct modulation, as disclosed herein, can refer to expression and/or activity level of a housekeeping gene (e.g., a constitutive gene that controls basal cellular function). In some cases the control expression and/or activity level of the gene that is not targeted by the second distinct modulation, as disclosed herein, can refer to expression and/or activity level of a gene that is controlled by the first distinct modulation. In some cases the control expression and/or activity level of the gene that is not targeted by the second distinct modulation, as disclosed herein, can refer to expression and/or activity level of a gene that is controlled by a third distinct modulation. In some cases, the control expression and/or activity level of the gene that is not targeted by the second distinct modulation, as disclosed herein, can refer to expression and/or activity level of a gene that is controlled by a second genetic circuit. In some cases, the control expression and/or activity level of the gene that is not targeted by the second distinct modulation, as disclosed herein, can refer to expression and/or activity level of a gene that acts in the same metabolic pathway as the target gene. Alternatively, the control expression and/or activity level of the gene that is not targeted by the second distinct modulation, as disclosed herein, can refer to expression and/or activity level of a gene that does not act in the same metabolic pathway as the target gene.

A cell can comprise a prokaryotic cell, a eukaryotic cell, or an artificial cell. A cell can be a fungal cell, a plant cell or an animal cell (e.g., a mammalian cell). A cell (e.g., an initial cell to be modified into the engineered cell as disclosed herein, a final cell product generated from the engineered cell as disclosed herein, etc.) can comprise a muscle cell, an immune cell, a neuron, an osteoblast, an endothelial cell, an mesenchymal cell, an epithelial cell, a stem cell, an secretory cell, a blood cell, a germ cell, a nurse cell, a storage cell, an enteroendocrine cell, a pituitary cell, a neurosecretory cell, a duct cell, an odontoblast, a cementoblast, a glial cell, or an interstitial cell.

Non-limiting examples of such a cell can include lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

The present disclosure also provides a composition comprising the engineered genetic modulators and/or the engineered genetic circuits as disclosed herein. The composition can further comprise the actuator of the heterologous genetic circuit(s). The present disclosure also provides a kit comprising the composition. The kit can further comprise the activator(s) of the heterologous genetic circuit(s). The activator(s) can be in the same composition as the engineered genetic modulators and/or the engineered genetic circuits. Alternatively or in addition to, the activator(s) can be in a different and separate composition from the engineered genetic modulators and/or the engineered genetic circuits.

### EXAMPLES

### Example 1: Deactivating sgRNA Activity

In this example, an RNA polymerase III transcriptional termination sequence (polyT tract) is shown to be sufficient to deactivate sgRNA activity. Ribozymal activity is compared to polyU effectivity in deactivating sgRNAs.

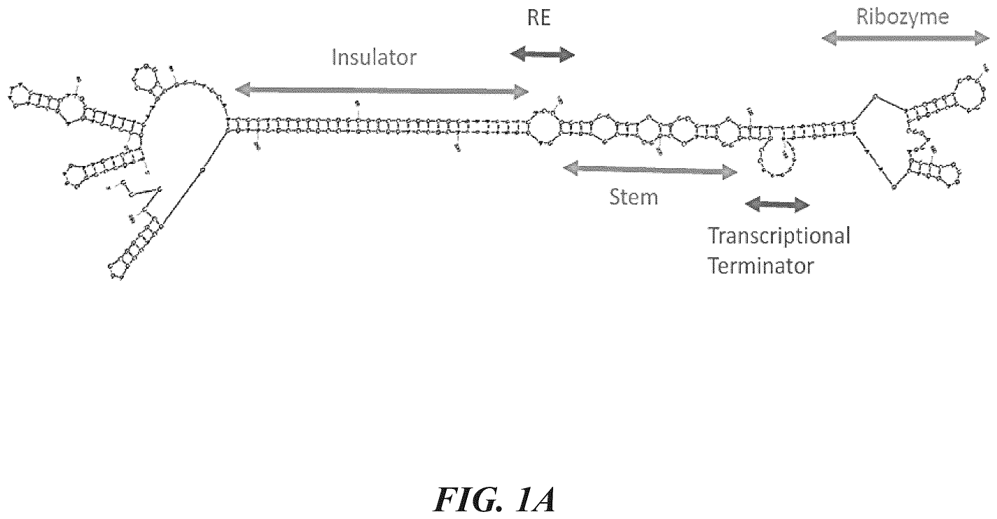

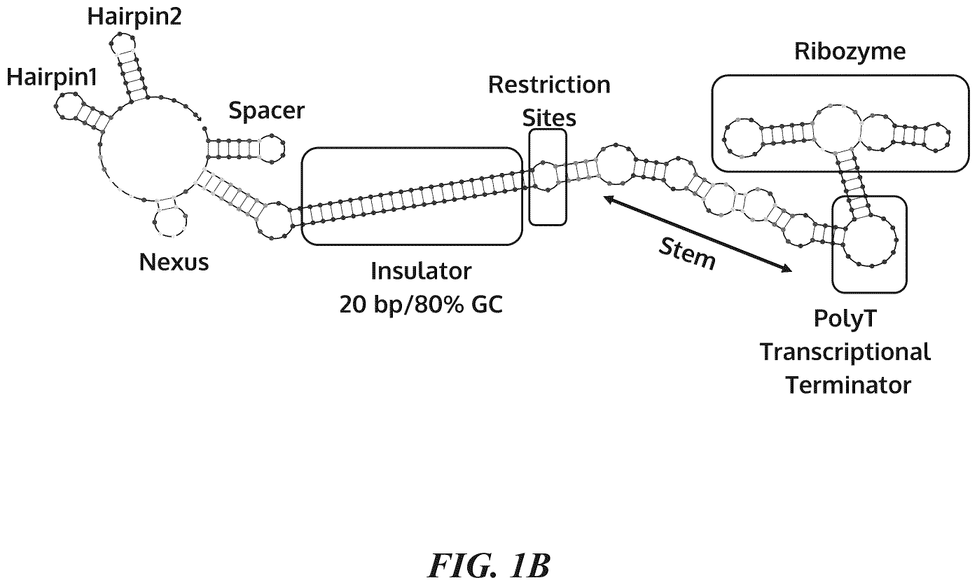

In vitro RNA analysis was performed to determine ribozyme catalytic capacity with modifications to various secondary structures. FIGS. 1A-1B show exemplary ribozymal sgRNA;

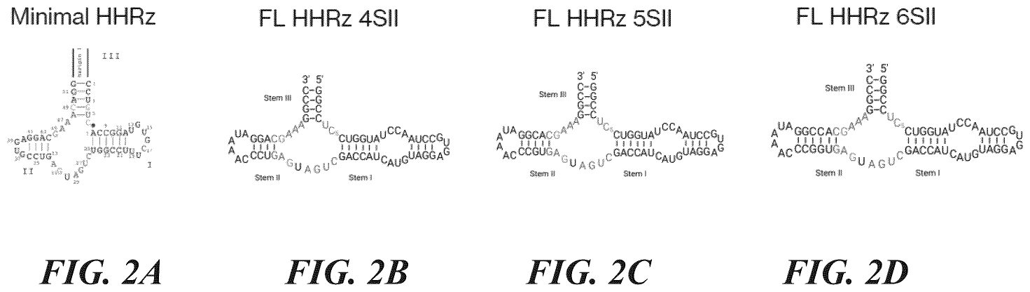

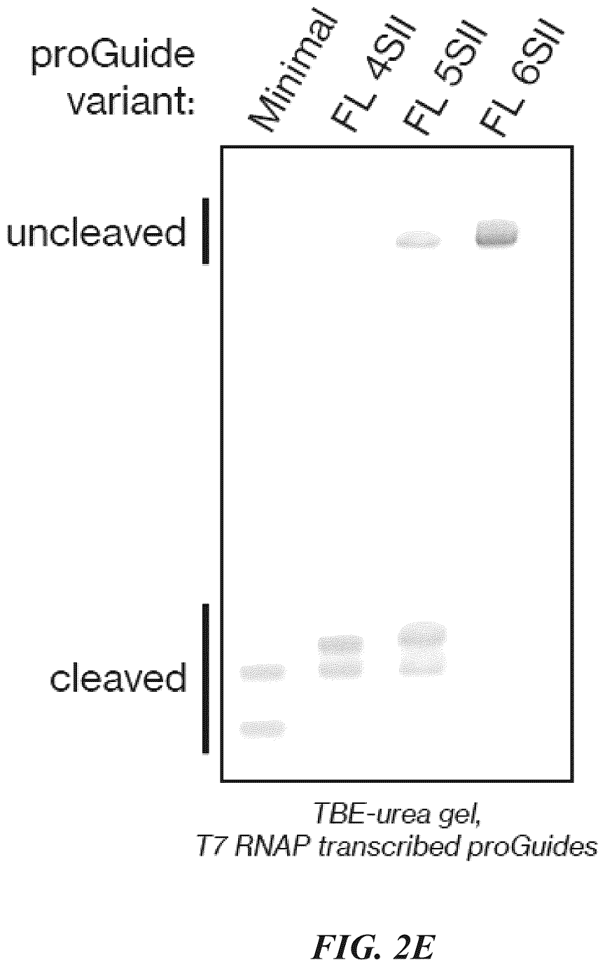

FIGS. 2A-2D show variations of secondary RNA structures. FIG. 2E shows that while certain alteration to stem I and stem III did not hinder ribozyme activity, elongation of stem II disrupted ribozyme activity.

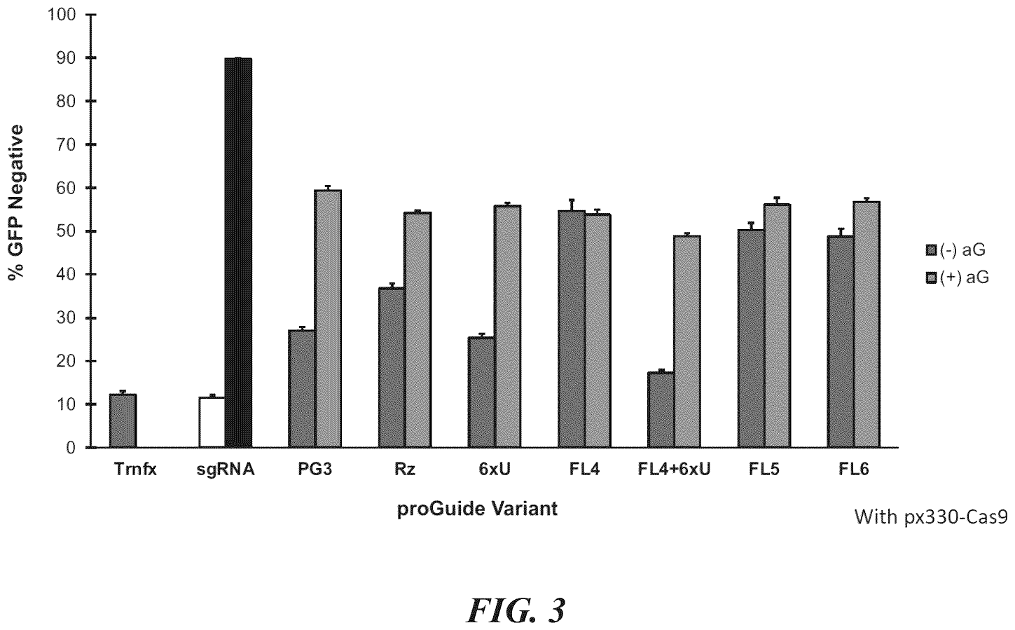

Next, various modifications were tested for their ability to inactivate guide nucleic acids (FIG. 3). PG3 is a gNA with a stem, a GFP spacer, and a hairpin with a modified ribozyme and 6U; Rz is a gNA with a modified ribozyme; 6×U is a gNA with a 6U polyU sequence; FL4 is a gNA with a full-length ribozyme; FL4+6×U is a gNA with a full-length ribozyme and a 6U polyU sequence; FL5 is a gNA with an extended full length ribozyme; FL6 is a different gNA with an extended full-length ribozyme. Both sgRNA which targeted GFP directly (sgRNA) and a transfection control in which cells received no Cas9 or sgRNA (Trnfx) were used as controls. Ag+ indicates samples that received the activating guide nucleic acid (gNA) while ag-indicates samples that did not receive the activating gNA.

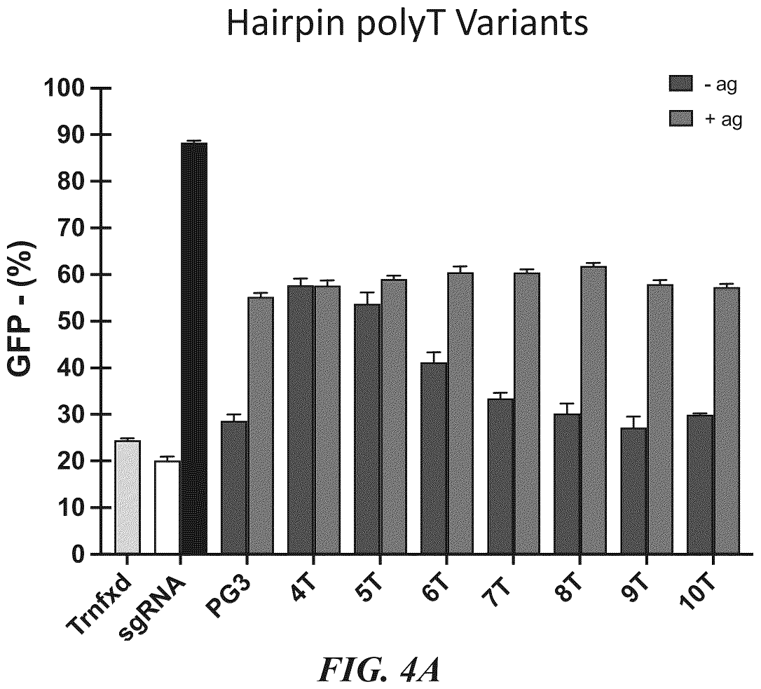

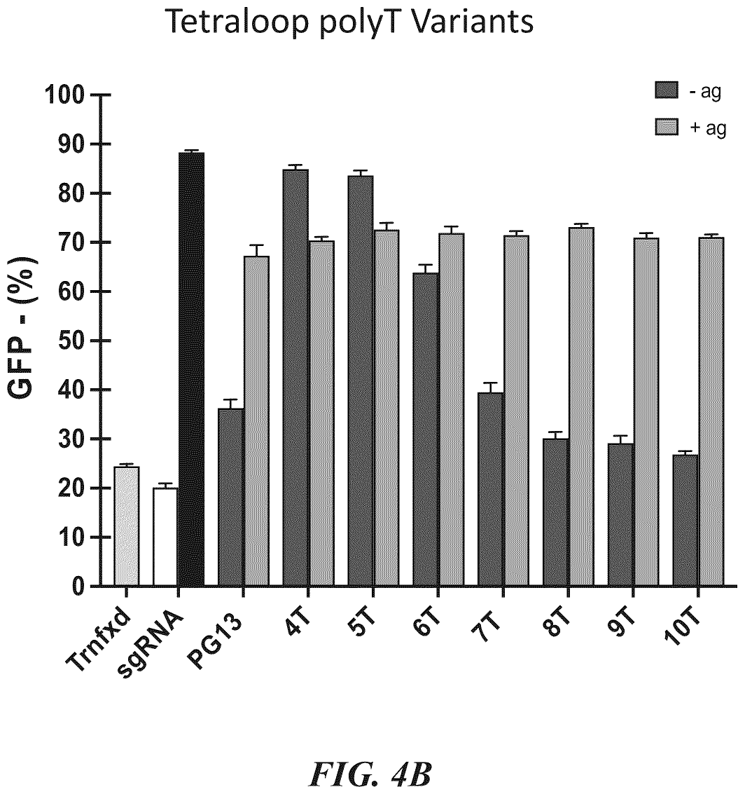

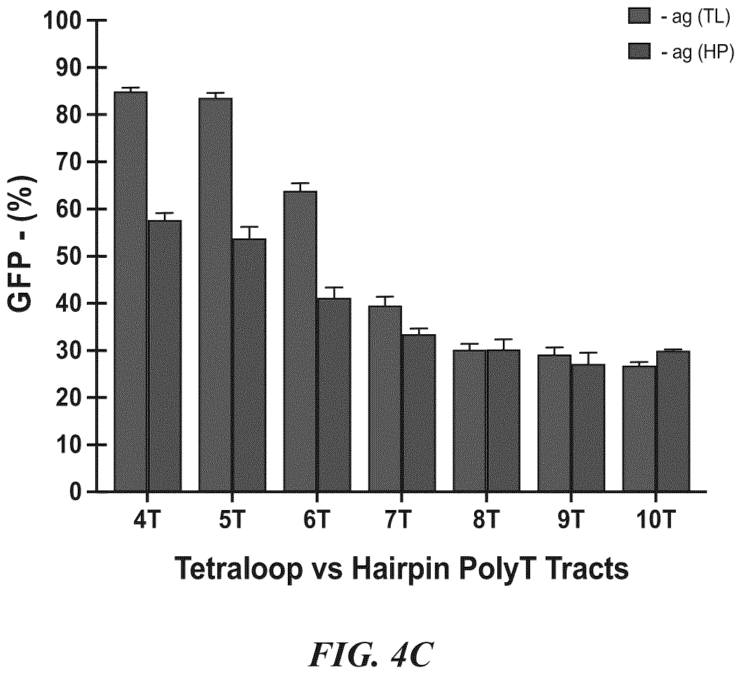

The polyU termination sequence was shown to be sufficient to inactivate the guide nucleic acid. PolyU sequences (polyT sequences in the DNA) with increasing length were sufficient to inactivate the gNA both when located in the hairpin (FIG. 4A) and when located in the tetraloop (FIG. 4B). Additionally, longer polyU sequences were increasingly efficient in their termination efficiency; capping at around 8T (FIG. 4C).

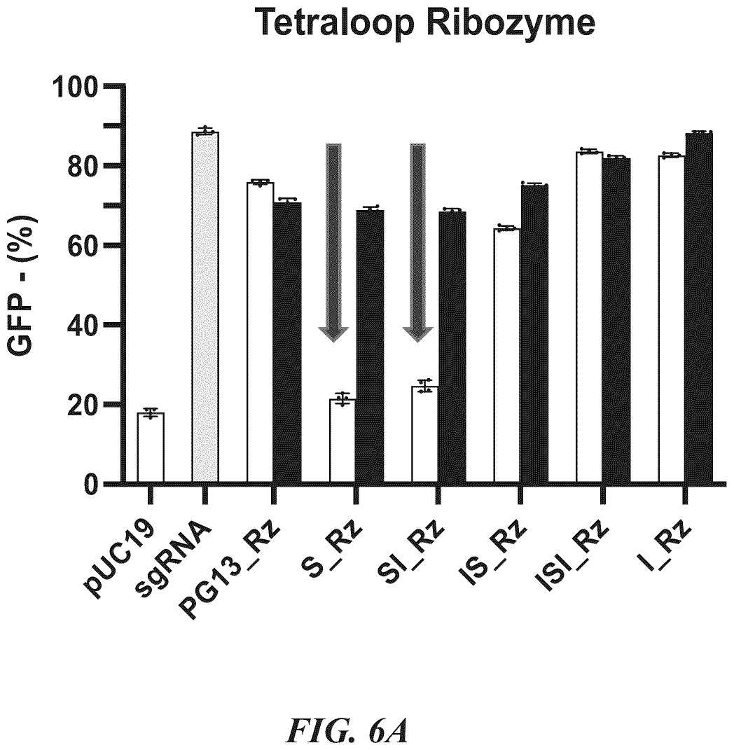

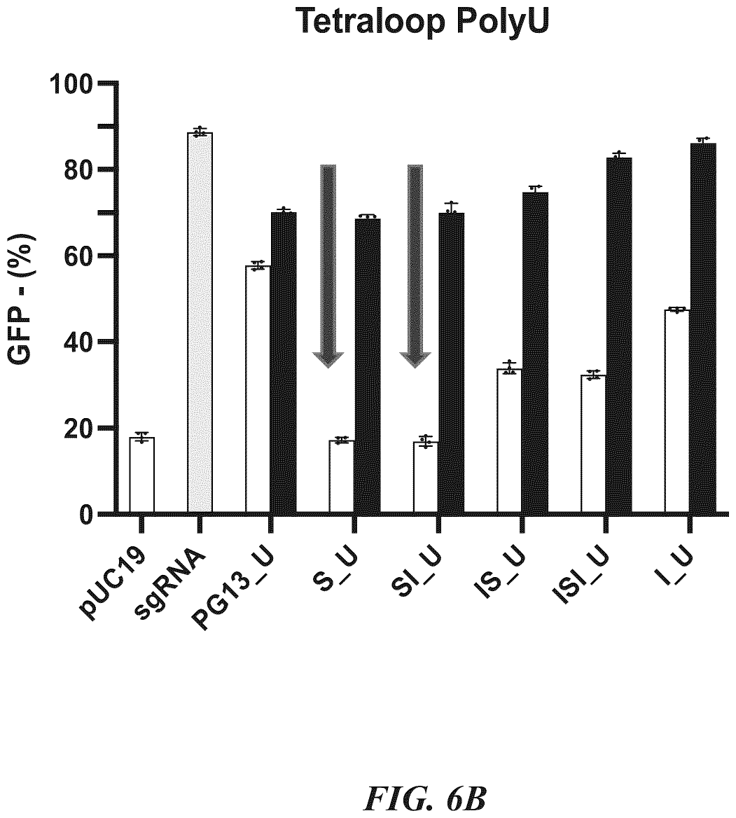

When an inactivation sequence is flanked on each side by insulator and/or stem regions, the orientation of those insulator/stem sequences within the DNA can be arranged such that the RNA can form secondary structures. When the same DNA sequence is placed in a direct repeat orientation at the two locations, then the RNA will form non-complementary bubble structures illustrated with the Stem(S). When the DNA sequence is placed in an inverted repeat orientation, then the RNA can form complementary structures illustrated with the Insulator (I). When the DNA sequence at the each site is a mixture of direct and inverted repeat orientation, it can form RNA structures comprised of complementary regions and non-complementary bubble structures at different locations illustrated in SI, IS, and ISI. These abbreviations, I, S, SI, IS, ISI are used in FIG. 5B,C and FIG. 6A,B.

The most significant conversion of an inactive proGuide to an active matureGuide occurred when the polyT tract was flanked by stem sequences oriented in the inverted repeat arrangement (I_U) either when the proUnit was placed in the hairpin 1 (FIG. 5B) or tetraloop (FIG. 5C) location within the gNA. The lowest level of activation occurred when the stem sequences were arranged in the direct repeat orientation (S_U) in hairpin 1 (FIG. 5B) and tetraloop (FIG. 5C) variants.

When comparing the inactivation efficiency of insulator regions when paired with a ribozyme rather than a polyU region, both the stem (S_Rz) or a stem followed by a complementary sequence (SI_Rz) preceding the ribozyme most enhanced inactivation when the ribozyme was located in the tetraloop (FIG. 6A) to a level comparable to polyU (FIG. 6B). However, the S and SI orientation enabled the weakest conversion efficiency to an active matureGuide (black bars), and the polyU was significantly more effective at inactivating the proGuide in ISI and I orientations.

These experiments showed that the polyT termination sequence is sufficient to act as the inactivation module of a sgRNA. Furthermore, secondary structure caused by the orientation of sequences flanking the polyT sequence can modulate its effect on termination efficiency, as can length of the polyT itself. Conversion to an active matureGuide RNA is also affected by the orientation of the sequences flanking the polyT.

### Example 2: Optimization of sgRNA Deactivation

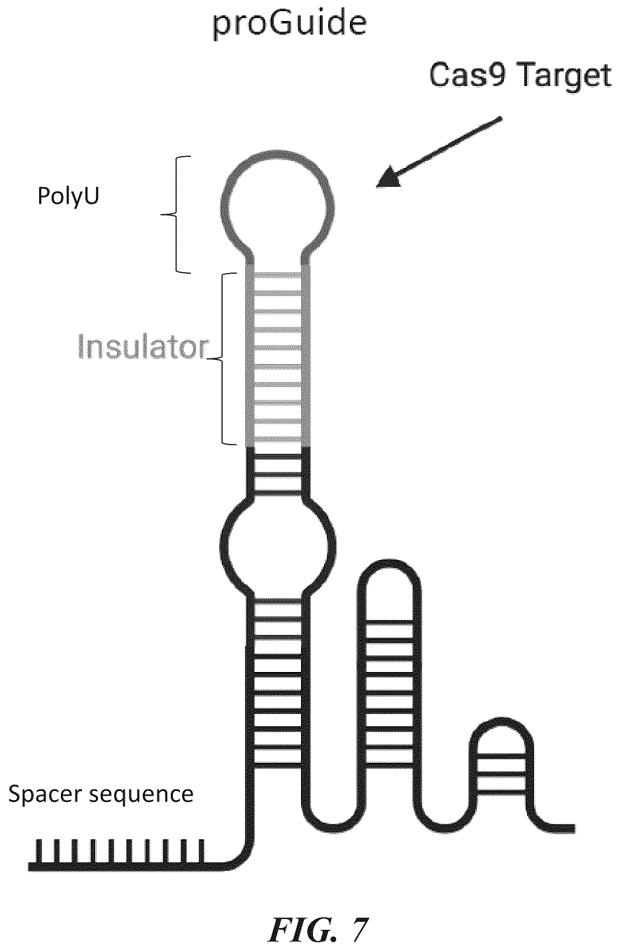

In this prophetic example, the effect of the sequences flanking the polyT tract is examined in the case of possible readthrough transcription by RNA Pol III to synthesize a complete guide RNA from proGuide DNA templates. In the Insulator (I) arrangement with a single polyT tract, a readthrough transcription event would generate a proGuide with an extension of the tetraloop and extension of hairpin (FIG. 7). This extension can be predicted to form a stable guide RNA that could function with Cas (e.g. Cas9) or a variant thereof. With the insulator-stem (IS) orientation, readthrough transcription would generate a proGuide with a longer extension on the end of the tetraloop, and the longer extension would have more complex secondary structure (FIG. 8). The more complex secondary structure can be predicted to interfere with Cas (e.g. Cas9) activity or a variant thereof and reduce residual activity of the proGuide before it is converted to an active state by removal of the stems and polyT tract. However, in some cases, presence of a polyT track that sufficiently terminates readthrough (e.g., transcription) of the complete guide RNA may be more efficient at reducing (or preventing) the change of forming a complex with the Cas protein, thereby being more efficient at interfering with the Cas protein's activity and reducing residual activity.”

### Example 3: Conversion of an Inactive proGuide to an Active matureGuide

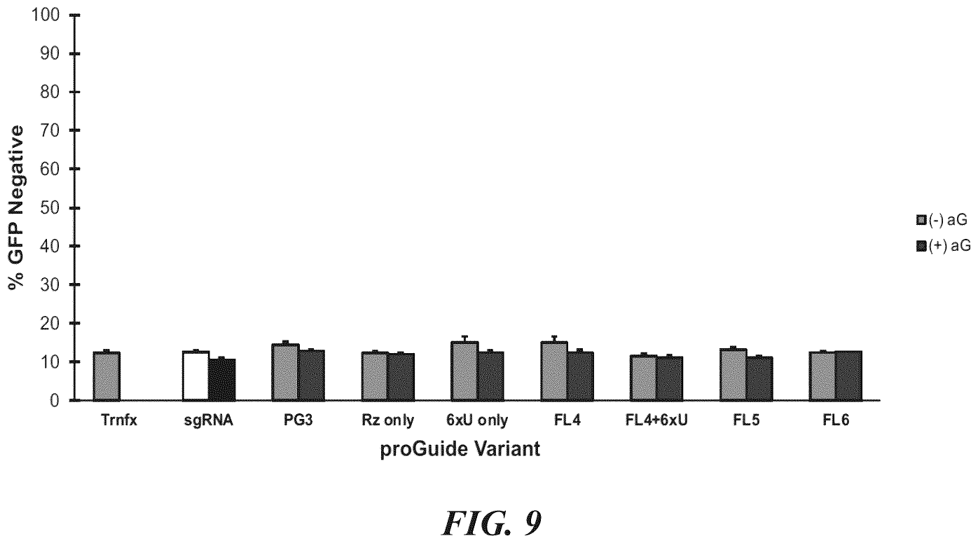

Systems and methods provided herein disclose the conversion of a nucleic acid molecule from an inactive state to an active state. In some embodiments, the nucleic acid molecule is a proGuide, which can be converted from an inactive state to an active state. In this example, genetic circuits utilized sgRNAs or variant modifications thereof to disrupt GFP output requiring Cas9 endonuclease activity, as shown by lack of GFP disruption when a enzymatically inactive dCas9 is used (FIG. 9). The importance of the GFP disruption data is that they show conversion of an inactive proGuide with a spacer targeting GFP to an active matureGuide state that mutates a genomic transgene (e.g. EGFP). The conversion occurs by Cas9 activity at the proGuide cut sites by the activating Guide sgRNA (aGuide).

### Results

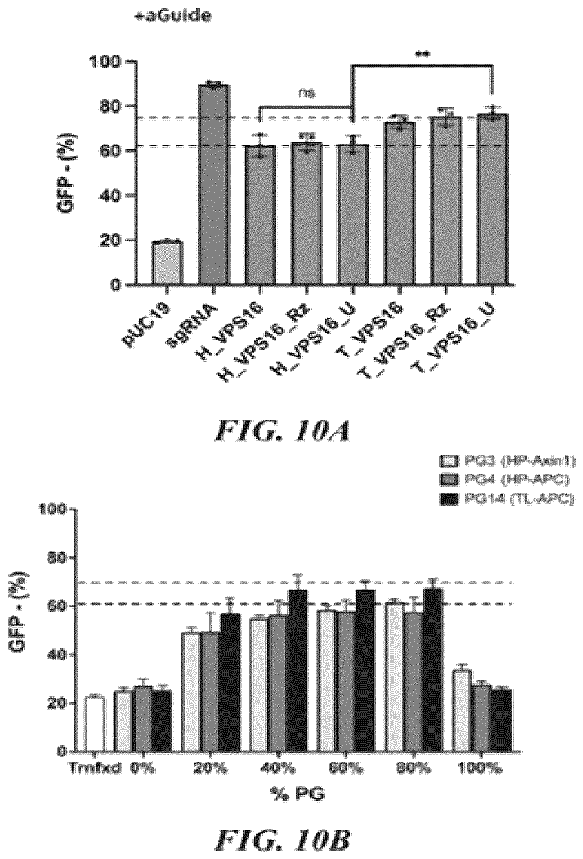

Conversion of proGuides using a polyT tract for inactivation was examined with several proGuide variants possessing the same spacer targeting GFP but with different inactivation moieties. FIG. 10A shows the activity of proGuides converted to matureGuides by an aGuide for variants with insertion of a ribozyme (Rz) or a polyT tract (U), or both in either the hairpin 1 (H) or tetraloop (T) site. Note that the cut sites (e.g. VPS16) for each of the variants are the same and are in the same orientation. This experiment shows that the proGuides with different inactivation sequences but identical cut site sequences and orientations displayed the same activity as matureGuides. MatureGuides derived from some insertions (e.g. tetraloop insertions) displayed higher activity than those derived from other insertions (e.g. hairpin 1 insertions). This experiment also showed that each of these matureGuides was less active in cells (fewer GFP-negative cells) than the sgRNA control that targeted GFP.

FIG. 10B shows that changing the concentration of proGuide relative to aGuide in transfection mixes had relatively minor effects on the frequency of GFP disruption in cells. In this experiment, 0% proGuide (PG) indicates level of GFP negative cells with transfection of the aGuide and no proGuide. 100% is level of GFP negative cells with transfection of proGuide with no aGuide. The higher level of activity from the proGuide with some insertions (e.g. tetraloop insertion) over that of proGuides with other insertions (e.g. hairpin insertion) indicates a cap on activity is not caused by levels of the guide RNA in cells.

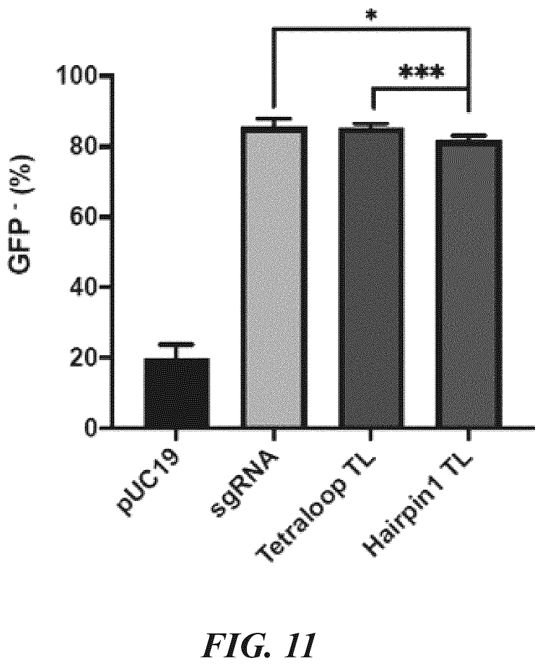

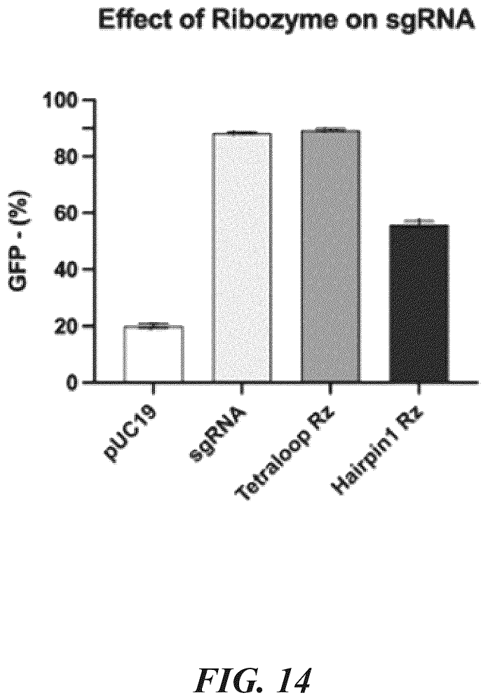

There is minimal effect of insulator sequences without a proUnit inactivation sequence on sgRNA activity (FIG. 11). It was also shown that when a ribozyme is inserted without stems or insulator sequences, and thus without potential disruptive structural effects of the inserted sequences, the ribozyme activity was not sufficient to significantly inactivate the sgRNA (FIG. 14).

### Example 4: Non-Canonical RNA Pol III Terminators

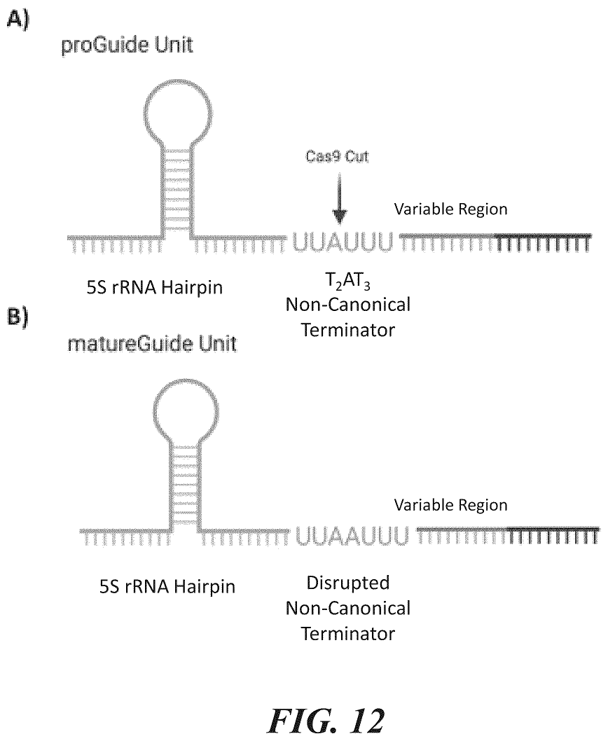

In this prophetic example, non-canonical terminator sequences, such as those shown in FIG. 12, are used in place of a polyU sequence to deactivate sgRNA activity. The non-canonical terminator sequences are targeted by Cas9 to insert a single nucleotide which disrupts the terminator sequence. A hairpin place 10 nucleotides upstream of the terminator sequence is used to enhance termination frequency.

### Example 5: Multiple Termination Sequences

The purpose of examining multiple termination sequences is to invent a more effective transcriptional termination sequence for small RNA transcribed by RNA Pol III. The concept is that there is a low level of readthrough transcription through polyT tracts of even 10 nt (SEQ ID NO: 90), and extending the length of the tract provides diminishing returns, because the low level readthrough is not decreased substantially and longer polyT tracts pose functional problems for synthesis and stability of plasmid DNA. By contrast, having multiple copies (e.g. two) of a polyT tract could develop multiplicative effects in terms of terminating transcription if each copy causes the same likelihood of termination. The experimental approach was to evaluate the importance of the sequence between multiple (e.g. two) polyT (e.g. 8 nt) tracts. Two different intervening sequences were evaluated: one comprising DNA encoding a 5S ribosomal RNA and the second encoding a sequence predicted to have no secondary RNA structure (e.g., see SEQ ID NOs: 36 and 45 in Table 1 and Table 2 for a non polyT “linear sequence” disposed between two polyT tracts).

### Experimental Detail

Cells (e.g. HEK 293 cells) harboring a genomic expression transgene (e.g. EGFP) were transfected with mixtures of plasmid DNA (e.g. containing a Cas9-VPR expression plasmid and combinations of proGuide plasmids, aGuide plasmids and sgRNA plasmids) to test the effects of multiple polyT tract configurations. A number of proGuides (e.g. single polyT, linear multipolyT, 5S RNA multipoly T) were tested. All proGuide variants had the same spacer sequence targeting the disruption of the transgene (e.g. EGFP). The frequency of cells that lost signal (e.g. GFP fluorescence) was used to assess activity of guide RNA.

### Results

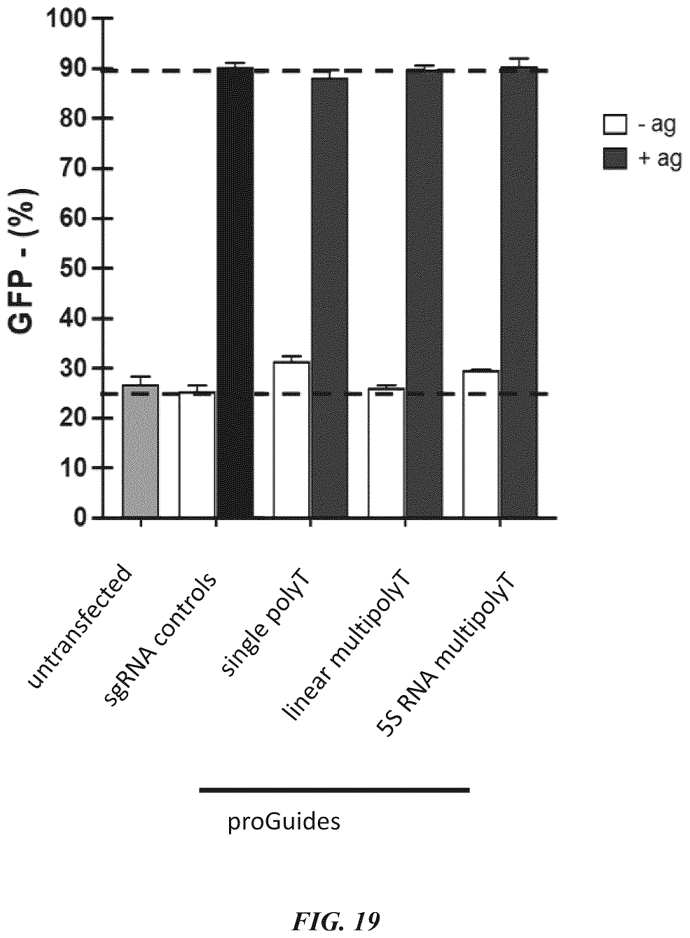

In side by side comparisons, proGuides containing multiple (e.g. two) 8 nt polyT tracts separated by the linear sequence displayed background activity that was indistinguishable from the negative control transfection (white bar; no sgRNA, no proGuide) (FIG. 19). The proGuide containing the polyT tracts separated by the 5s RNA sequence (e.g. 5SRNA multipolyT) displayed detectable background activity, making it a less efficient method of inactivating guide RNA compared to using linear multipolyT. With the addition of the aGuide, the proGuides harboring multiple polyT tracts were converted to an active matureGuide state with a frequency that was indistinguishable from the activity of an sgRNA directly targeting the gene (e.g. EGFP).

### Discussion

The addition of a second polyT tract improved the performance of transcriptional termination in proGuides. However, the effect was dependent on the sequence used to separate the two polyT tracts. With the inclusion of a “linear” sequence between the polyT tracts, virtually no residual guide RNA activity was detected.

### Example 6: Multi-Step Forward and Reverse Cascades

Systems and methods as provided herein (e.g based on a polynucleotide sequence encoding an activatable sgRNA, which polynucleotide sequence comprising one or more polyT sequence) can be utilized to induce a sequentially delimited multi-step cascade effect, whereby the expression of the endogenous gene product can be activated at any step in the cascade.

For example, the multi-step cascade effect can be a 10-step cascade effect, such as a 10-step forward cascade or a 10-step reverse cascade.

### Experimental Details

In summary, the experiment begins with making mixtures of plasmid DNAs encoding the components of the proGuide cascade, proceeds by introducing those DNA into cells (e.g. HEK 293 cells) via nucleofection, and concludes by evaluating the effects on activation of a target gene product at various time points using flow cytometry detection of the cell surface gene product (e.g. CXCR4).

Essential components of mixes of plasmid DNA (e.g. a Cas9-VPR expression plasmid and a GFP expression plasmid) are used to identify transfected cells. To construct combinations of plasmids to activate an endogenous gene at different steps in a cascade of proGuides, mixtures of cascade plasmid DNA used components described in Table 1 and Table 2. Core cascade plasmids were progressively included in transfection mixtures to add additional steps in a cascade as follows. For example, the first step (e.g. Step 1) condition included no proGuides and an sgRNA with a spacer sequence targeting the 5′ and 3′ cut sites within the second step (e.g. Step 2) proGuide plasmid. The second step (e.g. Step 2) condition included all the plasmids in the first step (e.g. Step 1) condition+ proGuide plasmid described for the second step (e.g. Step 2). The third step (e.g. Step 3) condition included all of the plasmids in the second step (e.g. Step 2) condition+ the proGuide described for the third step (e.g. Step 3), and so on. To keep the mass of each proGuide plasmid DNA constant and the mass of total DNA constant for all transfections, a genetically inert plasmid DNA (e.g. pUC19) was used as a “filler” for conditions with fewer proGuide plasmids.

To activate the expression of the endogenous gene product (e.g. CXCR4), a 14 nt spacer sequence was used to target Cas9-VPR to the promoter region of the gene (e.g. CXCR4). For activation at the first step (e.g. Step 1), the gene (e.g. CXCR4) activation was stimulated by an sgRNA harboring the relevant spacer for the gene (e.g. 14 nt CXCR4 spacer). For subsequent steps, a proGuide plasmid with the relevant spacer for the gene (e.g. 14 nt CXCR4 spacer) was added to the plasmid DNA mix. By matching the 5′ and 3′ cut sites for a particular step in a cascade with the 5′ and 3′ cut sites in the gene (e.g. CXCR4)-activating proGuide, activation of the gene (e.g. CXCR4) was effectively programmed to occur at one particular step in the cascade for each condition/mixture of plasmid DNA.

Mixtures of plasmid DNA were introduced into cells (e.g. HEK 293 cells) using standard procedures with a nucleofection system (e.g. Lonza 4D). Transfected cells were plated (e.g. in multiwell tissue culture plates) and maintained using standard mammalian tissue culture methods. At specified time points (e.g. 12, 24, 36, 48 and 72 hours) after nucleofection, cells were processed for flow cytometry and detection of cell surface expression of gene product (e.g. CXCR4). For each condition, independent replicates (e.g. n=4) (nucleofections) were examined by flow cytometry.

### Results

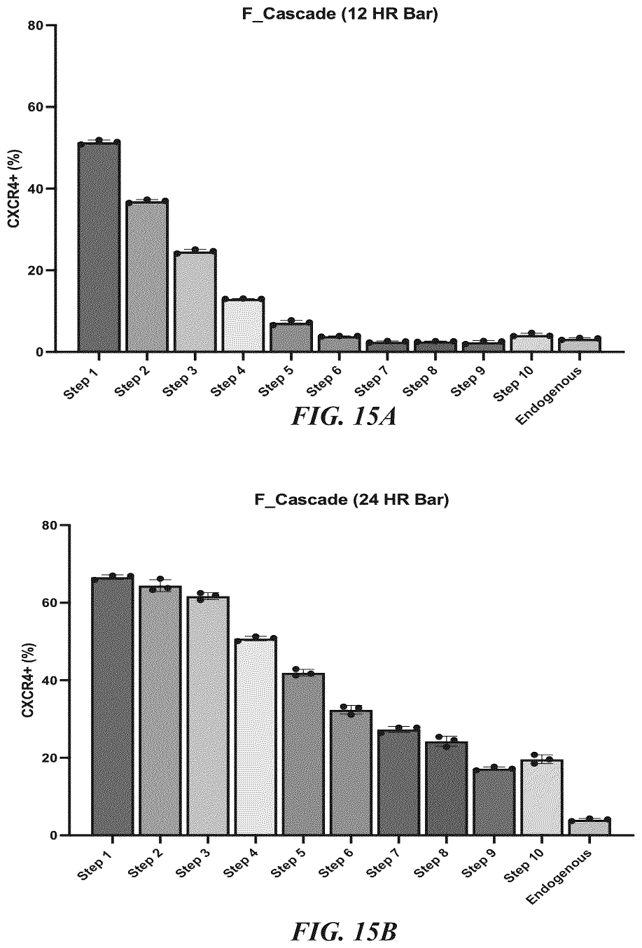

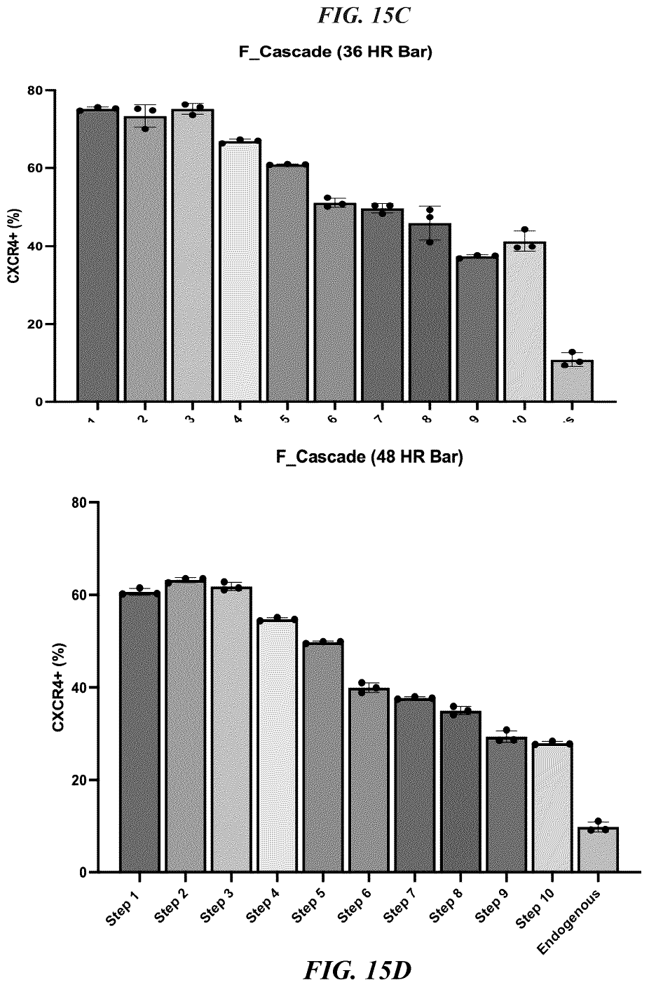

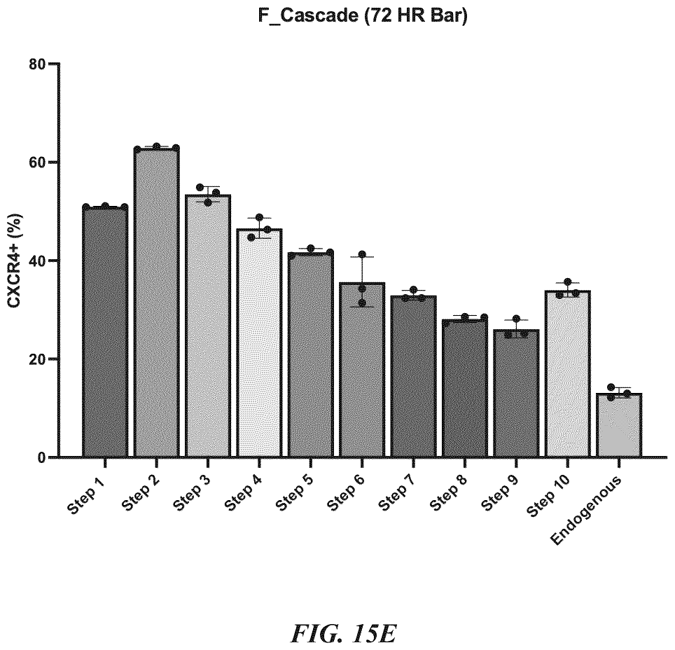

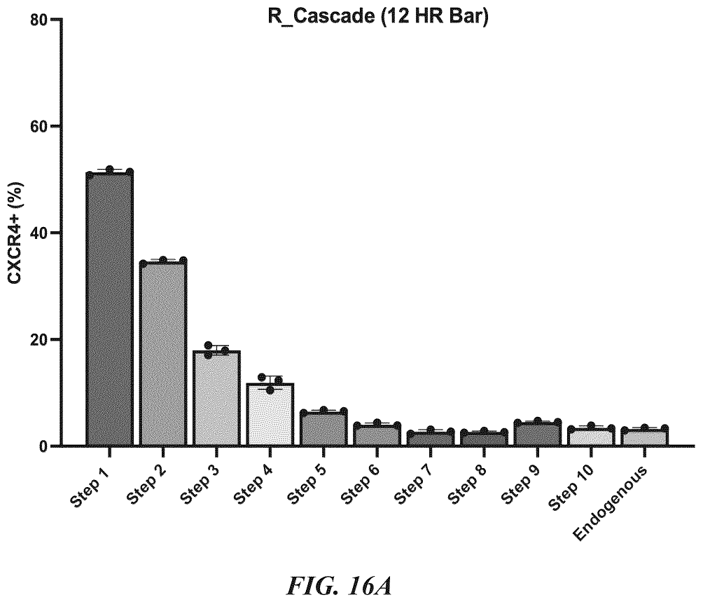

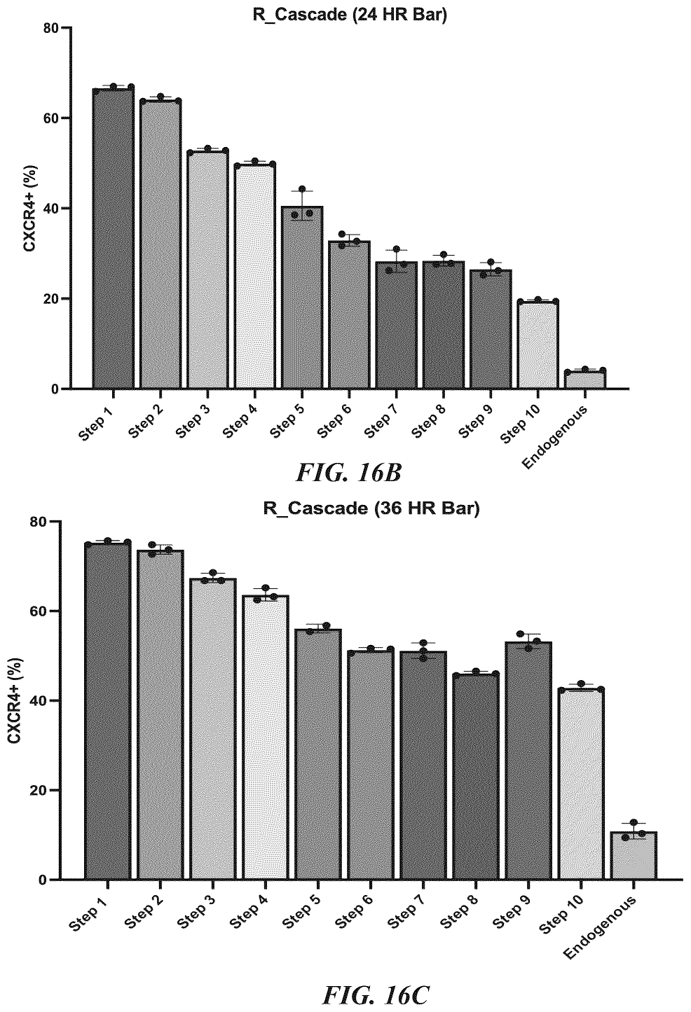

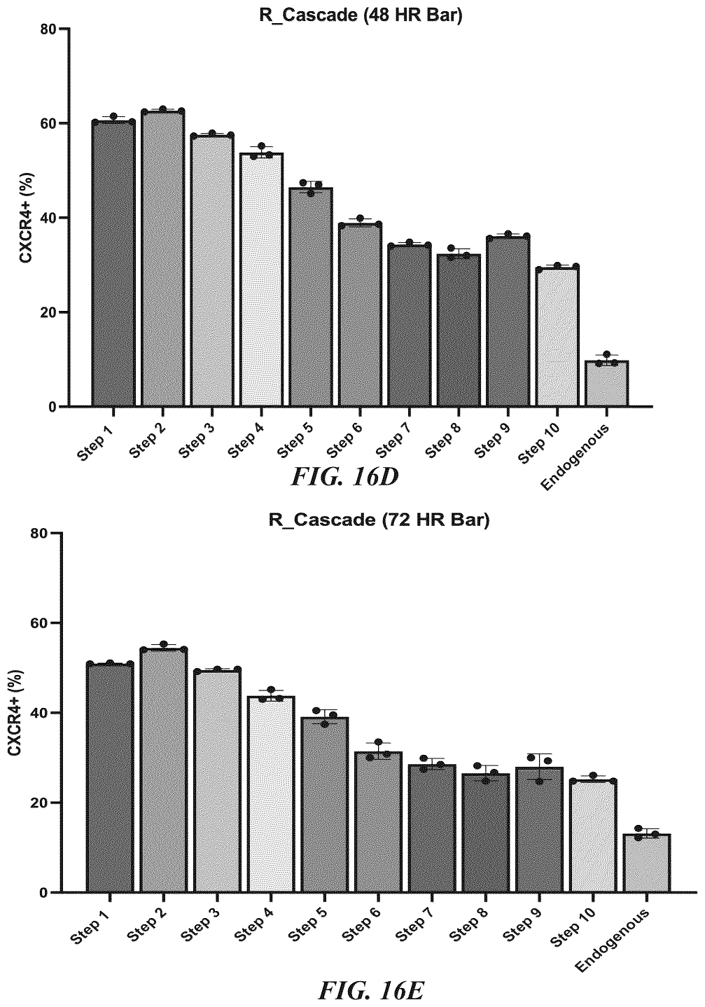

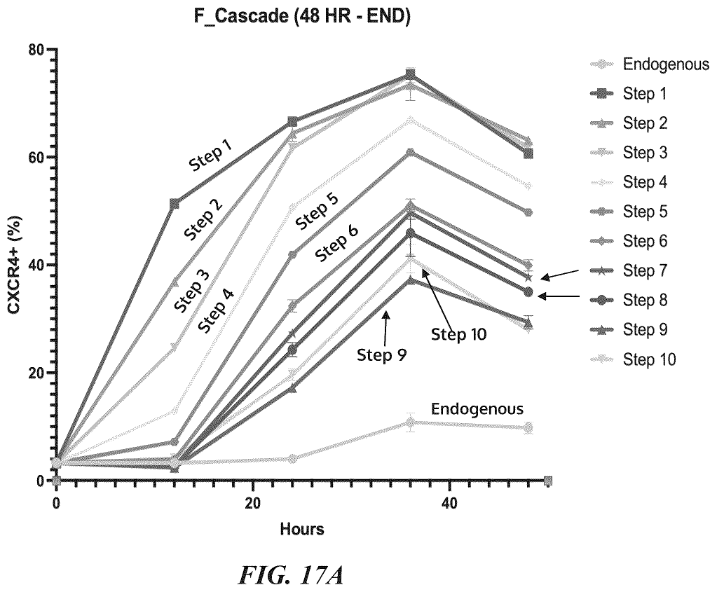

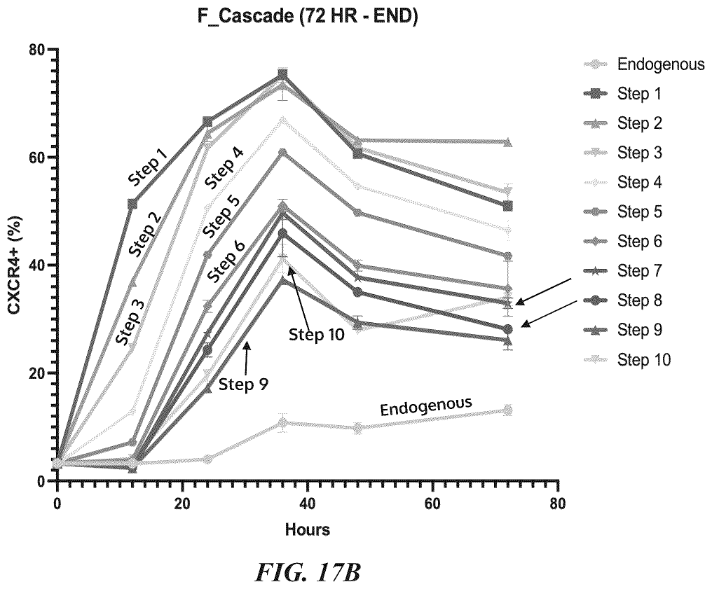

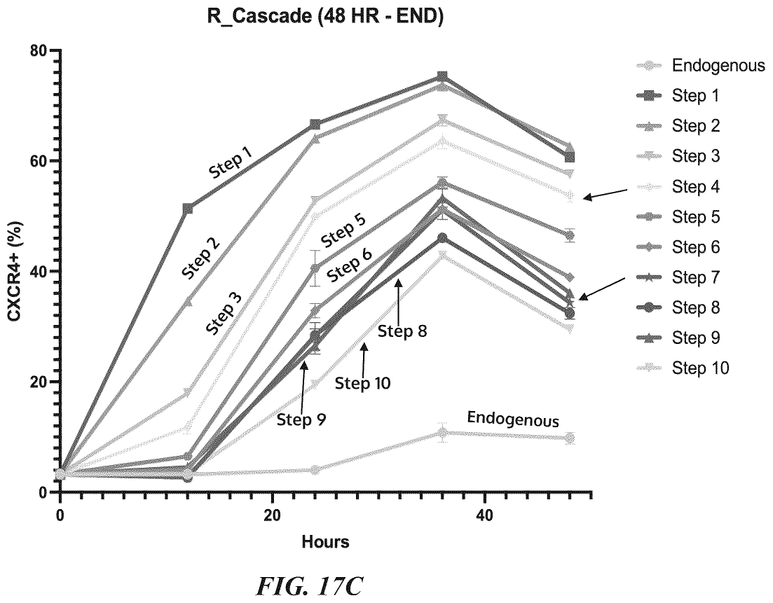

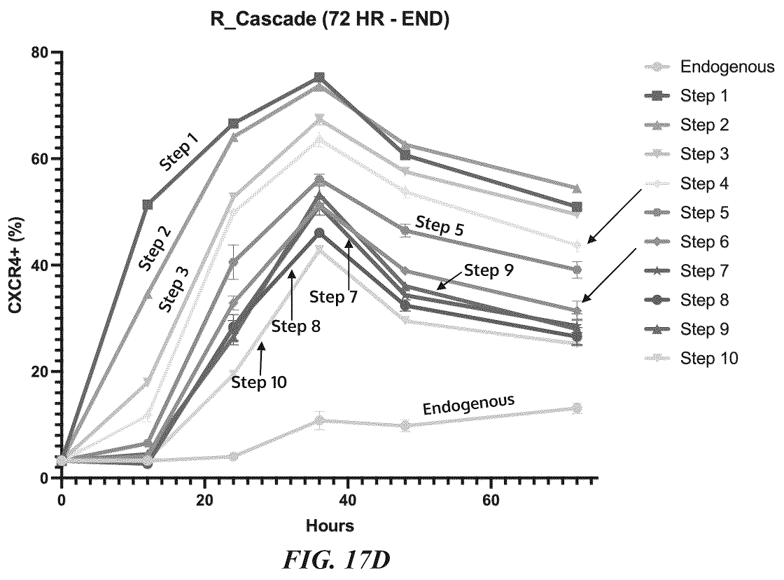

As expected, cell surface expression of gene (e.g. CXCR4) was activated by the combination of Cas9-VPR and an sgRNA targeting the promoter region of the endogenous gene (e.g. CXCR4) (e.g. Step 1; FIGS. 15A-17D). The first step (e.g. Step 1) sgRNA stimulated the greatest level of gene (e.g. CXCR4) increase within a first time point (e.g. 12 hr). By contrast, each proGuide-mediated step (e.g. Step 2-10) displayed a delay in activation of the gene (e.g. CXCR4) relative to the sgRNA. Importantly, proGuide mediated steps also displayed a delay in activation relative to earlier proGuide mediated steps. For example, activation of the gene (e.g. CXCR4) programmed at the third step (e.g. Step 3) displayed a delay relative to activation programmed at the second step (e.g. Step 2), activation at the fourth step (e.g. Step 4) was delayed relative to activation at the third step (e.g. Step 3), and so on. The programmed delay of later steps occurring after earlier steps was generally consistent in both Forward cascades (FIGS. 15A-15E, FIGS. 17A-17B) and Reverse cascades (FIGS. 16A-16E, FIGS. 17C-17D).

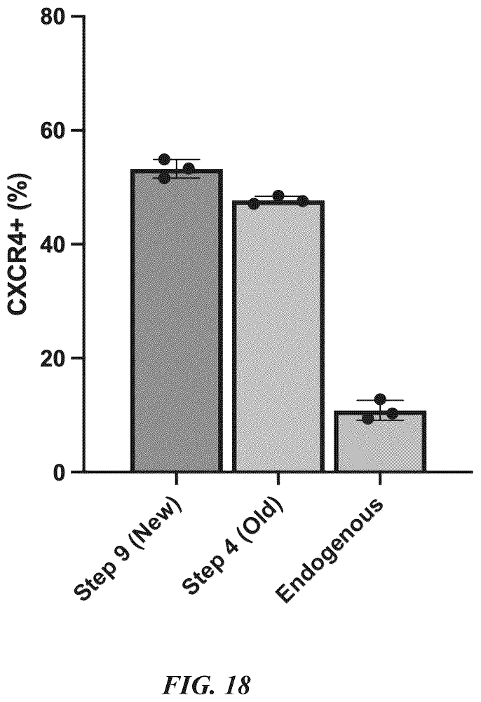

The level of activity progressively declines slightly after each step in the cascade. By Step 7, a plateau appeared to be reached such that the activity at Steps 7-10 was similar after 72 hours (FIG. 16E). Compared to previous versions of the proGuide technology, these cascades are significantly improved. One example of the improvement is that the highest activity of a 4-step cascade using the previous technology was lower than the step 9 level with the new technology in a side by side comparison (FIG. 18).

It was unknown if the sequence composition of the spacer region and that of the cut sites could affect the activity of one another. For example, it was possible that some spacer sequences could interfere with conversion of proGuides or generate matureGuides with inferior activity. To test this possibility, we rearranged the configuration of spacers and cut sites within individual proGuides to form two cascades; the order of events was changed in the Reverse cascade relative to the Forward cascade such that cut site sequences used to go from the first step to the second step (e.g. Step 1 to 2) in the Forward cascade are used to go from Step 9 to 10 in the Reverse cascade, Step 2 to 3 in Forward cascade is used for Step 8 to 9 in Reverse cascade, and so on (Table 1,2). Comparing the activation of genes (e.g. CXCR4) via Forward cascade versus Reverse cascade revealed remarkably few differences in kinetics or levels of activity between the two (FIGS. 15A-17D). These results are consistent with the progression of cascades from one step to the next being governed primarily by the effectiveness of the cut site sequence. Thus, when only high efficiency cut site sequences are used, they are likely to be nearly interchangeable in where they can be used to generate a cascade of proGuides.

Discussion: Two critical parameters for synthetic biology solutions to providing sequential genetic instruction are the efficiency of the system (e.g. percent of cells that complete intended instructions) and the sophistication of the system (e.g. the number of steps that can be encoded). The latest development of proGuide technologies deliver efficiency and sophistication that substantially exceed those of other synthetic biology systems all while retaining the ability to activate essentially any combination of endogenous gene products.

The efficiency of the system is illustrated by comparison of activation of endogenous gene (e.g. CXCR4) expression at the first step (e.g. Step 1) relative to the gold standard of an sgRNA activating the gene (e.g. CXCR4). For each consecutive step in a cascade, over 95% of the cells continue to activate the next step in the cascade. The sophistication of the system is illustrated by completion of multi-step (e.g. 10-step) cascades. The number of steps in a sequential process is unprecedented and compares to traditional methods of using conditional gene activation methods to achieve two steps of activation. The proGuide cascade system progresses autonomously once it is introduced into cells via transfection of plasmid DNA. Thus, it does not require conditional activation (e.g. doxycycline or cumate induction) to be applied by altering culture conditions. Moreover, because it is entirely encoded by plasmid DNA, the proGuide cascade system does not involve nor require gene editing or mutation of host cells for it execute epigenetic programming of cells.

TABLE 1
Example of a heterologous genetic circuit for testing a multi-step cascade
(e.g., a 10-step forward cascade).

| | | Upstream cut | | Downstream | |
|---|---|---|---|---|---|
| Step | Stem | site (e.g., | | cut site (e.g., | Spacer |
| # | name | 5' cut site) | proUnit | 3' cut site) | sequence |
| 1 | NA | NA | NA | NA | SEQ ID |
| (sgRNA) | | | | | NO: 72 |
| | | | | | TAGCTACC |
| | | | | | GATGTCGA |
| | | | | | GTGT |
| 2 | 1 | SEQ ID NO: 17 | SEQ ID NO: 36 | SEQ ID NO: | SEQ ID |
| | | CCTACACTCGACATCG | TTTTTTTTcagcca | 54 | NO: 73 |
| | | GTAGCTA | actccaaTTTTTTT | TAGCTACCG | ATTACTCG |
| | | | T | ATGTCGAGT | AACGTTCC |
| | | | | GTAGG | GCCA |
| 3 | 3 | SEQ ID NO: 18 | SEQ ID NO: 36 | SEQ ID NO: | SEQ ID |
| | | CCCTGGCGGAACGTT | | 55 | NO: 74 |
| | | CGAGTAAT | | ATTACTCGA | GCGCACGA |
| | | | | ACGTTCCGC | CCACTATC |
| | | | | CAGGG | GTGT |
| 4 | 7 | SEQ ID NO: 19 | SEQ ID NO: 36 | SEQ ID NO: | SEQ ID |
| | | CCTACACGATAGTGGT | | 56 | NO: 75 |
| | | CGTGCGC | | GCGCACGA | ACTCGTTC |
| | | | | CCACTATCG | GATAGAGA |
| | | | | TGTAGG | GTTC |
| 5 | 6 | SEQ ID NO: 20 | SEQ ID NO: 36 | SEQ ID NO: | SEQ ID |
| | | CCCGAACTCTCTATCG | | 57 | NO: 76 |
| | | AACGAGT | | ATAGAGAGT | TCGATCGT |
| | | | | TCGGG | GCCA |
| | | | | SEQ ID NO: | SEQ ID |
| 6 | 12 | SEQ ID NO: 21 | SEQ ID NO: 36 | 58 | NO: 77 |
| | | CCATGGCACGATCGA | | CCTCCGTGT | GCTCAGTC |
| | | CACGGAGG | | CGATCGTGC | GCGAATGA |
| | | | | CATGG | GCTT |
| 7 | 13 | SEQ ID NO: 22 | SEQ ID NO: 36 | SEQ ID NO: | SEQ ID |
| | | CCAAAGCTCATTCGC | | 59 | NO: 78 |
| | | GACTGAGC | | GCTCAGTCG | TAGCTCCC |
| | | | | CGAATGAGC | GTCCGTAG |
| | | | | TTTGG | ACGT |
| 8 | 8 | SEQ ID NO: 23 | SEQ ID NO: 36 | SEQ ID NO: | SEQ ID |
| | | CCGACGTCTACGGAC | | 60 | NO: 79 |
| | | GGGAGCTA | | TAGCTCCCG | TGCGTCGT |
| | | | | TCCGTAGAC | CTACTACCT |
| | | | | GTCGG | CTC |
| 9 | 11 | SEQ ID NO: 24 | SEQ ID NO: 36 | SEQ ID NO: | SEQ ID |
| | | CCCGAGAGGTAGTAG | | 61 | NO: 80 |
| | | ACGACGCA | | TGCGTCGTC | TGCTACGC |
| | | | | TACTACCTC | ATACGTGA |
| | | | | TCGGG | CGAC |
| 10 | 10 | SEQ ID NO: 26 | SEQ ID NO: 36 | SEQ ID NO: | NA(gene |
| | | CCCGTCGTCACGTATG | | 62 | specific) |
| | | CGTAGCA | | TGCTACGCA | |
| | | | | TACGTGACG | |
| | | | | ACGGG | |

TABLE 2
Example of an additional heterologous genetic circuit for testing a
multi-step cascade (e.g., a 10-step reverse cascade, based on having
the order of the downstream/upstream cut site pairs reversed
from the heterologous genetic circuit in Table 1).

| | | Upstream cut | | Downstream | |
|---|---|---|---|---|---|
| | | site | | cut site | |
| | Stem | (e.g., 5' | | (e.g., 3' | Spacer |
| Step # | name | cut site) | proUnit | cut site) | sequence |
| 1 | NA | NA | NA | NA | SEQ ID NO: 81 |
| (sgRNA) | | | | | TGCTACGCA |
| | | | | | TACGTGACG |
| | | | | | AC |
| 2 | 10 | SEQ ID NO: 27 | SEQ ID NO: 45 | SEQ ID NO: 63 | SEQ ID NO: 82 |
| | | CCCGTCGTCACGTATG | TTTTTTTTcagccaa | TGCTACGCAT | TGCGTCGTC |
| | | CGTAGCA | ctccaaTTTTTTTT | ACGTGACGA | TACTACCTC |
| | | | | CGGG | TC |
| 3 | 11 | SEQ ID NO: 28 | SEQ ID NO: 45 | SEQ ID NO: 64 | SEQ ID NO: 83 |
| | | CCCGAGAGGTAGTAGA | | TGCGTCGTCT | TAGCTCCCG |
| | | CGACGCA | | ACTACCTCTC | TCCGTAGAC |
| | | | | GGG | GT |
| 4 | 8 | SEQ ID NO: 29 | SEQ ID NO: 45 | SEQ ID NO: 65 | SEQ ID NO: 84 |
| | | CCGACGTCTACGGACG | | TAGCTCCCGT | GCTCAGTCG |
| | | GGAGCTA | | CCGTAGACG | CGAATGAGC |
| | | | | TCGG | TT |
| 5 | 13 | SEQ ID NO: 30 | SEQ ID NO: 45 | SEQ ID NO: 66 | SEQ ID NO: 85 |
| | | CCAAAGCTCATTCGCG | | GCTCAGTCG | CCTCCGTGT |
| | | ACTGAGC | | CGAATGAGC | CGATCGTGC |
| | | | | TTTGG | CA |
| 6 | 12 | SEQ ID NO: 31 | SEQ ID NO: 45 | SEQ ID NO: 67 | SEQ ID NO: 86 |
| | | CCATGGCACGATCGAC | | CCTCCGTGTC | ACTCGTTCG |
| | | ACGGAGG | | GATCGTGCC | ATAGAGAGT |
| | | | | ATGG | TC |
| 7 | 6 | SEQ ID NO: 32 | SEQ ID NO: 45 | SEQ ID NO: 68 | SEQ ID NO: 87 |
| | | CCCGAACTCTCTATCG | | ACTCGTTCG | GCGCACGA |
| | | AACGAGT | | ATAGAGAGT | CCACTATCG |
| | | | | TCGGG | TGT |
| 8 | 7 | SEQ ID NO: 33 | SEQ ID NO: 45 | SEQ ID NO: 69 | SEQ ID NO: 88 |
| | | CCTACACGATAGTGGT | | GCGCACGAC | ATTACTCGA |
| | | CGTGCGC | | CACTATCGTG | ACGTTCCGC |
| | | | | TAGG | CA |
| 9 | 3 | SEQ ID NO: 34 | SEQ ID NO: 45 | SEQ ID NO: 70 | SEQ ID NO: 89 |
| | | CCCTGGCGGAACGTTC | | ATTACTCGAA | TAGCTACCG |
| | | GAGTAAT | | CGTTCCGCC | ATGTCGAGT |
| | | | | AGGG | GT |
| 10 | 1 | SEQ ID NO: 35 | SEQ ID NO: 45 | SEQ ID NO: 71 | |
| | | CCTACACTCGACATCG | | TAGCTACCG | NA(gene |
| | | GTAGCTA | | ATGTCGAGT | specific) |
| | | | | GTAGG | |

### Example 7: Examination of Conversion to matureGuide RNA Using DNA Sequencing

Systems and methods herein can have one or more mechanistic pathways. An important parameter in synthetic biology solutions is the efficiency of conversion at certain steps. In some cases, the conversion can be the conversion of a proGuide to a matureGuide. In some cases, the architecture of the proGuide can influence the efficiency of conversion to a matureGuide.

To examine the DNA repair process required for the conversion of a proGuide to a matureGuide, the RNA sequence of matureGuide RNA transcripts was characterized in cells. The sequencing experiment was used to elucidate potential causes underlying the increased efficiencies observed in Type 2 and 3 over Type 1. Type 1 refers to the proGuide architecture of FIGS. 1A-1B (e.g., having a polyT having a length less than 7). Type 2 and Type 3 architectures are illustrated in FIG. 22A and FIG. 22B, respectively. Example of differences between Type 1 vs Type 2 and 3 include the removal of elements from Type 1 (insulator, restriction site, ribozyme) and the orientation of the cut sites from a direct repeat in Type 1 to inverted repeat in Type 2 and 3. In addition, length of polyT in Type 1 proGuide (e.g., shorter than 7) is less than length of polyT in Type 2 or 3 proGuide (e.g., longer than or equal to 7, such as 8 or 9). Notably, Type 3 incorporates multiple (e.g. two) polyT sequences into its architecture. The experimental procedure for the characterization involved the transfection of cells (e.g. HEK 293 cells) with plasmid DNA encoding proGuides with the same cut site sequences, but different proGuide architectures. For each transfection a proGuide was co-transfected with an expression plasmid (e.g. Cas9-VPR) and an sgRNA targeting the cut site of the proGuide plasmid (i.e. an aGuide). RNA was extracted at a specified time point (e.g. 36 hours) after transfection, converted to cDNA, and amplified using guide RNA specific primers such that only RNA molecules with the proGuide spacer and complete scaffold (i.e. tetraloop, hairpin 1, hairpin 2) would be sequenced.

### Results and Discussion

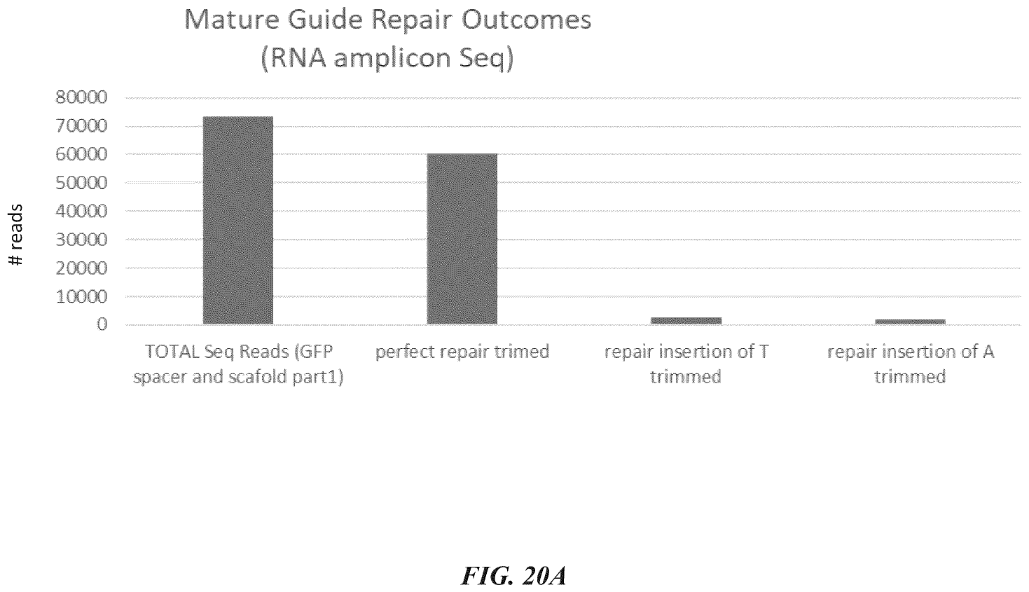

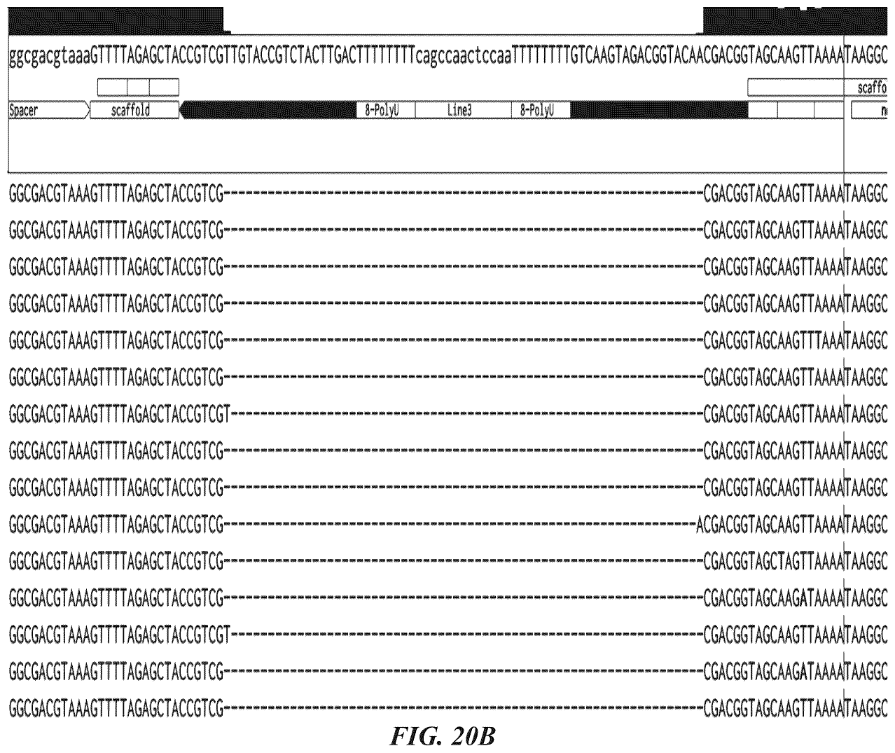

FIG. 20A shows the frequency of RNA corresponding to a perfect NHEJ repair outcome for a Type 3 proGuide. The perfect repair outcome is defined as a sequence in which the Cas9 cut sites are ligated together without an additional insertion or deletion of nucleotides. FIG. 20B shows the DNA sequences observed from the experiment for the Type 3 proGuide also described in FIG. 20A. Note that the top sequence is an example of a perfect NHEJ repair of . . . TACCGTCG------------CGACGGTA . . . (the PAM sequence are underlined here for reference) (SEQ ID NO: 105). The sequencing results showed that the perfect repair outcome represented the vast majority of matureGuide RNA in cells, and the next frequent outcomes of a single insertion of an A or T (corresponding to a U in the RNA) were infrequently observed.

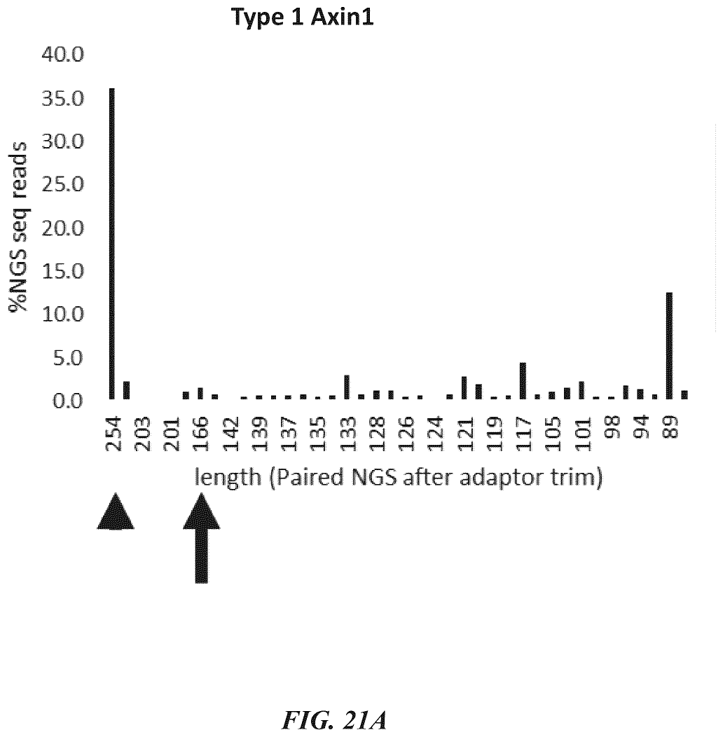

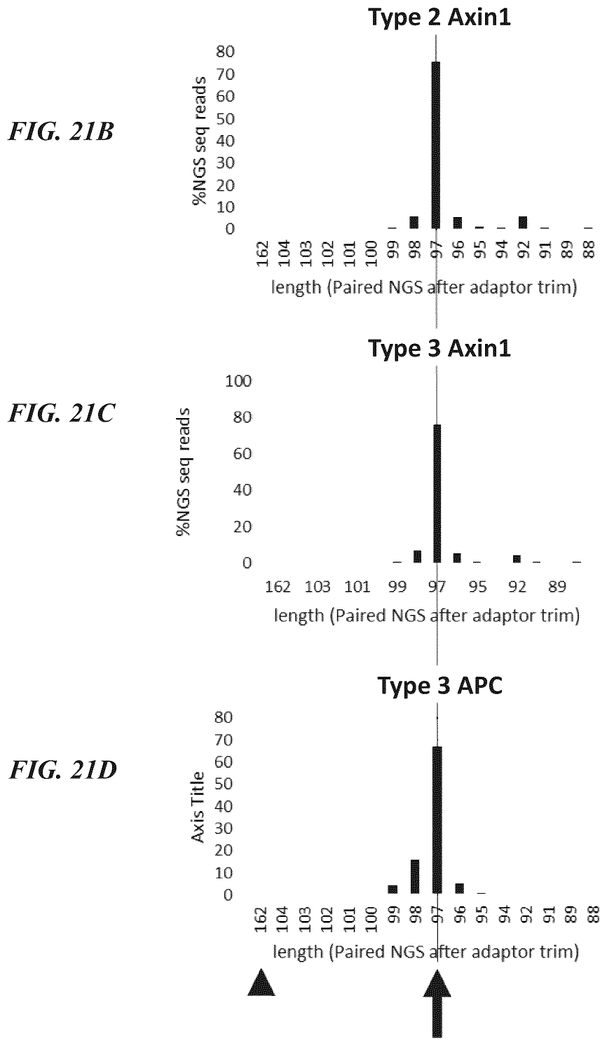

Using the DNA sequencing approach to compare different generations of proGuides demonstrated significant improvements. FIGS. 21A-21D show the size distribution of mapped sequencing reads for different proGuides. For example, in FIGS. 21A-21D, the nomenclature can denote the type of the proGuide (e.g., Type 1, Type 2, or Type 3), followed by the nature of the cut site sequence within the proGuide to transform the proGuide to a matureGuide. Those labeled “Axin1” all shared the same cut site sequence, although the cut sites in Type 1 were arranged in a direct repeat orientation rather than the inverted repeat orientation in Type 2 and 3. The distribution of RNA sizes indicates that the original architecture allowed not only substantial readthrough transcription and existence of full-length proGuide RNA (triangle), but the perfect NHEJ repair outcome (arrow) was a minority occurrence relative to repair outcomes resulting in other sizes of RNAs (FIG. 21A). Type 2 (FIG. 21B) and Type 3 (FIG. 21C) displayed similar distributions of matureGuide RNA sizes, relative to one another, corresponding predominantly to the perfect NHEJ repair outcome (arrow). A proGuide possessing a less than optimal cut site (e.g. Type 3 APC) was repaired with the slightly lower frequency of perfect NHEJ repair outcomes (FIG. 21D). Note that the sequencing assay does not have the ability to assess the activity of repair events, only the outcomes of those repair events leading to a full length matureGuide RNA molecule.

### EMBODIMENTS

The following non-limiting embodiments provide illustrative examples of the invention, but do not limit the scope of the invention.

Embodiment 1. A system for regulating expression or activity of a target gene, the system comprising:
a polynucleotide sequence encoding a guide nucleic acid molecule, wherein the guide nucleic acid molecule exhibits specific affinity to the target gene, to regulate the expression or the activity of the target gene,
wherein the polynucleotide sequence comprises a domain that (i) corresponds to a tetraloop region of the guide nucleic acid molecule, and (ii) comprises a polyT sequence, wherein the polyT sequence is sufficient to reduce expression of the guide nucleic acid molecule, thereby regulating expression or activity of the target gene,
optionally wherein:
(1) a size of the polyT sequence is greater than or equal to a threshold length, wherein the threshold length is sufficient to reduce expression of the guide nucleic acid molecule from the polynucleotide sequence,
   further optionally wherein:
   (a) the polyT sequence comprises at least 6 T; and/or
   (b) the polyT sequence comprises at least 7 T; and/or
   (c) the polyT sequence comprises at least 8 T; and/or
   (d) the polyT sequence comprises at least 9 T or at least 10 T; and/or
   (e) the polyT sequence comprises between 6 T and 15 T; and/or
(2) the polyT sequence comprises one or more additional nucleotides that are not T; and/or
(3) the polyT sequence flanks an intervening sequence that is not a polyT sequence; and/or
(4) the polynucleotide sequence further comprises an insulator sequence, wherein the insulator sequence is located adjacent to the polyT sequence, and wherein the insulator sequence comprises a sequence which is targetable by a gene editing moiety,
   further optionally wherein:
   (a) the insulator sequence is fully complementary; and/or
   (b) the insulator sequence comprises a non-complementary stem region.

Embodiment 2. A system for regulating expression or activity of a target gene, the system comprising:
a polynucleotide sequence encoding a guide nucleic acid molecule, wherein the guide nucleic acid molecule is characterized by (i) exhibiting specific affinity to the target gene, to regulate the expression or activity of the target gene, and (ii) has a size of at least about 12 nucleotides, wherein the polynucleotide sequence comprises a polyX sequence having a threshold length that is greater than or equal to five, such that the polyX sequence is sufficient to reduce expression of the guide nucleic acid molecule from the polynucleotide sequence, wherein the polyX sequence does not correspond to a terminal domain of the guide nucleic acid molecule,
optionally wherein:
(1) the poly X sequence comprises at least 6 X; and/or
(2) the poly X sequence comprises at least 7 X; and/or
(3) the polyX sequence comprises at least 8 X; and/or
(4) the polyX sequence comprises at least 9 X or at least 10 X; and/or
(5) the polyX sequence comprises between 6X and 15X; and/or
(6) the poly X sequence is a polyT sequence; and/or
(7) the polyX sequence is located in a domain corresponding to a tetraloop region of the guide nucleic acid molecule; and/or
(8) the polyX sequence is located in a domain corresponding to a hairpin region of the guide nucleic acid molecule; and/or
(9) the guide nucleic acid molecule has a size of at most 300 nucleotides.

Embodiment 3. The system of Embodiment 1 or Embodiment 2, wherein the system further comprises a gene editing moiety configured to make at least one edit to the polyT sequence or the polyX sequence, wherein the at least one edit effects transcription of the guide nucleic acid molecule,
optionally wherein:
(1) the at least one edit is an insertion; and/or
(2) the at least one edit is a deletion; and/or
(3) the at least one edit is an excision of the polyX sequence; and/or
(4) the excision of the polyX sequence is accomplished using two cut sites which flank the poly X sequence; and/or
(5) the at least one edit comprises microhomology-mediated end joining (MMEJ) repair; and/or
(6) the at least one edit enhances expression of the guide nucleic acid molecule from the polynucleotide sequence as compared to that in absence of the gene editing moiety; and/or
(7) the gene editing moiety comprises a Cas protein; and/or
(8) the poly X sequence comprises one or more additional nucleotides that are not X; and/or
(9) the polyX sequence flanks an intervening sequence that is not a polyX sequence.

Embodiment 4. The system of any one of Embodiments 1-3, optionally wherein:
(1) the polynucleotide sequence comprises (i) a first region encoding the guide nucleic acid molecule, and (ii) a second region encoding an endonuclease recognition site, wherein the second region is disposed adjacent to the first region; and/or
(2) the polyT sequence or the polyX sequence is at least 80 nucleotides away from the 3′ end of the polynucleotide sequence; and/or
(3) the polyT sequence or the polyX sequence is at least 14 nucleotides away from the 5′ end of the polynucleotide sequence; and/or
(4) the polynucleotide sequence further comprises at least one filler sequence adjacent to the polyT sequence or the poly X sequence,
   further optionally wherein:
   (i) the at least one filler sequence comprises a first filler sequence and a second filler sequence, and wherein the polyT sequence or the polyX sequence is flanked by the first filler sequence and the second filler sequence; and/or
(5) the system further comprises an endonuclease capable of forming a complex with the guide nucleic acid molecule, wherein the complex effects regulation of the expression or activity of the target gene,
   further optionally wherein:
   (i) the endonuclease comprises a Cas protein; and/or
(6) the guide nucleic acid molecule does not comprise a ribozyme; and/or
(7) the polynucleotide sequence comprises the structure:

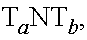

wherein: (i) Tₐ is a first poly T sequence; (ii) T_{b} is a second poly T sequence; (iii) a and b are integers greater than or equal to 4; and (iv) Nis an intervening sequence comprising at least one nucleobase that is not T,
   further optionally wherein a and b are integers greater than or equal to 7; and/or
(8) the polynucleotide sequence comprises the structure:

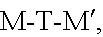

wherein: (i) T is the polyT sequence; (ii) M and M′ are polynucleotide sequences that are at least partially complementary to one another; and (iii) “-” is a polynucleotide linker or absent; and/or
(9) a polynucleotide sequence of M and an additional polynucleotide sequence M′ exhibit at least about 50% sequence identity to the pair of polynucleotide sequences, respectively, selected from the group consisting of (1) SEQ ID NO: 17 and SEQ ID NO: 54; (2) SEQ ID NO: 18 and SEQ ID NO: 55; (3) SEQ ID NO: 19 and SEQ ID NO: 56; (4) SEQ ID NO: 20 and SEQ ID NO: 57; (5) SEQ ID NO: 21 and SEQ ID NO: 58; (6) SEQ ID NO: 22 and SEQ ID NO: 59; (7) SEQ ID NO: 23 and SEQ ID NO: 60; (8) SEQ ID NO: 24 and SEQ ID NO: 61; (9) SEQ ID NO: 26 and SEQ ID NO: 62; (10) SEQ ID NO: 27 and SEQ ID NO: 63; (11) SEQ ID NO: 28 and SEQ ID NO: 64; (12) SEQ ID NO: 29 and SEQ ID NO: 65; (13) SEQ ID NO: 30 and SEQ ID NO: 66; (14) SEQ ID NO: 31 and SEQ ID NO: 67; (15) SEQ ID NO: 32 and SEQ ID NO: 68; (16) SEQ ID NO: 33 and SEQ ID NO: 69; (17) SEQ ID NO: 34 and SEQ ID NO: 70; and (18) SEQ ID NO: 35 and SEQ ID NO: 71, and a complementary sequence pair thereof,
   further optionally wherein:
   (i) the polynucleotide sequence of M and the additional polynucleotide sequence M′ exhibit at least about 60% sequence identity to the pair of polynucleotide sequences, respectively, selected from the group consisting of (1)-(18); and/or
   (ii) the polynucleotide sequence of M and the additional polynucleotide sequence M′ exhibit at least about 80% sequence identity to the pair of polynucleotide sequences, respectively, selected from the group consisting of (1)-(18).

Embodiment 5. A method for regulating expression or activity of a target gene in a cell, the method comprising:
contacting the cell with a polynucleotide sequence encoding a guide nucleic acid molecule, wherein the guide nucleic acid molecule exhibits specific affinity to the target gene, to regulate the expression or the activity of the target gene,
wherein the polynucleotide sequence comprises a domain that (i) corresponds to a tetraloop region of the guide nucleic acid molecule, and (ii) comprises a polyT sequence, wherein the polyT sequence is sufficient to reduce expression of the guide nucleic acid molecule, thereby regulating expression or activity of the target gene,
optionally wherein:
(1) a size of the polyT sequence is greater than or equal to a threshold length, wherein the threshold length is sufficient to reduce expression of the guide nucleic acid molecule from the polynucleotide sequence in the cell; and/or
(2) the polyT sequence comprises at least 6 T; and/or
(3) wherein the polyT sequence comprises at least 7 T; and/or
(4) wherein the polyT sequence comprises at least 8 T; and/or
(5) wherein the polyT sequence comprises at least 9 T or at least 10 T; and/or
(6) wherein the polyT sequence comprises between 6 T and 15 T; and/or
(7) wherein the polyT sequence comprises one or more additional nucleotides that are not T; and/or
(8) wherein the polyT sequence flanks an intervening sequence that is not a polyT sequence; and/or
(10) the polynucleotide sequence further comprises an insulator sequence, wherein the insulator sequence is located adjacent to the polyT sequence, and wherein the insulator sequence comprises a sequence which is targetable by a gene editing moiety,
   further optionally wherein:
   (a) the insulator sequence is fully complementary; and/or
   (b) the insulator sequence comprises a non-complementary stem region.

Embodiment 6. A method for regulating expression or activity of a target gene in a cell, the method comprising:
providing a polynucleotide sequence encoding a guide nucleic acid molecule to the cell, wherein the guide nucleic acid molecule is characterized by (i) exhibiting specific affinity to the target gene, to regulate the expression or activity of the target gene, and (ii) has a size of at least about 12 nucleotides,
wherein the polynucleotide sequence comprises a polyX sequence having a threshold length that is greater than or equal to five, such that the polyX sequence is sufficient to reduce expression of the guide nucleic acid molecule from the polynucleotide sequence, wherein the poly X sequence does not correspond to a terminal domain of the guide nucleic acid molecule,
optionally wherein:
(1) the poly X sequence comprises at least 6 X; and/or
(2) the poly X sequence comprises at least 7 X; and/or
(3) the poly X sequence comprises at least 8 X; and/or
(4) the polyX sequence comprises at least 9X or at least 10 X; and/or
(5) the poly X sequence comprises between 6 and 15 X; and/or
(6) the poly X sequence is a polyT sequence; and/or
(7) the polyX sequence is located in a domain corresponding to a tetraloop region of the guide nucleic acid molecule; and/or
(8) the polyX sequence is located in a domain corresponding to a hairpin region of the guide nucleic acid molecule; and/or
(9) the polyX sequence comprises one or more additional nucleotides that are not X; and/or
(10) the polyX sequence flanks an intervening sequence that is not a polyX sequence.

Embodiment 7. The method of Embodiment 6 or Embodiment 7, optionally wherein, the method further comprises modifying the polyT sequence or the polyX sequence in the polynucleotide sequence, to alter expression level of the guide nucleic acid molecule from the polynucleotide sequence, thereby to effect regulation of the expression or activity of the target gene in the cell,
optionally wherein:
(1) the modifying comprises generating at least one edit to the polyT sequence or the polyX sequence,
   further optionally wherein:
   (a) the at least one edit comprises microhomology-mediated end joining (MMEJ) repair; and/or
   (b) the at least one edit enhances expression of the guide nucleic acid molecule from the polynucleotide sequence; and/or
(2) the at least one edit is an insertion; and/or
(3) the at least one edit is a deletion; and/or
(4) the at least one edit is an excision of the polyX sequence,
   further optionally wherein:
   (a) the excision of the polyX sequence is accomplished using two cut sites which flank the poly X sequence; and/or
(5) the modifying reduces a size of the polyX sequence below the threshold length; and/or
(6) the modifying comprises contacting the polynucleotide sequence with a gene editing moiety.

Embodiment 8. The method of any one of Embodiments 6-8, optionally wherein:
(1) the polynucleotide sequence comprises (i) a first region encoding the guide nucleic acid molecule, and (ii) a second region encoding an endonuclease recognition site, wherein the second region is disposed adjacent to the first region; and/or
(2) the polyT sequence or the polyX sequence is at least 80 nucleotides away from the 3′ end of the polynucleotide sequence; and/or
(3) the polyT sequence or the polyX sequence is at least 14 nucleotides away from the 5′ end of the polynucleotide sequence; and/or
(4) the polynucleotide sequence further comprises at least one filler sequence adjacent to the polyT sequence or the poly X sequence,
   further optionally wherein:
   (a) the at least one filler sequence comprises a first filler sequence and a second filler sequence, and wherein the polyT sequence or the polyX sequence is flanked by the first filler sequence and the second filler sequence; and/or
(5) the guide nucleic acid molecule further comprises an endonuclease recognition site; and/or
(6) the cell is a mammalian cell; and/or
(7) the method further comprises forming a complex with the guide nucleic acid molecule and an endonuclease, wherein the complex is capable of regulating the expression or activity of the target gene in the cell,
   further optionally wherein:
   (a) the endonuclease is a Cas protein; and/or
(8) the guide nucleic acid molecule does not comprise a ribozyme; and/or
(9) the polynucleotide sequence comprises the structure:

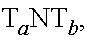

wherein: (i) Tₐ is a first poly T sequence; (ii) T_{b} is a second poly T sequence; (iii) a and b are integers greater than or equal to 4; and (iv) Nis an intervening sequence comprising at least one nucleobase that is not T,
   further optionally wherein a and b are integers greater than or equal to 7; and/or
(10) the polynucleotide sequence comprises the structure:

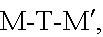

wherein: (i) T is the polyT sequence; (ii) M and M′ are polynucleotide sequences that are at least partially complementary to one another; and (iii) “-” is a polynucleotide linker or absent; and/or
(11) a polynucleotide sequence of M and an additional polynucleotide sequence M′ exhibit at least about 50% sequence identity to the pair of polynucleotide sequences, respectively, selected from the group consisting of (1) SEQ ID NO: 17 and SEQ ID NO: 54; (2) SEQ ID NO: 18 and SEQ ID NO: 55; (3) SEQ ID NO: 19 and SEQ ID NO: 56; (4) SEQ ID NO: 20 and SEQ ID NO: 57; (5) SEQ ID NO: 21 and SEQ ID NO: 58; (6) SEQ ID NO: 22 and SEQ ID NO: 59; (7) SEQ ID NO: 23 and SEQ ID NO: 60; (8) SEQ ID NO: 24 and SEQ ID NO: 61; (9) SEQ ID NO: 26 and SEQ ID NO: 62; (10) SEQ ID NO: 27 and SEQ ID NO: 63; (11) SEQ ID NO: 28 and SEQ ID NO: 64; (12) SEQ ID NO: 29 and SEQ ID NO: 65; (13) SEQ ID NO: 30 and SEQ ID NO: 66; (14) SEQ ID NO: 31 and SEQ ID NO: 67; (15) SEQ ID NO: 32 and SEQ ID NO: 68; (16) SEQ ID NO: 33 and SEQ ID NO: 69; (17) SEQ ID NO: 34 and SEQ ID NO: 70; and (18) SEQ ID NO: 35 and SEQ ID NO: 71, and a complementary sequence pair thereof,
   further optionally wherein:
   (i) the polynucleotide sequence of M and the additional polynucleotide sequence M′ exhibit at least about 60% sequence identity to the pair of polynucleotide sequences, respectively, selected from the group consisting of (1)-(18); and/or
   (ii) the polynucleotide sequence of M and the additional polynucleotide sequence M′ exhibit at least about 80% sequence identity to the pair of polynucleotide sequences, respectively, selected from the group consisting of (1)-(18).

Additional details of heterologous genetic circuits (HGC) and uses thereof are provided in International Application No. PCT/US2018/052211 (entitled “CRISPR/CAS SYSTEM AND METHOD FOR GENOME EDITING AND MODULATING TRANSCRIPTION”), International Application No. PCT/US2023/013240 (entitled “SYSTEMS FOR CELL PROGRAMMING AND METHODS THEREOF), and Clarke et al., Molecular Cell, 81, 226-238, 2021 (entitled “Sequential Activation of Guide RNAs to Enable Successive CRISPR-Cas9 Activities”), each of which is incorporated herein by reference in its entirety.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-43. (canceled)

44. A system for regulating expression or activity of a target gene, the system comprising:
a polynucleotide sequence encoding a guide nucleic acid molecule, wherein the guide nucleic acid molecule exhibits specific affinity to the target gene, to regulate the expression or the activity of the target gene,
wherein the polynucleotide sequence comprises a domain that (i) corresponds to a tetraloop region of the guide nucleic acid molecule, and (ii) comprises a polyT sequence, wherein the polyT sequence does not correspond to a terminal domain of the guide nucleic acid molecule, wherein the polyT sequence is sufficient to reduce expression of the guide nucleic acid molecule, thereby regulating expression or activity of the target gene.

45. The system of claim 44, wherein a size of the polyT sequence is greater than or equal to a threshold length, wherein the threshold length is sufficient to reduce expression of the guide nucleic acid molecule from the polynucleotide sequence.

46. The system of claim 45, wherein the polyT sequence comprises at least 7 T.

47. The system of claim 45, wherein the polyT sequence comprises at least 8 T.

48. The system of claim 45, wherein the polyT sequence comprises at least 9 T.

49. The system of claim 45, wherein the polyT sequence comprises between 6T and 15 T.

50. The system of claim 44, wherein the polyT sequence comprises one or more additional nucleotides that are not T.

51. The system of claim 44, wherein the polyT sequence flanks an intervening sequence that is not a polyT sequence.

52. The system of claim 44, wherein the polynucleotide sequence further comprises an insulator sequence, wherein the insulator sequence is located adjacent to the polyT sequence, and wherein the insulator sequence comprises a sequence which is targetable by a gene editing moiety.

53. The system of claim 52, wherein the insulator sequence is fully complementary.

54. The system of claim 52, wherein the insulator sequence comprises a non-complementary stem region.

55. The system of claim 44, wherein the polynucleotide sequence comprises the structure: 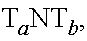
wherein: (i) Tₐ is a first poly T sequence; (ii) T_{b} is a second poly T sequence; (iii) a and b are integers greater than or equal to 4; and (iv) N is an intervening sequence comprising at least one nucleobase that is not T.

56. The system of claim 56, wherein a and b are integers greater than or equal to 7.

57. The system of claim 44, wherein the polynucleotide sequence comprises the structure: 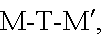
wherein: (i) T is the polyT sequence; (ii) M and M′ are polynucleotide sequences that are at least partially complementary to one another; and (iii) “-” is a polynucleotide linker or absent.

58. The system of claim 57, wherein a polynucleotide sequence of M and an additional polynucleotide sequence M′ exhibit at least about 50% sequence identity to the pair of polynucleotide sequences, respectively, selected from the group consisting of (1) SEQ ID NO: 17 and SEQ ID NO: 54; (2) SEQ ID NO: 18 and SEQ ID NO: 55; (3) SEQ ID NO: 19 and SEQ ID NO: 56; (4) SEQ ID NO: 20 and SEQ ID NO: 57; (5) SEQ ID NO: 21 and SEQ ID NO: 58; (6) SEQ ID NO: 22 and SEQ ID NO: 59; (7) SEQ ID NO: 23 and SEQ ID NO: 60; (8) SEQ ID NO: 24 and SEQ ID NO: 61; (9) SEQ ID NO: 26 and SEQ ID NO: 62; (10) SEQ ID NO: 27 and SEQ ID NO: 63; (11) SEQ ID NO: 28 and SEQ ID NO: 64; (12) SEQ ID NO: 29 and SEQ ID NO: 65; (13) SEQ ID NO: 30 and SEQ ID NO: 66; (14) SEQ ID NO: 31 and SEQ ID NO: 67; (15) SEQ ID NO: 32 and SEQ ID NO: 68; (16) SEQ ID NO: 33 and SEQ ID NO: 69; (17) SEQ ID NO: 34 and SEQ ID NO: 70; and (18) SEQ ID NO: 35 and SEQ ID NO: 71, and a complementary sequence pair thereof.

59. The system of claim 58, wherein the polynucleotide sequence of M and the additional polynucleotide sequence M′ exhibit at least about 60% sequence identity to the pair of polynucleotide sequences, respectively, selected from the group consisting of (1)-(18).

60. The system of claim 59, wherein the polynucleotide sequence of M and the additional polynucleotide sequence M′ exhibit at least about 80% sequence identity to the pair of polynucleotide sequences, respectively, selected from the group consisting of (1)-(18).

61. A method for regulating expression or activity of a target gene in a cell, the system comprising:
contacting the cell with a polynucleotide sequence encoding a guide nucleic acid molecule, wherein the guide nucleic acid molecule exhibits specific affinity to the target gene, to regulate the expression or the activity of the target gene,
wherein the polynucleotide sequence comprises a domain that (i) corresponds to a tetraloop region of the guide nucleic acid molecule, and (ii) comprises a polyT sequence, wherein the polyT sequence is sufficient to reduce expression of the guide nucleic acid molecule, thereby regulating expression or activity of the target gene.
